(19) United States
(12) Patent Application Publication
(10) Pub. No.: US 2023/0147720 A1
(43) Pub. Date: May 11, 2023

(54) Purified Enriched Population Exosomes Derived From Individuals With A Chronic Progressive Lung Disease For Noninvasive Detection, Staging, And Medical Monitoring Of Disease Progression

(71) Applicant: SPIRITUS THERAPEUTICS, INC., New York, NY (US)

(72) Inventors: Sharon Elliot, Miami, FL (US); Marilyn Glassberg Csete, Miami, FL (US)

(73) Assignee: SPIRITUS THERAPEUTICS, INC., New York, NY (US)

(21) Appl. No.: 17/980,271

(22) Filed: Nov. 3, 2022

Related U.S. Application Data
(60) Provisional application No. 63/276,494, filed on Nov. 5, 2021.

(51) Int. Cl. G01N 33/68; C12N 15/113

(52) U.S. Cl. CPC G01N 33/6893; C12N 15/113; C12Q 2600/178; C12N 2310/141; C12Q 2600/158

(57) ABSTRACT

The present disclosure provides a method for noninvasively diagnosing and staging a progressive chronic lung disease characterized by disease related lung dysfunction by deriving from a biological sample from a subject a purified enriched population of exosomes in the biological sample, wherein dysregulated expression of the two or more microRNAs, compared to a healthy control, comprises a signature of a fibrotic lung disease; and medically managing the diagnosed fibrotic lung disease as early as possible in the course of progression of the disease to reduce or slow its progression. The method may identify interstitial pulmonary fibrosis (IPF) at a stage before standard procedures (e.g., Ashcroft scoring and histology) demonstrate changes consistent with lung fibrosis.

### CROSS-REFERENCE TO RELATED APPLICATIONS

This application claims the benefit of priority to U.S. provisional application 63/276,494 (filed 5 Nov. 2021) entitled URINE-DERIVED EXOSOMES FROM INDIVIDUALS WITH IPF CARRY PRO-FIBROTIC CARGO AND IMPAIR TISSUE REPAIR, the contents of which are incorporated herein in their entirety.

### BACKGROUND OF THE INVENTION

Over 200,000 older Americans currently suffer from chronic age-associated fibrotic lung disease annually. Alveolar epithelial cell damage, proliferation of fibroblasts and extracellular matrix (ECM) accumulation lead to irreversible disruption of the lung architecture. While the etiology of IPF remains unknown, there is an ongoing need for diagnostic and prognostic biomarkers of disease.

Extracellular vesicles (EVs), including exosomes and microvesicles, are potential candidates for elucidation of a biomarker signature for IPF and other fibrotic diseases. As a key component of cell-cell communication, EVs deliver miRNAs, mRNA, and tRNA into target cells. The ease of collection and presence of exosomes in most body fluids, including blood, breast milk, saliva, urine, bile, pancreatic juice, cerebrospinal and peritoneal fluids, have facilitated recent studies investigating their use as diagnostic and prognostic markers of disease. Because exosomes are released by all cells and contain cargo from the cell of origin, it is conceivable that they deliver pathogenic materials leading to a disease phenotype. Whether the content of exosomes from different sources are the same has not been studied previously.

Molecular analysis of lung biopsies from individuals with IPF reveal a unique mRNA transcriptome compared with the mRNA transcriptome found from non-fibrotic lung biopsy samples providing evidence supporting the disease conferring properties of exosomes. In serum isolated from individuals with IPF compared to control serum samples, there were 47 differentially expressed miRNAs. Njock et al. reported a signature of miR-142-3p, miR-33a-5p, let-7d-5p in sputum of individuals with IPF. Parimon et al. found that in lung tissue EVs isolated from mouse alveolar spaces and IPF lungs, TGF-β and Wnt signaling were increased after Bleo treatment. These data trigger a hypothesis that dysregulation of miRNAs packaged in exosomes in diseased sources are involved in the development and progression of fibrosis.

### Extracellular Vesicles

Extracellular vesicles (EVs) are nano-sized (typically 50-200 nm in diameter) vesicles of different sizes, cargo, and surface markers that are secreted into the extracellular environment through a variety of mechanisms. They carry various components of the cytoplasm and cell membrane that are selectively loaded into these vesicles. They are secreted by all forms of living cells and play essential roles in different physiological functions and pathological processes. They also have been utilized as diagnostic markers and therapeutic tools in several conditions.

Three types of EVs are biologically distinguishable from one another via the distinct processes through which they are released by the cell. However, the experimental classification of these vesicles is less clear as there is no consensus on what criteria to use for their differentiation. Microvesicles (MVs), or ectosomes (200-2000 nm) arise by direct budding through the cell membrane to the outside of the cell. [Shao, H. et al. Chem Rev. (2018) 118 (4): 1917-50] Microvesicles are enriched in integrins, selectins, and CD40. Exosomes are produced through the inward invagination of the endosomal membrane pathway. [Id., citing Thery, C. et al. Nat. Rev. Immunol. (2002) 2: 569-79; Kowal, J. et al. Curr. Opin. Cell Biol. (2014) 29: 116-25]. The first invagination of the plasma membrane forms a cup-shaped structure that includes cell-surface proteins and soluble proteins associated with the extracellular milieu. This leads to the de novo formation of an early-sorting endosome (ESE) and in some cases may directly merge with a preexisting ESE. The trans-Golgi network and endoplasmic reticulum can also contribute to the formation and the content of the ESE (Kalluri, R., LeBleu, VS. Science (2020) 367 (6478): eaau6977, citing Kalluri, R. J. Clin. Invest. (2016) 126: 1208-15; van Neil, G. et al. Nat. Rev. Mol. Cell Biol. (2018) 19: 213-28; McAndrews, KM, Kalluri, R. Mol. Cancer (2019) 18: 52; Mathieu, M. et al. Nat. Cell Biol. (2019) 21: 9-17; Willms, E. et al. Front. Immunol. (2018) 9: 738; Hessvik, NP, Llorente, A. Cell Mol. Life Sci. (2018) 75: 193-208). Small vesicles can be formed by further inward budding of the limiting membrane inside an endosome, leading to the formation of a multivesicular body (MVB), characterized by the presence of intraluminal vesicles. [Shao, H. et al. Chem Rev. (2018) 118 (4): 1917-50, citing Piper, R C, Katzmann, D J. Annu. Rev. Cell Dev. Biol. (2007) 23: 519-47] During this process, cytosolic contents, transmembrane and peripheral proteins are incorporated into the invaginating membrane [Id., citing Hurley, J H, Hanson, P I. Nat. Rev. Mol. Cell Biol. (2010) 11: 556-66]. ESEs can mature into late-sorting endosomes (LSEs) and eventually generate MVBs, which are also called multivesicular endosomes. MVBs form by inward invagination of the endosomal limiting membrane (that is, double invagination of the plasma membrane). MVBs may then fuse with the lysosome, leading to the degradation of vesicular contents. [Id., citing Vlassov, A V et al. Biochim. Biophys. Acta (2012) 1820: 940-48] Alternatively, MVBs may fuse with the plasma membrane of the cell, releasing vesicles in an exocytotic fashion to the extracellular space. [Id., citing Colombo, M. et al. Annu. Rev. Cell Dev. Biol. (2014) 30: 255-89, Thery, C. F1000 Biol. Rep. (2011) 3: 15] The released exosomes are small membrane-bound lipid vesicles that have a diameter ranging from 30-200 nm. Because of the double invagination processes, protein topology in exosomes is in the same orientation as in the plasma membrane of cells. Apoptotic bodies (APBs) (800-5000 nm diameter) arise from the cell during the process of apoptosis, and emerge either by separation of the membrane blebs or from apoptopodia that arise during the process of apoptosis. [Xu X., et al. Biosci. Rep. (2019) 39 (1): BSR20180992].

Collectively, EVs contain an abundance of cellular cargos [Id., citing Kalra, H. et al. PLoS Biol. (2012) 10: e100450; Keerthikumar, S. et al. J. Mol. Biol. (2016) 428: 688-92; Choi, D S et al. Mass Spectrom. Rev. (2015) 34: 474-90]. Consistent with their biogenesis, the membrane composition of microvesicles reflects most closely the plasma membrane of the parent cells [Id., citing Yanez-Mo, M. et al. J. Extracell. Vesicles (2015) 4: 27066]. Consistent with their endosomal origin, the lipid membrane of exosomes is rich in cholesterol, sphingomyelin, and ceramide that are typical of lipid rafts. [Tan, S S H et al. Tissue Engineering: Part B (2020) doi: 10.1089/ten.teb.2019.0326].

In contrast, a specific subset of endosomal proteins has been identified in exosomes, suggesting a sorting mechanism during exosomal development. The endosomal sorting complex requiredfor transport (ESCRT) has been extensively characterized for regulating and channeling specific molecules into the intraluminal vesicles of the MVBs [Shao, H. et al. Chem Rev. (2018) 118 (4): 1917-50, citing Hurley, J H, Hanson, P I. Nat. Rev. Mol. Cell Biol. (2010) 11: 556-66; Henne, W M et al. Dev. Cell (2011) 21: 77-91]. The ESCRT, with its four main complexes (ESCRT 0, I, II, and III) is responsible for delivering ubiquitinated proteins for lysosomal degradation and protein recycling [Id., citing Wollert, T., Hurley, J H. Nature (2010) 464: 864-9]. Studies have shown that the depletion of specific ESCRT-family proteins can alter the protein content of exosomes and the rate of exosome release from cells [Id., citing Colombo, M. et al., J. Cell Sci. (2013) 126: 5553-65]. Components of the ESCRT system, such as TSG101 and Alix [Id., citing Kowal, J. et al. Curr. Opin. Cell Biol. (2014) 29: 116-25] are found enriched in exosomes and thus are used as markers for exosome identification [Id., citing Lotvall, J. et al. J. Extracell. Vesicles (2014) 3: 26913].

Other ESCRT-independent processes also seem to participate, possibly in an intertwined manner, in exosome formation and release. As such, exosomes are also enriched with molecules involved in ESCRT-independent mechanisms. For example, the tetraspanin proteins such as CD9, CD63 and CD81 have been shown to participate in endosomal vesicle trafficking [Id., citing van Neil, G. et al. Dev. Cell. (2011) 21: 708-21; Verweij, F J et al., EMBO J. (2011) 30: 2115-29] The involvement of the Rab family of small GTPases in vesicle trafficking and fusion with the plasma membrane also suggests a role of these proteins in exosome release [Id., citing Vanlandingham, P A, Ceresa, B P. J. Biol. Chem. (2009) 284: 12110-24; Ostrowski, M. et al. Nat. Cell Biol. (2010) 12: 19-30; Zeigerer, A. et al. Nature (2012) 485: 465-70]. In addition, sphingomyelinase has been demonstrated to be involved in vesicle release, as supported by elevated levels of ceramide in exosomes and a reduction in exosome release upon inhibition of sphigomyelinase [Id., citing Trajkovic, K. et al. Science (2008) 319: 1244-7].

### Proteins Enriched in EVs

EV proteins derive mainly from cellular plasma membrane, cytosol, but not from other intracellular organelles (e.g., Golgi apparatus, endoplasmic reticulum, and nucleus) [Id., citing Simpson, R J et al. Expert Rev. Proteomics (2009) 6: 267-83; Raimondo, F. et al. Proteomics (2011) 11: 709-20; Choi, D S et al. Mass Spectrum Rev. (2015) 34: 474-90]. This protein constitution of EV is indicative of vesicle biogenesis and cargo sorting [Id., citing Kowal, J. et al. Proc. Natl Acad. Sci. USA (2016) 113: E968-77]

### Membrane Proteins

In mammalian vesicles, both transmembrane and lipid-bound extracellular proteins (e.g., lactadherin) are found associated with microvesicles and exosomes [Id., citing Lotvall, J. et al. J. Extracell Vesicles. (2014) 3: 26913] Within the group of transmembrane proteins, exosomes are enriched with tetraspanins (e.g., CD9, CD63, CD81), a superfamily of proteins with four transmembrane domains [Id., citing van Niel, G. et al. Dev. Cell (2011) 21: 708-21; Velrweij, F J et al. EMBOJ. (2011) 30: 2115-29]. Tetraspanins are involved in membrane trafficking and biosynthetic maturation, [Id., citing Perez-Hernandez, D. et al. J. Biol. Chem. (2013) 288: 11649-61; Andreu, Z., Yanez-Mo, M. Front. Immunol (2014) 5: 442] and are highly expressed in exosomes. Tetraspanins, however, are not uniquely expressed in exosomes alone.[Id., citing Lotvall, J. et al. J. Extracell. Vesicles (2014) 3: 26913]. Reflecting their derivation from the plasma membrane of cells, EVs are enriched with specific transmembrane protein receptors (e.g., epidermal growth factor receptors/EGFRs6 [Id., citing Al-Nedawi, K. et al. Proc. Natl Acad. Sci. USA (2009) 106: 3794-9] and adhesion proteins (e.g., epithelial cell adhesion molecule/EpCAM [Id., citing Im, H. et al. Nat. Biotechnol. (2014) 32: 490-5; Tauro, B J et al. Mol. Cell Proteomics (2013) 12: 587-98].

### Intravesicular Proteins

EV-associated intravesicular proteins have diverse functions. They include cytosolic proteins that have membrane- or receptor binding capacity, such as TSG101, ALIX, annexins and Rabs, which are involved in vesicle trafficking. EVs are also enriched with cytoskeletal proteins (e.g., actins, myosins, tubulins), molecular chaperones (e.g., heat-shock proteins/HSPs), metabolic enzymes (e.g., enolases, glyceraldehyde 3-phosphate dehydrogenase/GAPDH) and ribosomal proteins [Id, citing Lotvall, J. et al. J. Extracell. Vesicles (2014) 3: 26913; Choi, D S et al. Mass Spectrom. Rev. (2015) 34: 474-90]. It has been reported that EV protein cargoes can be effectively transported to and received by recipient cells to elicit potent cellular responses in vitro and in vivo [Lai, C P et al. Nat. Communic. (2015) 6: 7029; Mittelbrunn, M., Sanchez-Madrid, F. Nat. Rev. Mol. Cell Biol. (2012) 13: 328-35].

### Nucleic Acids

Both exosomes and microvesicles also contain nucleic acids include miRNAs, mRNAs [Id., citing Valadi, H. et al. Nat. Cell Biol. (2007) 9: 654-9; Skog, J. et al. Nat. Cell Biol. (2008) 10: 1470-6], DNA [Id., citing Balaj, L. et al. Nat. Commun. (2011) 2: 180; Thakur, B K et al. Cell Res. (2014) 24: 766-9] and other non-coding RNAs [Id., citing Wei, Z. et al. Nat. Commun. (2017) 8: 1145] RNA types are summarized in Table 1.

TABLE 1

| RNA | Functions | Coding | Typical Size | |
|---|---|---|---|---|
| mRNA | Protein | Yes | 400-12,000 nt, | |
| | translation | | average ≈2100 nt | |
| microRNA (miRNA) | Post-transcriptional | No | 17-24 | nt |
| | gene silencing | | | |
| Y RNA | Component of Ro60 | No | ≈100 | nt |
| | ribonucleoprotein | | | |
| | particle; initiation | | | |
| | factor for DNA | | | |
| | replication | | | |
| Signal Recognition | Component of SRP | No | ≈280 | nt |
| particle RNA (SRP | ribonucleoprotein | | | |
| RNA) | complex that directs | | | |
| | protein trafficking | | | |
| Transfer RNA | Adapter for | No | 76-90 | nt |
| (tRNA) | matching amino | | | |
| | acid to mRNA | | | |
| Ribosomal RNA | RNA component of | No | 185 (1.9 kb) | |
| (rRNA) | ribosomes | | 28S (5.0 kb) | |
| Small | RNA processing | No | ≈150 | nt |
| nuclear RNA | such as | | | |
| (snRNA) | mRNA splicing | | | |
| Small | Guiding chemical | No | 20-24 | nt |
| nucleolar RNA | modifications of | | | |
| (snoRNA) | other RNAs | | | |
| Long | Many, including in- | No | >100 | nt |
| noncoding | transcription and | | | |
| RNA (lncRNA) | post-transcription | | | |
| | regulation | | | |

mRNA

mRNAs are a large family of coding RNA molecules that specify protein sequence information. Studies have reported that EVs contain a substantial proportion of their parent cells' mRNA pool, many of which are cell type-specific mRNA.[Shao, H. et al. Chem Rev. (2018) 118 (4): 1917-50, citing Wei, Z. et al. Nat. Commun. (2017) 8: 1145; Batagov, A O, Kurochkin, I F. Biol. Direct (2013) 8: 12] These mRNA molecules, often in fragmented form, reside within EVs and are protected from RNase degradation. Furthermore, the fraction of polyadenylated mRNA molecules in EVs suggest that some of them (<2 kb) are capable of encoding polypeptides in support of protein synthesis (i.e., functionality in protein translation). This has been confirmed in multiple studies through different translation assays in recipient cells [Id., citing Valadi, H. et al. Nat. Cell Biol. (2007) 9: 654-9; Skog, J. et al. Nat. Cell Biol. (2008) 10: 1470-6; Lai, C P et al. Nat. Commun. (2015) 6: 7029]

miRNA

miRNAs are a class of small, noncoding RNAs (typically 17-24 nucleotides) which mediate post-transcriptional gene silencing usually by targeting the 3′ untranslated region of mRNAs. By suppressing protein translation, EV miRNAs are powerful regulators for a wide range of biological processes [Id., citing Mittelbrunn, M. et al. Nat. Commun. (2011) 2: 282; Redzic, J S et al. Semin. Cancer Biol. (2014) 28: 14-23]. miRNAs can also exist in multiple stable forms when circulating in bodily fluids. For example, in addition to being packaged into EVs, circulating miRNAs can also be loaded onto high-density lipoprotein [Id., citing Vickers, K C et al. Nat. Cell Biol. (2011) 13: 423-33; Wagner, J. et al. Arterioscler. Thromb. Vasc. Biol. (2013) 33: 1392-400] or bound to AGO2 protein outside the vesicles [Id., citing Arroyo, et al. Proc. Natl Acad. Sci. USA (2011) 108: 5003-8; Turchinovich, A. et al. Methods Mol. Biol. (2013) 1024: 97-107]. The distribution of miRNAs within EVs remains unclear [Id., citing Min, P K & Chan, S Y. Eur. J. Clin. Invest. (2015) 45: 860-74; Turchinovich, A. et al. Methods Mol. Biol. (2013) 1024: 97-107; Chevillet, J R et al. Proc. Natl. Acad. Sci. USA (2014) 111: 14888-93]. As in the case of mRNA, miRNA profiles in EVs reflect their cell of origin but differs somewhat from their parental cells. Some miRNAs have been found preferentially sorted into EVs and remaining functional in recipient cells to regulate protein translation.[Id., citing Villarroya-Beltri, C. et al. nat. Commun. (2013) 4: 2980; Koppers-Lalic, D. et al. Cell Rep. (2014) 8: 1649-58; Santangelo, L. et al. Cell Rep. (2016) 17: 799-808; Teng, Y. et al. Nat. Commu. (2017) 8: 14448]

### Other RNA Types

In addition to mRNA and miRNA, many noncoding RNA types have been identified in EVs through next generation sequencing [Id., citing Huang, X. et al. BMC Genomics (2013) 14: 319; Conley, A. et al. RNA Biol. (2017) 14: 305-16]. These RNAs include transfer RNA (tRNA), ribosomal RNA (rRNA), small nuclear RNA (snRNA), small nucleolar RNA (snoRNA), as well as long noncoding RNA (lncRNA) [Id., citing Wei, Z. et al. Nat. Commun. (2017) 8: 1145; Huang, X. et al. BMC Genomics (2013) 14: 319; Crescitelli, R. et al. J. Extracell. Vesicles (2013) 2: 20677].

Small (about 20-30 nucleotide (nt)) noncoding RNAs regulate eukaryotic genes and genomes (Carthew, R W and Sontheimer, E J. Cell (2009) 136: 642-55). This regulation can occur at multiple levels of genome function, including chromatin structure, chromosome segregation, transcription, RNA processing, RNA stability, and translation (Id.). The effects of small RNAs on gene expression and control are generally inhibitory, and the corresponding regulatory mechanisms are therefore collectively subsumed under the heading of RNA silencing (Id.). The central theme that runs throughout is that the small RNAs serve as specificity factors that direct bound effector proteins to target nucleic acid molecules via base-pairing interactions (Id.). Invariably, the core component of the effector machinery is a member of the Argonaute protein superfamily (Id.).

There are three main categories of small RNAs: short interfering RNAs (siRNAs), microRNAs (miRNAs), and piwi-interacting RNAs (piRNAs) (Id.). siRNAs and miRNAs are the most broadly distributed in both phylogenetic and physiological terms and are characterized by the double-stranded nature of their precursors (Id.). In contrast, piRNAs are primarily found in animals, exert their functions most clearly in the germline, and derive from precursors that are poorly understood, but appear to be single stranded (Id.). Where siRNAs and miRNAs bind to members of the Ago clade of Argonaute proteins, piRNAs bind to members of the Piwi clade (Id.).

The signature components of RNA silencing are Dicers, Agos, and ˜ 21-23 nt duplex-derived RNAs (Id.). Both siRNA and miRNA small RNAs depend on Dicer enzymes to excise them from their precursors, and Ago proteins to support their silencing effector functions (Id.).

RNase III enzymes, which are dsRNA-specific nucleases, are the source of miRNA/siRNA biogenesis (Id.). One class of large RNase III enzymes has several domains in a specific order from the amino to carboxy terminus: a DEXD/H ATPase domain, a DUF283 domain, a PAZ domain, two tandem RNase III domains, and a dsRNA-binding domain (Id.). Some members of this family differ slightly from this arrangement (Id.).

The PAZ and RNase III domains play central roles in excising siRNAs preferentially from ends of dsRNA molecules. PAZ domains are shared with Argonaute proteins and are specialized to bind RNA ends, especially duplex ends with short (˜2 nt) 3′ overhangs. An end engages the Dicer PAZ domain, and the substrate dsRNA then extends approximately two helical turns along the surface of the protein before it reaches a single processing center that resides in a cleft of an intramolecular dimer involving the RNase III domains. Each of the two RNase 1111 active sites cleaves one of the two strands, leading to staggered duplex scission to generate new ends with ˜2-3′ nt overhangs. The reaction leaves a 5′ monophosphate on the product ends, consistent with a requirement for this group during later stages of silencing. This general model pertains equally to pre-miRNA stem-loop substrates and to long, perfectly base-paired dsRNAs. In some species, different functional categories of small RNAs exhibit slightly different lengths; this appears to be dictated by the distance between the PAZ domain and the processing center in the relevant Dicer enzyme (Id.).

The roles of the ATPase domain probably vary among different forms of Dicer (Id.). ATP promotes dsRNA processing by Drosophila Dicer 2 and C. elegans Dcr-1, and mutations predicted to cripple ATPase activity in Drosophila Dicer-2 specifically abolish dsRNA processing. In contrast, ATP is dispensable for dsRNA processing by human Dcr (hDcr), and an ATPase defective mutant exhibits no processing defect (Id.).

Dicers isolated from their natural sources generally are found in a heterodimeric complex with a protein that contains two or three double stranded Ras binding domains (dsRBDs); the Ras-binding domain (RBD) is an independent domain of about 75 residues, which is sufficient for GTP-dependent binding of Ras and other G alpha GTPases. Both hDcr and Drosophila Dcr-2 process dsRNAs effectively in the absence of the heterodimeric partner (TRBP and R2D2, respectively). In at least some cases, the role of Dicer in silencing extends beyond dsRNA processing and into the pathway of RISC assembly; this activity is much more dependent on the dsRBD partner protein (Id.).

### Argonautes

The Argonaute superfamily can be divided into three separate subgroups: the Piwi clade that binds piRNAs, the Ago clade that associates with miRNAs and siRNAs, and a third clade described in nematodes. All gene regulatory phenomena involving ˜20-30 nt RNAs are thought to require one or more Argonaute proteins, which are the central, defining components of an RNA-induced silencing complex (RISC). The double-stranded products of Dicer enter into a RISC assembly pathway that involves duplex unwinding, culminating in the stable association of only one of the two strands with the Ago effector protein. This guide strand directs target recognition by Watson-Crick base pairing; the other strand of the original small RNA duplex (the passenger strand) is discarded (Id.).

Argonaute proteins are defined by the presence of four domains: the PAZ domain (shared with Dicer enzymes), the PIWI domain that is unique to the Argonaute superfamily, and the N and Mid domains. The overall protein structure is bi-lobed, with one lobe consisting of the PAZ domain and the other lobe consisting of the PIWI domain flanked by N-terminal (N) and middle (Mid) domains. The Argonaute PAZ domain has RNA 3′ terminus binding activity, and the co-crystal structures reveal that this function is used in guide strand binding. The other end of the guide strand engages a 5′ phosphate binding pocket in the Mid domain, and the remainder of the guide tracks along a positively charged surface to which each of the domains contributes. The protein-DNA contacts are dominated by sugar-phosphate backbone interactions. Guide strand nucleotides 2-6, which are especially important for target recognition, are stacked with their Watson-Crick faces exposed and available for base pairing (Id.).

The PIWI domain adopts an RNase H-like fold that in some cases can catalyze guide strand-dependent endonucleolytic cleavage of a base pair target. This initial cut represents the critical first step in a subset of small RNA silencing events that proceed through RNA destabilization. Not all Argonaute proteins have endonucleolytic activity, and those that lack it usually also lack critical active-site residues that coordinate a presumptive catalytic metal ion (Id.).

In humans, four of the eight Argonaute proteins are from the Ago clade and associate with both siRNAs and miRNAs (Id.).

### MicroRNA Biogenesis

MicroRNAs are found in plant and animal branches of Eukaryotes and are encoded by a bewildering array of genes. Transcription of miRNAs is typically performed by RNA polymerase II, and transcripts are capped and polyadenylated. Although some animal miRNAs are individually produced from separate transcription units, many more are produced from transcription units that make more than one product. A transcript may encode clusters of distinct miRNAs, or it may encode miRNA and protein. The latter type of transcript is organized such that the miRNA sequence is located within an intron. Many new animal miRNAs are thought to arise from accumulation of nucleotide sequence changes and not from gene duplication (Carthew, R W and Sontheimer, E J. Cell (2009) 136: 642-55).

The resulting primary or pri-miRNA transcript extends both 5′ and 3′ from the miRNA sequence, and two sequential processing reactions trim the transcript into the mature miRNA. Processing depends on the miRNA sequence folding into a step-loop structure. A typical animal pri-miRNA consists of an imperfectly paired stem of ˜33 bp, with a terminal loop and flanking segments. The first processing step, which occurs in the nucleus, excises the stem-loop from the remainder of the transcript to create a pre-miRNA product. For most pri-miRNAs, a nuclear member of the RNase III family (Drosha in animals) carries out this cleavage reaction. Although Drosha catalyzes pri-miRNA processing, it depends on a protein cofactor, which contains two dsRBD domains and stably associates with the ribonuclease to form the microprocessor complex (Id.).

An alternative pathway uses splicing of pri-miRNA transcripts to liberate introns that precisely mimic the structural features of pre-miRNAs. These introns then enter the miRNA processing pathway without the aid of the Microprocessor (Id.).

The second processing step excises the terminal loop from the pre-miRNA stem to create a mature miRNA duplex of approximately 22 bp length. In animals, the pre-miRNA is exported from the nucleus, and the canonical Dicer enzyme carries out the cleavage reaction in the cytoplasm (Id.).

MicroRNAs behave like traditional polymeric products of gene activity, such that most species of a miRNA have highly exact ends, although there is a little variation. This feature of miRNAs may allow them to interact with greater specificity on substrate mRNAs without a need for stringent complementarity or large overlap (Id.).

Consequently, the processing machinery is constructed to produce miRNA duplexes with highly exact ends. The first cut, carried out by Drosha with the aid of its dsRBD domain binding partner protein (called DGCR8), is most critical. DGCR8 directly interacts with the pri-miRNA stem and flanking single-stranded segments. The cleavage site is determined by the distance from the stem-flank junction, which is precisely one turn of a dsRNA helix (11 bp) and is the minimal processing length for an RNase III enzyme. Although Drosha carries out the cleavage reaction, it relies upon DGCR8 to serve as a molecular anchor that properly positions Drosha's catalytic site the correct distance from the stem-flank junction. Thus, the endpoint of the stem is a critical determinant for one end of the mature miRNA (Id.).

The second cut performed by Dicer defines the other end of the mature miRNA. Dicer will cleave anywhere along a dsRNA molecule but has a strong preference for the terminus. The PAZ domain of Dicer interacts with the 3′ overhang at the terminus and determines the cleavage site in a ruler-like fashion. The RNase III catalytic sites are positioned two helical turns or 22 bp away from the terminus/PAZ portion of the Dicer-RNA complex (Id.).

While regulation of miRNA biogenesis has not been extensively studied, a surprising number of miRNA genes are formed under the control of the very targets that they regulate. A rationale behind these double-negative regulatory relationships is that tight regulation of miRNA biogenesis is crucial. Mis-expression of miRNAs frequently mimics loss of function phenotypes for their targets. This would be prevented if biogenesis of a miRNA is strictly controlled by its targets. The restriction would also explain how off-targeting effects by wayward miRNAs are carefully limited (Id.).

### MicroRNA Associations

The mature miRNA duplex is a short-lived entity; it is rapidly unwound when it associates with an Ago protein. Unwinding occurs so rapidly after duplex formation, because the two processes are physically coupled due to Ago2's presence in a complex with Dicer and TRBP, the double-stranded RNA binding protein that loads siRNA into the RISC (Id.).

miRNA unwinding is accompanied by differential strand retention, i.e., one strand is retained while the other strand is lost. Strand retention is based on the relative thermodynamic stability of the duplex's ends. Although the rule is that the 5′ terminus of the retained strand is at the less stably base-paired end of the duplex, this rule is not absolute. The other strand is appreciably detected in Ago complexes, lending ambiguity to the notion of strand asymmetry. Although either strand can become stably associated with Ago proteins, the more commonly associate strand is termed the miRNA strand; the other strand is called the miRNA* strand. miRNA unwinding is not accompanied by cleavage of the ejected strand by the associated Ago (Id.).

The mammalian Dicer/Ag/miRNA complex is associated with other proteins, e.g., Gemin3, Gemin4, Mov10, and Imp8, as well as the mammalian protein GW182, associate with Ago2. GW182 is both necessary and sufficient for miRNA-bound Ago to silence gene expression. Thus miRNA-bound Ago in association with GW182 can be thought of as the miRISC complex (Id.).

Post-Transcriptional Repression by miRNAs

An miRNA acts as an adaptor for miRISC to specifically recognize and regulate particular mRNAs. If miRISC is tethered to a heterologous RNA recognition factor, the factor enables miRISC to recognize and repress mRNAs that lack miRNA-binding sites. With few exceptions, miRNA-binding sites in animal mRNAs lie in the 3′ untranslated region (UTR) and are usually present in multiple copies. Most animal miRNAs bind with mismatches and bulges, although a key feature of recognition involves Watson-Crick base pairing of miRNA nucleotides 2-8, representing the seed region (Id.).

While it was thought that perfect complementarity allows Ago-catalyzed cleavage of the mRNA strand, whereas central mismatches exclude cleavage and promote repression of mRNA translation, it appears that translational repression is the default mechanism by which miRNAs repress gene expression, both in animals and plants. Perfectly complementary miRNAs may additionally engage in mRNA cleavage such that their effects are the result of both mechanisms (Id.).

The mechanisms by which miRISC regulates translation have been subject to ongoing debate. The fundamental issue of whether repression occurs at translation initiation or post-initiation has not yet been resolved. There are three competing models for how miRISC represses initiation. One proposes that there is competition between miRISC and elF4E for binding to the mRNA 5′ cap structure. A second model has proposed that miRISC stimulates de-adenylation of the mRNA tail; translation is repressed because the cap and PABP1-free tail of the deadenylated mRNA are unable to circularize. A third model has proposed that miRISC blocks association of the 60S ribosomal subunit with the 40S preinitiation complex, i.e., the recruitment of eIF6 by miRISC may repress translation by preventing the assembly of translationally competent ribosomes at the start codon (Id.).

It is unclear why some targets are degraded and others are not (Id.).

Without being limited by any particular theory, it appears that the mode of regulation of any miRNA (repression vs. activation) in the context of the whole cell and the myriad activities that affect posttranscriptional gene regulation may be context dependent (Id.).

The cell's position in the cell cycle is one such context. For example, miRNA let-7 and an artificial miRNA (CXCR-4) repress translation in proliferating human cells, but change into translational activators when the cell cycle is arrested at the G1 checkpoint by serum starvation. Aphidicollin-induced arrest at G1 also generates translational activation, whereas nocodazole-induced arrest at G2/M generates translational repression. Lymphocyte growth arrest induces TNFα expression that is required for macrophage maturation; miR-369-3p switches from a repressor to an activator of TNFα translation when cells in culture are growth arrested (Id., citing Vasudevan, S. et al. Science (2007) 318: 1931-34).

Binding site position is another context. Interaction of miR-10a with the 5′UTR of certain ribosomal subunit mRNAs leads to their activated translation, whereas interaction with the 3′UTR leads to repression (Id., citing Orom, U A et al. (2008) Mol. Cell 30: 460-71).

Another context is how small RNA regulation is organized and modulated within the cell. Ago proteins are frequently associated with membrane trafficking compartments, such as the Golgi and ER (Id., citing Cikaluk, D. E. et al. Mol. Biol. Cell (1999) 10: 3357-72). It has been hypothesized that miRISC factors might become anchored in certain subcellular compartments, e.g., P bodies or GW bodies, two separate pools of sequestered non-translating RNAs (Patel, P H, et al. PLos One (2016) 11(3): e015029). Subunits of miRISC (miRNAs, Ago and GW1821) and their repressed targets also are enriched in GW bodies. While GW bodies are not essential for miRNA repression, GW body formation requires an intact miRNA pathway (Carthew, R W and Sontheimer, E J. Cell (2009) 136: 642-55).

miRNA Expression

MicroRNAs regulate gene expression at the post-transcriptional level. The exact functional outcome of an miRNA may be determined by multiple features, including the cell type affected, the inducing signal, and the transcriptomic profile of the cell, which ultimately affect the availability and ability to engage different target mRNAs and bring about its unique responses. Indeed, data suggest that miRNAs may play different roles in diverse biological contexts. [Lee, H-M et al. BMB Rep. (2016) 49 (6): 311-18].

miR-29: levels of miR-29, including miR-29a, miR-29b, and miR-29c, are significantly lower in fibrotic livers as shown in human liver cirrhosis, as well as in two different fibrotic animal molecules (carbon tetrachloride and bile duct ligation, while their down regulation affects hepatic stellate cell (HSC) activation [Huang, Y-H et al. Intl J. Molec. Sci. (2018) 19: 1889, citing Mann, J. et al. Gastroenterology (2010) 138: 705-14; Roderburg, C. et al. Hepatology (2011) 53: 209-18; Sekiya, Y. et al. Biochem. Biophys. Res. Commun. (2011) 412: 74-9]. It has been reported that TGF-β1 was capable of mediating the downregulation of miR-29 in HSCs [Id., citing Roderburg, C. et al. Hepatology (2011) 53: 209-18]; the same was reported in the study of Bandyopadhyay et al. who found this effect to be specific to HSC [Id., citing Bandyopadhyay, S. et al. J. Infect. Dis. (2011) 203: 1753-62]. The overexpression of miR-29 in murine HSC results in the down regulation of collagen expression, including collagen-lca and collagen-4α1 [Id., citing Roderburg, C. et al. Hepatology (2011) 53: 209-18; Bandyopadhyay, S. et al. J. Infect. Dis. (2011) 203: 1753-62; Huang, J. et al. Int. J. Mol. Sci. (2014) 15: 9360-71] by directly targeting the mRNA expression of these extracellular matrix genes.

miR-29 family clusters also have emerged as a major anti-fibrotic player in kidney fibrosis associated with Smad-dependent and Smad-independent pathways (Srivastava, S P et al. Front. Pharmacol. (2019) 10: 904, citing Chung A C and Lan H Y. Front. Physiol. (2015) 6: 50). The expression level of members of the miR-29 family is significantly suppressed in both renal fibrosis (Id., citing Lan, H Y. Clin. Exp. Pharmacol. Physiol. (2012) 39: 731-38; Meng, X M et al. Clin Sci. (London) (2013) 124: 243-54; Srivastava, S P et al Fibrogenesis Tissue Repair (2014) 7: 12) and diabetic (Id., citing Srivastava, S P et al. Sci Rep. (2016) 6: 29884) and hypertensive nephropathy (Id., citing Wei, Q. et al. IUBMB Life (2013) 65: 602-14). miR-29 is downstream of Smad3 and can suppress upstream TGFβ-Smad3 signaling by miR-29b-mediated negative feedback (Id., citing He, Y. et al. Biochimie (2013) 95: 1355-59). miR-29b binds to the coding region of TGFβ1 mRNA at exon 3, which blocks the translation of TGFβ1, resulting in the suppression of Smad3-dependent fibrosis ((Id., citing Zhang, Y. et al. Mol. Ther. (2014) 22: 974-85). miR-29 binds to the promoter region of smad3 and exerts anti-fibrotic properties. In vitro, overexpression of miR-29 inhibited, but knockdown of miR-29 enhanced, TGFβ1-induced expression of collagens I and III in cultured proximal tubular epithelial cells (TECs) (Id., citing Qin, W. et al. J. Am. Soc. Nephrol. (2011) 22: 1462-74; Wang, B. et al. J. Am. Soc. Nephrol. (2012) 23: 252-65; Qi, R and Yang, C. Cell Death Dis. (2018) 9: 1126). However, ultrasound-mediated gene delivery of miR-29 blocked progressive renal fibrosis in obstructive nephropathy (UUO) (Id., citing Qin, W. et al. J. Am. Soc. Nephrol. (2011) 22: 1462-74; Qi, R and Yang, C. Cell Death Dis. (2018) 9: 1126)). Data from various studies have shown that members of the miR-29 family target different isoforms of collagen and have an anti-fibrotic role (Id., citing Wang, B. et al. J. Am. Soc. Nephrol. (2012)23: 252-65; Qi, R and Yang, C. Cell Death Dis. (2018) 9: 1126). TGFβ1 inhibits the beneficial role of miR-29 family by down-regulating the expression in TECs (Id., citing Du, B. et al. FEBS Lett. (2010) 584: 811-16; Wang, B. et al. J. Am. Soc. Nephrol. (2012) 23: 252-65), mesangial cells (Id., citing Wang, B. et al. J. Am. Soc. Nephrol. (2012) 23: 252-65), and podocytes (Id., citing Wang, B. et al. J. Am. Soc. Nephrol. (2012) 23: 252-65). miR-29b suppression contributes to progressive renal injury in several mouse models of chronic kidney disease (CKD) (Id., citing Qin, W. et al. J. Am. Soc. Nephrol. (2011) 22: 1462-74; Wang, B. et al. J. Am. Soc. Nephrol. (2012) 23: 252-65; Ramdas, V. et al. Am. J. Pathol. (2013) 183: 1885-96); however, overexpression of miR-29b provides a therapeutic benefit in unilateral ureter obstruction (UUO) and db/db obese mice (Id., citing Qin, W. et al. J. Am. Soc. Nephrol. (2011) 22: 1462-74; Chen, H Y et al. Mol. Ther. (2014) 22: 842-53). In db/db mice, miR-29a has been shown to be elevated in the liver and to regulate gluconeogenesis Id., citing Pandey, A K et al. Mol. Cell Endocrinol. (2011) 332: 125-33). Treatment of rats with losartan caused a remarkable increase in the level of miR-29b expression, which was linked with lower expression of collagen, fibronectin, and laminin, and provided protection from kidney fibrosis (Id., citing Wang, B. et al. J. Am. Soc. Nephrol. (2012) 23: 252-65). miR-29 family clusters also inhibit elevated dipeptidyl dipeptidase-4 (DPP-4) protein levels by targeting the 3′UTR of its mRNA (Id., citing Kanasaki, K. et al. Diabetes (2014) 63: 2120-31; Shi, S. et al. Kidney Int. (2015) 88: 479-89). TGF02-mediated induction of DPP-4 and down-regulation of miR-29 are associated with endothelial to mesenchymal transition (EndMT) (Id., citing Kanasaki, K. et al. Diabetes (2014) 63: 2120-31; Shi, S. et al. Kidney Int. (2015) 88: 479-89). miR-29 and TGFβ signaling exhibit a negative feedback loop and regulate each other, as induction of TGFβ signaling suppresses downstream miR-29 (Id., citing Kanasaki, K. et al. Diabetes (2014) 63: 2120-31) and miR-29 suppresses upstream TGFβ signaling (Id., citing Zhang, Y. et al. Mol. Ther. (2014) 22: 974-85), This relationship supports an anti-fibrotic role of miR-29 in kidney fibrosis.

miR 10a: Differential expression of miR-10a has been implicated in regulating a pro-inflammatory endothelial phenotype. Kumar, S. et al. Vascul. Pharmacol. (2019) 114: 76-92, citing Fang, Y. et al. Proc. Natl Acad. Sci. USA (2010) 107 (30) 13450-55]. Stable blood flow upregulates the expression of miR-10a in the endothelium. Loss of miR-10a results in activation of NfxB via MAP3K7 and PTRC, both of which promote IKB degradation and p65 translocation, resulting in endothelial inflammation in a porcine model of atherosclerosis.

In a bleomycin-induced pulmonary fibrosis model in mouse, miRNAs deregulated in the late period (days 14 and 21) after bleomycin injury were demonstrated to target key components in the TGF-β signaling pathway. These miRNAs include miR-196b, miR-704, miR-717, miR-16, miR-195, miR-10a, miR-211, miR-34a, miR-367, miR-21, and let-7f, which target TGF-β family members such as TGF-β2 and 3, TGF-β receptors such as TGF-β receptor I and II, Smad family members including Smad 3, 6, and 7, and procollagen type 1 alpha 2. [Xie, T. et al. Physiol. Genomics (2011) 43 (9): 479-87].

Another study reported that in mouse, the hepatic fibrosis tissue transforming growth factor (TGF)-β1/Smads signal transduction pathway correlated with the progression of hepatic fibrosis. It is known that transforming growth factor (TGF)-β1 induces hepatic fibrosis [Zhou, G. et al. Exp. Ther. Med. (2016) 12 (30: 1719-22), citing Tomita, K. et al. Gut (2006) 55: 415-24], and that Smad protein, a key active substrate of TGF-β1 family receptor kinase [Id., citing Heldin, C H et al. Nature (1997) 390: 465-71] constitutes the negative feedback loop in TGF-β signal transduction and exerts an anti-fibrosis effect [Id., citing Kavsak, P. et al. Mol Cell 1(2000) 6: 1365-75; Padda, R S et al. Am. J. Physiol. Gastroint. Liver Physiol. (2015) 308: G251-61]. Therefore, together, Smad and TGF-β1 cause HSC activation, and initiate collagen gene expression, resulting in the genesis of hepatic fibrosis. Forty healthy female 8-week-old C57BL6/J mice were randomly divided into a control group (intraperitoneal injection of 5 μl/g normal saline, twice per week for 8 weeks) and a hepatic fibrosis group (intraperitoneal injection of 5 μl/g 10% CCI4 olive oil, twice per week for 8 weeks), with 20 mice per group. RT-PCR was used to test miR-10a expression in cells in both groups. Cell culture and transfection of miR-10a mimics were conducted in the two groups and a Cell Counting Kit-8 was used to test the expression of TGF-β1 and Smad7 in hepatic fibroblasts. It was found that miR-Oa expression was significantly increased in the hepatic fibrosis group compared with the control group (P<0.05), and that the expression level of miR-10a was significantly increased in the group transfected with miR-10a mimics compared with the control group (P<0.05). A high expression of miR-10a significantly increased TGF-β1 expression and reduced Smad7 expression in the hepatic fibrosis group (P<0.05) thus exerting a hepatic fibrosis-promoting effect by regulating the TGF-β1/Smads signal transduction pathway. [Zhou, G. et al. Exp. Ther. Med. (2016) 12 (30): 1719-22].

Another study indicated that down-regulation of miR-10a may inhibit collagen formation, reduce atrial structure remodeling, and decrease proliferation of cardiac fibroblasts, eventually suppressing cardiac fibrosis in a rat model of atrial fibrillation via inhibition of the TGF-(β1/Smads signaling pathway. Overexpressed miR-10a significantly prolonged the duration of AF, further elevated the collagen volume fraction (CVF), and increased the viability of CFs in AF rats; these findings were in contrast with the findings for rats with inhibition of miR-10a (all P<0.05). Moreover, miR-10a overexpression could promote miR-10a, collagen-I, collagen III, α-SMA, and TGF-β1 protein expression and increase the levels of hydroxyproline but reduced Smad7 protein expression in atrial tissues and CFs in AF rats. Not surprisingly, inhibiting miR-10a led to completely contrasting results (all P<0.05). Moreover, TGF-β1 treatment could reverse the inhibitory effect of miR-10a down-regulation on cardiac fibrosis in CFs. Bioinformatics analysis and luciferase reporter assay results demonstrated that miR-10a bound directly to the 3′-UTR of BCL6, which is involved in cell growth and proliferation. [Li, P F et al. Biosci. Rep. (2019) 39 (20: BSR20181931).

miR34a: MiR34a inhibits sirtuin-1 and shows increased expression in peripheral lungs and epithelial cells of COPD patients, and is correlated with increased expression of senescence markers in lung cells. [Barnes P J et al. Am. J. Respir. Crit. Care Med. (2019) 200 (5): 556-64, citing Baker, J. et al. Sci. Rep. (2016) 6: 358710]. It also regulates sitruin-6, but not other sirtuins. MiR-34a is increased by oxidative stress through activation of PI3K-mTOR signaling and leading to a parallel reduction in Sirt1 and Sirt6, whereas other sirtuins are unchanged, as in COPD lungs. An antagomir of miR-34 restores sirt1 and sirt6 in senescent small airway epithelial cells from COPD patients, reduces markers of cellular senescence (p16, p21, p53), reduces the SASP response (TNFα, IL-1β, IL-6, CCL2, CXCL8, MMP9), and increases proliferation of senescent epithelial cells by reversing cell cycle arrest. [Id., citing Baker, J. et al. Sci. Rep. (2016) 6: 358710]. miR-34a also is increased in COPD macrophages and may be associated with impaired phagocytosis and uptake of apoptotic cells (efferocytosis) observed in this disease. [Id., citing McCubbrey, Al et al. Immunol. (2016) 196: 1366-75].

One study provided evidence that several miRNAs (e.g., miR-21, miR-34a, let-7e, miR-99b, miR-125a, and miR-342 were coordinately upregulated during monocyte-derived dendritic cell (MDDC) differentiation, and validated two genes (WNT1 and JAG1) targeted by 3 of these miRNAs (miR-21, miR-34a, and let-7e) as being involved in MDDC differentiation. Antagonizing the differential expression of miR-21 and miR-34a by either transfection of miRNA inhibitors or by exogenous addition of Wnt-1 and Jagged-1 resulted in stalling MDDC differentiation, suggesting that this regulatory pathway is necessary for MDCC differentiation.[Hashimi, S T et al. Blood (2009) 114 (2): 404-14].

miR-34a (as well as miR-27a, miR-28a are highly expressed in the myocardium during congestive heart failure. [Climent, M. et al. Intl J. Mol. Sci. (2020) 21: 4370, citing Tian, C. et al. Am. J. Physiol. Hear. Cir. Physiol. (2018) 314: H928-H939].

In the lung, miR-34a has been correlated to the antioxidant function of flaxseed in radiotherapy, and more generally, the miR-34 family members have been suggested to be involved in COPD. [Id, citing Mizuno, S. et al. Chest (2012) 142: 663-73; Christofidou-Solomidou, M. et al. Cancer Biol. Ther. (2014) 15: 930-37; Zhang, L. et al. J. Exp. Clin. Cancer Res. (2019) 38: 53]. In the heart, an increase of miRNA has been observed in pre-diabetic and diabetic patients [Id., citing Kong, L. et al. Acta Diabetol. (2011) 48: 61-69], where high glucose levels are known to induce an accumulation of ROS. MiR-34a was found upregulated in diabetic mouse hearts and to regulate redox signaling pathways [Id. Citing Costantino, S. et al. Eur. Heart J. (2016) 37: 572-6]. Moreover it was reported that miR-34a upregulation in diabetic mice led to dysregulation of endothelial cells by targeting Sirt1 [Id., citing Li, Q. et al. Arterioscler. Thromb. Vasc. Biol. (20016) 36: 2394-2403]. In an independent study, in vitro experiments using cardiomyocytes in high glucose conditions confirmed the induction of high levels of miR-34a and targeting of Sirt1 [Id., citing Zhu, Y. et al. Artif. Cells Nanomed. Biotechnol. (2019) 47: 4172-81], ultimately leading to oxidative stress.

miR125: miR-125a-3p can inhibits antimicrobial responses and host defenses against mycobacterial infection by targeting the gene encoding autophagy UV radiation-resistance-associated protein [Lee, H-M et al. BMB Rep. (2016) 49 (6): 311-18, citing Kim, J K et al. J. Immunol. (2015) 194: 5355-65]. In addition, miR-125a-5p can promote IL-4-induced expression of the alternative M2 phenotype by targeting KLF13, a transcriptional factor that is active during T lymphocyte activation and inflammation [Id., citing Banerjee, S. et al. J. Biol. Chem. (2013) 288: 35428-36]. miR-125a-5p plays an important role in inhibiting the classical M1-type activation induced by LPS stimulation, and also can suppress the phagocytic and bactericidal activities associated with macrophage M1 functionality [Id., citing Banerjee, S. et al. J. Biol. Chem. (2013) 288: 35428-36]. Together, these data suggest that miR-125a may inhibit innate macrophage responses by regulating macrophage differentiation, inflammation, and autophagy. The expression of miR-125b-5p (which has the same core sequence as miR-125a-5p) is modulated by NF-κB signaling. miR-125b-5p targets the 3′UTR region of TNF-α gene to negatively regulate the inflammatory response Id., citing Tili, E. et al. J. Immunol. (2007) 179: 5082-89].

miR-142: The conserved mir142 gene encodes two highly expressed mature miRNAs, 142-3p and 142-5p, that are processed from the same precursor yet have different mRNA targets. [Berrien-Elliott, M. M. et al. Immunity (2019) 479-90] A role for miR-142-3p and/or miR-142-5p in regulating hematopoietic cells has been reported using global Mir142/mouse models and is variable depending on the cells examined. Megakaryocytes, mast cells, dendritic cells, and erythrocytes require miR-142 for their development and function; previous reports have demonstrated a reduction in peripheral T cells and altered B cell development in miR-142-deficient mice [Id., citing Chapnik, E. et al. J. Exp. Med. (2018) 2717-31, Kramer, N J et al. Blood (2015) 125: 3720-30; Mildner, A. et al. Blood (2013) 121: 1016-27; Rivkin, N. et al. 2017; Yamada et al Haematologica (2017) 102: e476-80]. miR-142-3p and/or miR-142-5p have also been shown to be important for mature T cell effector responses and proliferation [Id., citing Su et al. Nat. Commun. (2015) 6: 8523]. There is evidence that miR-142 is critically required for IL-15 receptor signaling and NK cell survival, whereas loss of miRNA-142 results in distinct TGF-β- and/or integrin-supported type 1 innate lymphoid cells (ILCs). Natural killer (NK) cells are cytotoxic type 1 ILCs that defend against viruses and mediate anti-tumor responses. To protect the host, NK cells both release cytokines (e.g., interferon-γ) and kill target cells [Id., citing Lanier, L L. Nat. Rev. Immuno. (20008) 8: 259-68]. Like NK cells, ILC1s produce IFN-γ in response to cytokine receptor signals but localize to different sites in vivo and are poorly cytotoxic. NK cells are found throughout lymphoid organs and tissues, whereas ILC-1 cells are found primarily in non-lymphoid tissues [Id., citing Diefenbach, A. et al. Immunity (2014) 41: 354-65].

miR 181: The evolutionary conserved miR-181 family consists of 6 members, genetically clustered into pairs, miR-181a/b-1 (on chromosome 1 in mice and humans), miR-181a/b-2 (on chromosome 2 in mice and 9 in humans), and miR-181c/d (on chromosome 8 in mice and 19 in humans). [Grewers, Z. and Krueger, A. Intl J. Mol. Sci. (2020) 21 (17): 6200]. miR-181a is dynamically expressed in T cells over the lifetime of an organism, with its levels progressively decreasing with increasing age [Id., citing Palin, A C et al. J. Immunol. (2013) 190: 2682-91; Li, g. et al. Nat. Med. (2012) 18: 1518-24]. An ectopic increase of miR-181a expression in mature T cells resulted in increased sensitivity to peptide antigens as well as increased basal phosphorylation levels of Lck and Erk. Likewise, the inhibition of miR-181a via antagomirs reduced TCR sensitivity. Consistent with its role in modulation of TCR signaling, multiple phosphatases involved in T cell receptor signaling such as SHP-2, PTPN22, DUSP5, or DUSP6, were identified as targets of miR-181a [Id., citing Li, Q.-J et al. Cell (2007) 129: 147-61]. Although SHP-1 was not directly repressed, ectopic expression of miR-181a interfered with SHP-1 forming a physical interaction with Lck, suggesting that miR-181a might also indirectly modulate TCR signaling via SHP-1. Accumulating evidence indicates that miR-181a/b-1 predominantly acts as a rheostat of TCR signaling in both thymocytes and peripheral T cells, most likely by interfering with TCR signal strength via a co-targeting network of negative regulatory phosphatases. Consistent with its dynamic expression profile, the consequences of loss of miR-181a/b-1 are generally more severe during intrathymic T cell development, in particular of agonist-selected T cell populations.

MiR-181c has been found to be deleterious in the cardiac setting, but protective in the pulmonary system. Das et al. delivered miR-181c into rats by using nanoparticles and found that it targeted the mitochondrial cytochrome c oxidase subunit 1 (mt-COX1). COX is the last enzyme of the mitochondrial respiratory chain and the major oxygen consumer enzyme in the cells [Climent, M. et al. Intl J. Mol. Sci. (2020) 21: 4370, citing Bourens, M. et al. Antioxid. Redox Signal. (2013) 19: 19940-52; Dennerlein, D. and Rehling, P. J. Cell Sci. (2015) 128: 833-37]. Indeed, by delivering miR-181c, the authors observed a significantly aberrant consumption of oxygen, ROS production, and mitochondrial membrane potential in cardiac mitochondria isolated from miR-181c-nanoparticle-treated animals, suggesting that miR-181c targets mitochondrial genes, therefore causing cardiac dysfunction [Id., citing Das, S. et al. PLoS One (2014) 9: e96820].

In the lung, miR-181c expression levels were found to be low in the tissue of COPD patients and overexpression of this miRNA was shown to inhibit cigarette smoke-induced COPD in mice. MiR-181c was found to target CNN1 (Cysr61) and its overexpression to decrease the inflammatory response, neutrophil infiltration, and inflammatory cytokines induced by cigarette smoking, as well as the reactive oxygen species (ROS) generation [Id., citing Du, Y. et al. Respir. Res. (2017) 18: 155]. However, the exact mechanism by which miR-181c regulates ROS in COPD has not yet been elucidated. Another member of the miR-181 family was also found to cause a reduction of the levels of ROS in the pulmonary system. Jiang et al. discovered that the expression of miR-181a was downregulated in lungs of LPS-challenged mice and that the Toll-Like Receptor 4 (TLR4) was a target of miR-181a. When miR-181a was overexpressed through a mimic transfection, the LPS-induced inflammatory response was alleviated. The authors found that overexpression of miR-181a reduced the LPS-induced intracellular ROS accumulation, similarly to what happened by siTLR4 transfection. Finally, this study suggested that miR-181a could reduce LPS-induced inflammation by targeting TLR4 and subsequently reduce ROS accumulation [Id., citing Jiang, K. et al. Front. Pharmacol. (2018) 9: 142].

miR Let7: miR-let-7 family clusters demonstrate an anti-fibrotic role in lung fibrosis [Srivastava, S P et al., Front. Pharmacol. (2019) 10: 904, citing Pandit, K V et al., Am. J. Respir. Crit. Care Med. (2010) 182: 220-29; Rajasekaran, S. et al. Front. Pharmacol. (2015) 6: 254), cardiac fibrosis (Id., citing Wang, X. et al. Hypertension (2015) 66: 776-785), and renal fibrosis (Id., citing Brennan, E P et al. J. Am. Soc. Nephrol. (2013) 24: 627-37; Srivastava, S P et al. Fibrogenesis Tissue Repair (2014) 7: 12; Srivastava, S P et al. Sci. Rep. (2016) 6: 29884). It was shown that TGFβ 1 reinforces its signaling by mitigating miR-let-7b production, which targets the 3′UTR of TGFβR1 mRNA in rat tubule epithelial cells (TECs) (Id., citing Wang, B. et al. Kidney Int. (2014) 85: 352-61). Down-regulated miR-let-7b expression was found in mouse models of diabetic (Id., citing Nagai, T. et al. (2014) Biomed. Res. Int. (2014) 696475) and non-diabetic renal fibrosis (Id., citing Brennan et al., 2013). Similarly, miR-let-7c targets TGFβR1, collagen type 1 alpha 1 (COL1A1), collagen type 1 alpha 2 (COL1A2), and thrombospondin in human TECs (Id., citing Brennan, E P et al. J. Am. Soc. Nephrol. (2013) 24: 627-37). Lipoxins, which are endogenously produced lipid mediators, decrease renal fibrosis in a UUO model in the rats by elevating miR-let-7c expression (Id., citing Brennan, E P et al. J. Am. Soc. Nephrol. (2013) 24: 627-37), promote the resolution of inflammation, and inhibit fibrosis in cultured human proximal tubular epithelial (HK-2) cells ((Id., citing Brennan, E P et al. J. Am. Soc. Nephrol. (2013) 24: 627-37)). Lipoxin A4 (LXA4) has been shown to decrease TGFβ1-induced expression of mesenchymal markers, i.e., fibronectin, N-cadherin, thrombospondin, and the notch ligand jagged-1 in HK-2 cells through a mechanism by inducing of miR-let-7c (Id., citing Brennan, E P et al. J. Am. Soc. Nephrol. (2013) 24: 627-37)). In the UUO model of renal fibrosis, the expression level of miR-let-7c was up-regulated by treatment with LXA4 analog. LXA4 treatment caused up-regulation of miR-let-7c and inhibited TGFβR1 and its associated signaling. Therefore, LXA4-associated up-regulation of miR-let-7c expression suppresses TGFβ1-induced fibrosis, which is a key pathway that is dysregulated in human renal fibrosis.

miR146a: an NF-κB-associated gene [Lee, H-M et al. BMB Rep. (2016) 49 (6): 311-18, citing Taganov, K D et al. Proc. Natl Acad. Sci. USA (2006) 103: 12481-86] plays a role in negative regulation of the production of proinflammatory cytokines, thus modulating the severity of the inflammatory response [Id., citing Perry, M M et al. J. Immunol. (2008) 180: 5689-98]. MiR-146a plays a critical role in regulating the proliferation of immune cells and inhibiting inflammatory responses [Id., citing Boldin, M P et al. J. Exp. Med. (2011) 208: 1189-1201; Zhao, J L et al. Proc. Natl Acad. Sci. USA (2011) 108: 9184-9]. An miR-146a deficiency in mice is associated with chronic dysregulation of NF-κB signaling, yielding a phenotype with characteristics of myeloid malignancy [Id., citing Zhao, J L et al. Proc. Natl Acad. Sci. USA (2011) 108: 9184-9]. Both miR-146a and miR-146b can regulate inflammatory responses by targeting mRNAs encoding IRAK-1 and TRAF6 [Id., citing Taganov, K D et al. Proc. Natl. Acad. Sci. USA (2006) 103: 12481-6; Saba, R. et al. Front. Immunol. (2014) 5: 578; Cui, J G et al. J. Biol. Chem. (2010) 285: 38951-60; Park, H. et al. J. Biol. Chem. (2015) 290: 2831-41]. miR-146a plays an important role in the expression of tight junction proteins claudin-1 and JAM-A, suggesting that miR-146a is essential to the maintenance of tight junction barrier and innate immune defense [Id., citing Miyata, R. et al. Eur. J. Pharmacol. (2015) 761: 375-82]. In primary human keratinocytes, miR-146a can inhibit the development of NF-κB-dependent inflammatory responses by directly targeting recruitment (by the upstream nuclear factor kappa B) of the following three signal transducers: caspase domain-containing protein 10, IL-1 receptor-associated kinase 1, and chemokine (C—C motif) ligand (CCL) 5 [Id., citing Rebane, A. et al. J. Allergy Clin. Immunol. (2014) 134 (3811): 836-47]. Moreover, TLR2 stimulation can trigger sustained expression of miR-146a, which in turn will suppress the synthesis of IL-8, CCL20, and TNF-α in primary human keratinocytes [Id., citing Meisgen, F. et al. J. Invest. Dermatol. (2014) 134: 1931-40]. In addition, activation of TLR4 signaling can upregulate miR-146b expression in human monocytes via the action of IL-10-mediated STAT3-dependent pathway [Id., citing Curtale, G. et al. Proc. Nat. Acad. Sci. USA (2013) 110: 11499-504]. In turn, miR-146b can negatively regulate LPS-mediated production of many proinflammatory cytokines and chemokines. MiR-146b fulfills these roles by targeting many components of signaling pathways, including TLR4, MyD88, IRAK-1, and TRAF6[Id., citing Curtale, G. et al. Proc. Nat. Acad. Sci. USA (2013) 110: 11499-504].

miR 199: The regulatory effects of miR-199a are diverse. A large number of studies have indicated that the two mature types of miR-199a regulate the activities of normal cells to participate in corresponding physiological or pathological processes. For example, in the lung, expression of miR-199a-5p is upregulated by caveolin-1, which promotes lung fibroblast proliferation and differentiation; the high expression of miR0199s-5p promotes the formation of pulmonary fibrosis through the activation of the TGF-β signaling pathway by Caveolin-1. [Wang, Q. et al., Cancer Management & Res. (2019) 11: 10327-35, citing Lino, Cardenas, C L et al. PLoS Genet. (2013) 9: e1003291. Past studies have shown that miR-199a can induce apoptosis, either by upregulating the level of pro-apoptotic protein or decreasing the expression of anti-apoptotic protein in most situations. miR-199a-3p has been reported to cause more pronounced apoptosis than miR-199a-5p in cancer cells; in A549 cells, the apoptosis pathway induced by miR-199a-5p is caspase-dependent, whereas that induced by miR-199a-3p is caspase independent. [Id., citing Kim, S. et al. J. Biol. Chem. (2008) 283: 18158-66]. However, in some cases, miR-199a is involved in the anti-apoptosis effect; one study reported that miR-199a-5p is down-regulated and apoptosis is increased on a decline in oxygen tension of cardiac myocytes. Dual-luciferase reporting system assay revealed that HIF-1α is targeted gene of miR-144-5p. The results also showed that Sirt1 is a direct target of miR-199a-5p and is responsible for down-regulating prolyl hydroxylase 2, which is required for stabilization of HIF-1α. These results indicate that miR-199a can inhibit cardiomyocyte apoptosis under hypoxic conditions. [Id., citing Rane, S. et al. Cir. Res. (2009) 104: 879-86]. Other results demonstrate that overexpression of miR-199a-3p suppresses the p53/miR-3p/suppressor of cytokine signaling 7 (SOCS7) pathway, which suppresses SOCS7 signaling for STAT3 activation and renal fibrosis. [Id., citing Yang, R. et al. Sci. Rep. UK (2017) 7: 43409], Overexpression of miR-199a-5p also can impair autophagy and activate the mTOR/GSK3β signaling pathway, inhibit the activity of proteins, such as Atg5, Atg12, BECN1, and LCB3; and induce cardiac hypertrophy in mice. [Id., citing Id., citing Li, Z. et al. Cell Death Differ. (2017) 14: 1205-13].

In one study, miR34a and miR-199a-5p were overexpressed in the lungs of 55 COPD patients compared to histologically healthy lungs. In vitro studies and analysis of COPD lung tissues showed that miR-199a-5p was associated with hypoxia-inducible factor-la (HIF-1α) expression. The authors further investigated the relationship between oxidative stress/miR-34a/miR-199a-5p in COPD and suggested that oxidative stress induces miR-34 upregulation through the upregulation of p53. MiR-34 inhibited the activation and phosphorylation of AKT, which conversely caused miR-199a-5p upregulation. Finally, miR-199a-5p reduced the expression of HIF-1α, which can impair the VEGF expression that together with AKT inaction leads to cell apoptosis and emphysema. One caveat of the study was that more than 40% of the patients analyzed had lung cancer as well. [Climent, M. et al. Intl J. Mol. Sci. (2020) 21: 4370, citing Mizuno, S. et al. Chest (2012) 142: 64: 151-60].

miR 145 MiR-145, an miRNA known to regulate cancer and avascular smooth muscle cell phenotype [Climent, M. et al. Intl J. Mol. Sci. (2020) 21: 4370, citing Hu, H. et al. Lung Cancer (2016) 97: 87-94; Climent, M. et al. Cir. Res. (2015) 116: 1753-64] significantly lowered intracellular calcium and suppressed H2O2-mediated calcium overload in rat ventricular cardiomyocytes [Id., citing Cha, M. et al. Biochem. Biophys. Res. Commun. (2013) 435: 720-26]. MiR-145 also targeted Bcl2/adenovirus E1B 19 kDa-interacting protein 3 (Bnip3), which plays a critical function in the mitochondria, i.e., mediating apoptosis and sensing oxidative stress in the cytoplasm. Downregulation of Bnip3 by miR-145 was reported to cause a reduction in ROS production, showing a miR-145 protective role in cardiomyocytes undergoing oxidative stress as well as in the heart of mice subjected to I/R [Id., citing Li, R. et al. PLoS ONE (2012) 7: e44907].

It has been reported that miR-145 expression is upregulated in TGF-β1-treated lung fibroblasts in vitro and that miR-145 expression is also increased in the lungs of patients with idiopathic pulmonary fibrosis as compared to in normal human lungs. Overexpression of miR-145 in lung fibroblasts increased SMA-α expression, enhanced contractility, and promoted formation of focal and fibrillar adhesions. In contrast, miR-145 deficiency diminished TGF-β1 induced SMA-α expression. miR-145 did not affect the activity of TGF-β1, but promoted the activation of latent TGF-β1. miR-145 targets KLF4, a known negative regulator of SMA-α expression. miR-145−/− mice are protected from bleomycin-induced pulmonary fibrosis. [Yang, S. et al. FASEB J. (2013) 27 (6): 2382-91]. miR-21:

MiR-21 is a highly expressed miRNA in mammalian cells and is associated with different types of cancer. Several studies have reported a major contribution of miR-21 to apoptosis in both heart and lung tissues in oxidative stress.

In pulmonary vascular smooth muscle cells (VSMCs) undergoing oxidative stress, miR-21 has been reported to target PDCD4, exerting a protective role as it does in cardiac myocytes [Climent, M. et al. Int. J. Mol Sci. (2020) 21: 4370, citing Cheng, Y. et al. J. Mol. Cell Cardio. (2009) 47: 5-14] and human aortic endothelial cells (HAECs) [Id., citing Rippe, C. et al. Exp. Gerontol. (2012) 47: 45-51]. In the lung, chronic hypoxia causes a massive ROS production leading to pulmonary oxidative stress, which results in pulmonary vascular remodeling [Id., citing Araneda, O F and Tuesta, M. Oxid. Med. Cell Longev. (2012) 2012; Jiang, C. et al. Allergy Asthma Clin. Immunol. (2019) 15: 33]. Sarkar et al. found that hypoxia could induce the proliferation of pulmonary arterial smooth muscle cells (PASMCs) through the upregulation of miR-21 [Id., citing Sarkar, J. et al. Am. J. Physiol. Lung Cell Mol. Physio. (2010) 299: 861-71]. Therefore, miR-21 was upregulated and actively participated in ROS response during pulmonary remodeling [Id., citing Jiang, C. et al. Allergy Asthma Clin. Immunol. (2019) 15: 33].

High miR-21 levels are a marker of immune cell activation in multiple contexts, although whether or not this reflects a cause or consequence of activation remains to be determined. (Sheedy, F J. Front. Immunology (2015) 6: article 19). miR-21 expression is RNA polymerase II-dependent and derived from a primary transcript that is both capped and polyadenylated (Id., citing Cai, X. RNA (2004) 10: 1957-66). Similar to regular coding mRNAs, miR-21 expression is dynamically regulated by complex signaling pathways and can be enhanced by extracellular signals during immune cell development. Monocyte activation with phorbol 12-myristate 13-acetate (PMA, also known as 12-O-tetradecanoylphorbol-13-acetate or TPA)(Id., citing Kassashima, K. et al. Biochem. Biophys. Res. Comun. (2004) 322: 403-10) all trans retinoic acid to generate neutrophils (Id., citing Lu, J et al. Nature (2005) 435: 834-8), GM-CSF/IL-4 treatment to generate immature DCs (Id., citing Cekiaite, L. et al. Front. Biosci (Elite Ed.) (2010) 2: 818-28; Hashimi, S T et al. Blood (2009) 114: 404-14) treatment with LPS to generate activated macrophages (Id., citing Sheedy, F J et al. Nat. Immunol. (2010) 11: 141-7; Lu, Tex. et al. J. Immunol. (2010) 11: 141-7), and LPS-mediated B-cell activation (Id., citing 3), all revealed significant upregulation of miR-21. MiR-21 exhibits diversity in the signals, transcription factors and proposed binding sites that regulate its expression in diverse contexts. The complexity of the predicted promoter region of primary miRNA transcript (pri-miR-21) (Id., citing Fujita, S. et al. J. Mol. Biol. (2008) 378: 492-504; Loffler, D. et al. Blood (2007) 110: 1330-3) and the occurrence of alternative transcription start sites (Id., citing Ribas, J. et al. Nucleic Acids Res. (2012) 40: 6821-33) suggest that the regulation of miR-21 transcription is not straight forward.

miR-21 expression is upregulated by IL-6 and toll-receptor signaling, which activate STAT3 [Yang, C H et al. Pharmaceuticals (2015) 8: 836-847, citing Folini, M. et al. Mol. Cancer (2010) 9: 12; Loffler, D. et al. Blood (2007) 110: 1330-3]. Several lines of evidence indicate that both STAT3 and NFκB signaling pathways regulate miR-21 expression. Type I-interferon (IFN) induces expression of miR-21; this IFN-induction is STAT3-dependent.

miR-21 seems to be strongly associated with renal pathogenesis both in the glomerulus and tubulointerstitium of the kidney. [Ichi, O. and Horino, T. J. Toxicol. Pathol. (2018) 31: 23-34]. miR-21 targets several molecules including P53, PDCD4, SMAD7, TEGBR2, TIMP3, CDC25A, CDK6, ERK/MAPK, PTEN, PPARA, MPV17L, DDAHI and RECK. [Id.]

miRNA Sorting into Exosomes

miRNAs are not randomly incorporated into exosomes. Guduric-Fuchs et al. analyzed miRNA expression levels in a variety of cell lines and their respective derived exosomes, and found that a subset of miRNAs (e.g., miR-150, miR-142-3p, and miR-451) preferentially enter exosomes [Zhang, J. et al. Genomics Proteomics Bioinformatics (2015) 13: 17-24, citing Guduric-Fuchs J., et al. BMC Genomics. 2012; 13:357]. Similarly, Ohshima et al. compared the expression levels of let-7 miRNA family members in exosomes derived from the gastric cancer cell line AZ-P7a with those from other cancer cell lines, including the lung cancer cell line SBC-3/DMS-35/NCI-H69, the colorectal cancer cell line SW480/SW620, and the stomach cancer cell line AZ-521. As a result, they found that members of the let-7 miRNA family are abundant in exosomes derived from AZ-P7a, but are less abundant in exosomes derived from other cancer cells [Id., citing Ohshima K., et al. PLoS One. 2010; 5:e13247]. Moreover, some reports have shown that exosomal miRNA expression levels are altered under different physiological conditions. The level of miR-21 was lower in exosomes from the serum of healthy donors than those glioblastoma patients [Id., citing Skog J., et al. Nat Cell Biol. 2008; 10:1470-1476]. Levels of let-7f, miR-20b, and miR-30e-3p were lower in vesicles from the plasma of non-small-cell lung carcinoma patients than normal controls [Id., citing Silva J., et al. Eur Respir J. 2011; 37:617-623]. Different levels of eight exosomal miRNAs, including miR-21 and miR141, were also found between benign tumors and ovarian cancers [Id., citing Taylor D. D., et al. Gynecol Oncol. 2008; 110:13-21].

There are four potential modes for sorting of miRNAs into exosomes, although the underlying mechanisms remain largely unclear. These include: 1) The neural sphingomyelinase 2 (nSMase2)-dependent pathway. nSMase2 is the first molecule reported to be related to miRNA secretion into exosomes. Kosaka et al. found that overexpression of nSMase2 increased the number of exosomal miRNAs, and conversely inhibition of nSMase2 expression reduced the number of exosomal miRNAs [Id., citing Kosaka N., et al. J Biol Chem. 2013; 288:10849-10859]. 2) The miRNA motif and sumoylated heterogeneous nuclear ribonucleoproteins (hnRNPs)-dependent pathway. Villarroya-Beltri et al. discovered that sumoylated hnRNPA2B1 could recognize the GGAG motif in the 3′ portion of miRNA sequences and cause specific miRNAs to be packed into exosomes [Id., citing Villarroya-Beltri C., et al. Nat Commun. 2013; 4:2980]. Similarly, another two hnRNP family proteins, hnRNPA1 and hnRNPC, can also bind to exosomal miRNAs, suggesting that they might be candidates for miRNA sorting as well. However, no binding motifs have been identified yet [Id., citing Villarroya-Beltri C., et al. Nat Commun. 2013; 4:2980]. 3) The 3′-end of the miRNA sequence-dependent pathway. Koppers-Lalic et al. discovered that the 3′ ends of uridylated endogenous miRNAs were mainly presented in exosomes derived from B cells or urine, whereas the 3′ ends of adenylated endogenous miRNAs were mainly presented in B cells [Id., citing Koppers-Lalic D., et al. Cell Rep. 2014; 8:1649-1658]. The above two selection modes commonly indicate that the 3′ portion or the 3′ end of the miRNA sequence contains a critical sorting signal. 4) The miRNA induced silencing complex (miRISC)-related pathway. It is well known that mature miRNAs can interact with assembly proteins to form a complex called miRISC. The main components of miRISC include miRNA, miRNA-repressible mRNA, GW182, and AGO2. The AGO2 protein in humans, which prefers to bind to U or A at the 5′ end of miRNAs, plays an important role in mediating mRNA:miRNA formation and the consequent translational repression or degradation of the mRNA molecule [Id., citing Frank F., et al. Nature. 2010; 465:818-822]. Recent studies recognized a possible correlation between AGO2 and exosomal miRNA sorting.

The miRNAs in cell-released exosomes can circulate with the associated vehicles to reach neighboring cells and distant cells. After being delivered into acceptor cells, exosomal miRNAs play functional roles. Although it is difficult to completely exclude the effects of other exosomal cargos on recipient cells, miRNAs are considered the key functional elements. The functions of exosomal miRNAs can be generally classified into two types. One is the conventional function, i.e., miRNAs perform negative regulation and confer characteristic changes in the expression levels of target genes. For example, exosomal miR-105 released from the breast cancer cell lines MCF-10A and MDA-MB-231 reduced ZO-1 gene expression in endothelial cells and promoted metastases to the lung and brain [Id., citing Zhou W., et al. Cancer Cell. 2014; 25:501-515]. Exosomal miR-214, derived from the human microvascular endothelial cell line HMEC-1, stimulated migration and angiogenesis in neighboring HMEC-1 cells [Id., citing van Balkom B. W., et al. Blood. 2013; 121:S1-S15]. Exosomal miR-92a, derived from K562 cells, significantly reduced the expression of integrin a5 in the human umbilical vein endothelial (HUVEC) cells and enhanced endothelial cell migration and tube formation [Id., citing Umezu T., et al. Oncogene. 2013; 32:2747-2755]. The other one is a novel function that has been identified in some miRNAs when they are studied as exosomal miRNAs rather than intracellular miRNAs. Exosomal miR-21 and miR-29a, in addition to the classic role of targeting mRNA, were first discovered to have the capacity to act as ligands that bind to toll-like receptors (TLRs) and activate immune cells [Id., citing Fabbri M., et al. Proc Natl Acad Sci USA. (2012) 109:E2110-E2116].

Exosomal miRNAs can stably exist in the blood, urine, and other body fluids of patients, and exosomes can reflect their tissue or cell of origin by the presence of specific surface proteins [Id., citing Simons M., et al. Curr Opin Cell Biol. 2009; 21:575-581, Mathivanan S., et al. J Proteomics. 2010; 73:1907-1920, Gross J. C., et al. Nat Cell Biol. 2012; 14:1036-1045]. Furthermore, the amount and composition of exosomal miRNAs differ between patients with disease and healthy individuals. Thus, exosomal miRNAs show potential for use as noninvasive biomarkers to indicate disease states. Several previous studies have profiled exosomal miRNAs in different samples. Some exosomal miRNAs can be used to aid in clinical diagnosis [Id., citing Skog J., et al. Nat Cell Biol. 2008; 10:1470-1476; Silva J., et al. Eur Respir J. 2011; 37:617-623; Taylor D. D., et al. Gynecol Oncol. 2008; 110:13-21; Rabinowits G., et al. Clin Lung Cancer. 2009; 10:42-46]. For example, a set of exosomal miRNAs, including let-7a, miR-1229, miR-1246, miR-150, miR-21, miR-223, and miR-23a, can be used as the diagnostic biomarker of colorectal cancer [Id., citing Ogata-Kawata H., et al. PLoS One. (2014) 9:e92921]. Another set, miR-1290 and miR-375, can be used as the prognostic marker in castration-resistant prostate cancer [Id., citing Huang X., et al. BMC Genomics. 2013; 14:319].

Besides endogenous miRNAs, exogenous miRNAs can also be sorted into exosomes, which has been experimentally confirmed by Pegtel et al. [Id., citing Pegtel D. M., et al. Proc Natl Acad Sci USA. 2010; 107:6328-6333] and Meckes et al. [Id., citing Meckes, D G, Jr. et al. Proc. Natl. Acad. Sci. USA (2010) 107: 20370-75], who observed that human tumor viruses can exploit exosomes as delivery vectors to transfer their exogenous miRNAs to other non-infected cells [Id., citing Pegtel D. M., et al. Proc Natl Acad Sci USA. 2010; 107:6328-6333, Meckes D. G., Jr., et al. Proc Natl Acad Sci USA. 2010; 107:20370-20375]. Hence, exogenous small RNAs have also been transferred by exosomes by mimicking the molecular mechanism of endogenous miRNAs transportation.

### DNA

Studies have shown that certain EVs may contain DNA fragments [Shao, H. et al. Chem Rev. (2018) 118 (4): 1917-50], citing Balaj, L. et al. Nat. Commun. (2011) 2: 180; Thakur, B K et al. Cell Res. (2014) 24: 766-9; Guescini, M. et al. J. Neural. Trans. (Vienna) (2010) 17: 1-4; Kahlert, C. et al. J. Biol. Chem. (2014) 289: 3869-75; Takahashi, A. et al. Nat. Commun. (2017) 8: 15287]. These DNA are double-stranded fragments which range from 100 base pairs (bp) to 2.5 kbp [Id., citing Thakur, B K et al. Cell Res. (2014) 24: 766-9]. The larger-sized population (>2.5 kbp) was found to be predominately external DNA associated with EVs and smaller-sized population (100 bp-2.5 kbp) as internal DNA confined within EVs. These fragments represent the whole genomic DNA and could be used to identify mutations present in parental tumor cells [Id., citing Thakur, B K, et al., Kahlert, C. et al. J. Biol. Chem. (2014) 289: 3869-75]. The functional roles of these DNAs have yet to be determined.

### EV Composition:

The heterogeneity of exosomes and or extracellular vesicles is thought to be reflective of their size, content, functional impact on recipient cells, and cellular origin. During their secretion they acquire surface proteins from their cell of origin. They naturally transport mRNA, miRNA, and proteins between cells.

Biofluids can contain large quantities of EVs that shuttle various molecules from parental cells to other cells, including proteins [Id., citing Graner, M W et al. FASEB J. (2009) 23: 1541-57; Simpson, R J et al. Proteomics (2009) 6: 267-83; Mathivanan, S. et al. Nucleic Acids, Res. (2012) 40: D1241-4], mRNA/miRNA [Id., citing Valadi, H. et al. Nat. Cell Biol. (2007) 9: 654-9; Skog, J. et al. Nat. Cell Biol. (2008) 10: 1470-6] and DNA [Id., citing Balaj, L. et al. Nat. Commun. (2011) 2: 180] Extracellular vesicles therefore are mediators of near and long-distance intercellular communication in health and disease and affect various aspects of cell biology.

EV composition is determined not only by the cell type but also by the physiological state of the producer cells. The diversity of mechanisms by which EVs are generated and confer effects provides both opportunities and challenges for developing EV-based therapeutics (Gyorgy B, et al. Annu Rev Pharmacol Toxicol. (2015) 55: 439-464). Many methods are used to isolate EVs, and EV contents and properties overlap with those of the cells of origin and other EV types. Formalizing EV nomenclature and defining attributes is a work in progress. The mechanisms of EV uptake and content delivery (or degradation) vary among EV types and recipient cell types. Elucidating and understanding these processes is critical for harnessing EVs as therapeutic delivery vehicles. Multiple lines of evidence indicate that EVs can transfer biomolecules to modulate recipient cell state in vivo, for example, following bolus injection of purified or concentrated EVs. However, the extent to which such processes naturally shape cellular function and intercellular communication, particularly under homeostatic conditions, remains poorly understood. Moreover, we do not understand the relative importance of EV-mediated transfer between proximal cells, for example, when diffusional barriers lead to local accumulation of secreted EVs rather than transfer of EVs via the circulation, where EV concentrations may be lower. EV-mediated signaling is dose-dependent (Id., citing Yu S, et al. J. Immunol. 2007; 178: 6867-75), so the tuning of EV dose may enable the balancing of potential deleterious and therapeutic effects of EV administration. Understanding the role of EV dose is also important for achieving therapeutic efficacy.

### EV Binding

EV binding is mediated by receptors that interact with either universal EV molecules, such as lipids and carbohydrates, or specific peptides present on subsets of EVs. Following initial binding, cells internalize EVs by processes that include receptor-mediated phagocytosis or endocytosis via receptors that include T cell immunoglobulin- and mucin-domain-containing molecule-4 (TIM4), which binds to phosphatidylserine (PS) on EVs; scavenger receptors; integrins; and complement receptors (Gyorgy B, et al. Annu Rev Pharmacol Toxicol. (2015) 55: 439-464, citing Record M, et al. Biochem. Pharmacol. 2011; 81: 1171-82). How EV cargo is released into the cytoplasm after entry into recipient cells is unclear. Furthermore, uptake of cargo into a cell is not equivalent to cargo functionality. For instance, EVs may potentially pass through cells within the multivesicular body compartment, which could explain how EVs cross the blood-brain barrier (BBB) (i.e., via a transendothelial route). Endocytotic mechanisms must circumvent the lysosomal degradative pathway, and direct fusion between the EV and target cell plasma membrane or endocytotic membrane does not always ensure functionality of the contents. In many cases, EV cargo can be degraded by recipient cells, thereby inhibiting therapeutic delivery but limiting the impact of off-target delivery. In general, the fate of EVs within the body and cells remains poorly understood and requires additional investigation to elucidate how these processes impact functional EV-mediated delivery (Id.).

EVs comprising exosomes are released by most if not all cell types, including platelets, blood cells, dendritic cells, mast cells, T cells, B cells, epithelial cells, endothelial cells, mesenchymal stem cells, smooth muscle cells, neuronal cells and many tumor cells. [Zhang, J. et al. Genomics Proteomics Bioinformatics (2015) 13: 17-24, citing Liao J., et al. Int J Mol Sci. 2014; 15:15530-15551; Kopers-Lalic, D. et al. Adv. Drug delivery rev. (2012) doi: 10.1016/j.addr.2012.07.006, citing Thery V., et al. Nat. rev. Immunol. (2002) 2: 569-79].

Most EVs comprising exosomes share a core set of proteins and lipids; there seems to be a clear conserved protein repertoire in exosomes across cell-types and species [Kopers-Lalic, D. et al. Adv. Drug delivery rev. (2012) doi: 10.1016/j.addr.2012.07.006, citing Simpson, R J, et al. Proteomics (2008) 8: 4083-99]. For example the endosomal proteins such as Alix and TSG101 have been identified in the majority of the exosomes studied for their protein content thus far. In addition, heat shock proteins, which are involved in protein trafficking, are frequently found in exosomes [Id., citing van Dommelen, S M et al. J. Control. Release (2011) 161: 635-44]. Exosomes are further enriched in tetraspanins, like CD9, CD63, CD81 and CD82, which are important molecules for protein-protein interactions in cellular membranes. Tetraspanins bind many proteins, including integrins and MHC molecules [Id., citing Thery, C., et al. Nat. Rev. Immunol. (2009) 9: 581-93; Escola, J M. J. Biol. Chem. (1998) 273: 20121-27; Keller, S. et al. Immunol. Lett (2006) 107: 102-108; Stoorvogel, W. et al. Traffic (2002) 3: 321-330]. Specific Rab proteins, a highly conserved family of small GTPases that functional as molecular switches and coordinate membrane traffic [Id., citing Stenmark, H. Nat. Rev. Mol. Cell Biol. (2009) 10: 513-25], are often observed in exosomes by mass-spectrometry. Exosomes are also rich in annexins, membrane trafficking proteins that are involved in fusion events. Furthermore cytoskeletal proteins like myosin, actin and tubulin are present in exosomes. Finally, metabolic enzymes, antigen presentation molecules, ribosomal proteins and signal transduction molecules have been shown to be present in exosomes [Id., citing Mathivanan, S., et al. Proteomics (2008) 8: 4083-99].

Besides selected sets of proteins, EVs comprising exosomes also incorporate (functional) nucleic acids, most notably small RNA molecules [Id., citing Zomer, A. et al. Commun. Integr. Biol. (2010) 3: 447-50; Gibbings, D, Voinnet, O. Trends Cell Biol. (2010) 20: 491-501]. Of all RNA molecules detected in exosomes, the class of 22 nt long, non-coding miRNAs has received attention since the discovery that miRNAs can be functionally transferred to recipient cells [Id., citing Pegtel, D M et al. Proc. Nat. Acad. Sci. USA (2010) 107: 6328-33, Valadi, H. et al. Nat. Cell Biol. (2007) 9: 654-59]. MiRNAs regulate gene expression by binding imperfectly to the 3′ untranslated region of the target mRNA that results in translational repression of the mRNA into protein [Id., citing Bartel, D P. Cell (2004) 116: 281-97; Brennecke, J. et al. PLoS Biol. (2005) 3: e85].

EVs comprising exosomes also contain specific proteins depending on the cell of origin. As examples, exosomes from tumor cells contain tumor antigens, platelet-derived exosomes contain coagulation factors, and exosomes from dendritic cells express toll-like receptor ligands [Zhu, L. et al., Artificial Cells, nanomedicine and biotechnology (2018) 46 (53): S166-S179, citing Jong, A Y et al. J. Extracell. Vesicles (2017) 6: 1294368; Sobo-Vujanovic, A. et al., Cell Immunol. (2014) 289: 119-127]. Exosome-mediated functions vary depending on the condition or the origin of the cells [Id., citing Kim, O Y, et al. Semin. Cell Dev. Biol. (2017) 67: 74-82].

### Trafficking of Exosomes

There are three mechanisms of interaction between EVs comprising exosomes and their recipient cells. First, the transmembrane proteins of exosomes directly interact with the signaling receptors of target cells [Zhang, J. et al. Genomics Proteomics Bioinformatics (2015) 13: 17-24, citing Munich S., Sobo-Vujanovic A., Buchser W. J., Beer-Stolz D., Vujanovic N. L. Dendritic cell exosomes directly kill tumor cells and activate natural killer cells via TNF superfamily ligands. Oncoimmunology. 2012; 1:1074-1083]. Second, the exosomes fuse with the plasma membrane of recipient cells and deliver their content into the cytosol [Id., citing Mulcahy L. A., et al. J Extracell Vesicles. (2014) 3: 10.3402/jev/v3/24641]. Third, the exosomes are internalized into the recipient cells and have two fates. In one case, some engulfed EVs comprising exosomes may merge into endosomes and undergo transcytosis, which will move EVs comprising exosomes across the recipient cells and release them into neighboring cells. In the other case, endosomes fused from engulfed exosomes will mature into lysosomes and undergo degradation [Id., citing Mulcahy L. A., et al. J Extracell Vesicles. (2014) 3: 10.3402/jev/v3/24641, Tian T., et al. J Cell Physiol. 2013; 228:1487-1495]. Some factors influencing internalization of exosomes in recipient cells have been reported. For example, Koumangoye et al. observed that disruption of exosomal lipid rafts resulted in the inhibition of internalization of exosomes and that annexins, which are related to cell adhesion and growth, were essential for the uptake of exosomes in the breast carcinoma cell line BT-549 [Id., citing Koumangoye R. B., et al. PLoS One. 2011; 6:e24234]. Escrevente et al. described a decrease in exosome uptake after the ovarian carcinoma cell line SKOV3 and its derived exosomes were treated with protease K, which indicated that the proteins mediating exosome internalization are presented on the surface of both the cells and the exosomes [Escrevente C., et al. BMC Cancer. 2011; 11:108].

### Exosome Function

EVs comprising exosomes exert their functions through a number of different mechanisms. They can transfer their cargo or membrane constituents from one cell to another, thus transferring functions between cells. [Mathieu, M. et al. Nat. Cell Biol. (2019) 21 (10): 9-17]. They carry molecules on their surface, which can act as ligands that stimulate surface receptors in other cells activating intracellular signaling [Maia, J. et al. Front. Cell Dev. Biol. (2018) 6: 18]. These surface molecules can directly perform functions in the extracellular milieu; for example, exosomes may carry remodeling enzymes of their surface, such as matrix metalloproteinases, heparanases, and hyaluronidases. [Nawaz, M. et al. Cells (2018) 7 (10) PMC6210724]. Surface proteins on the EV membrane also may capture external molecules or pathogens to neutralize their effects.

It is believed that exosomes can regulate the bioactivities of recipient cells by the transportation of lipids, proteins, and nucleic acids while circulating in the extracellular space. [Zhang, J. et al. Genomics Proteomics Bioinformatics (2015) 13: 17-24, citing Liao J., et al. Int J Mol Sci. 2014; 15:15530-15551].

### EVs Play a Role in Several Normal and Disease Physiological Processes.

### Blood Hemostasis.

Molecules on the surface of some EVs, such as tissue factor or phosphatidylserine, work as activators of the coagulation cascade. These EVs, which are available in the blood during injury or endothelial damage are absent from healthy circulating blood. Normal circulating EVs carry plasminogen activators, which induce fibrinolytic activity, preventing thrombus formation. Additionally, exosomes released from platelets under normal conditions inhibit platelet aggregation. (Zarra, M. et al. Intl J. Mol. Sci. (2019) 20 (11 PMX6600675).

### Wound Healing.

With regard to the role of miRNAs in the proliferation and differentiation of MSCs in the setting of wound healing (Guo, L. et al. Exptl Hematol. (2011) 39: 608-616, citing Silo, S., et al. DNA Cell Biol. (2007) 26: 227-37), using a skin excision model, altered expression in a panel of miRNAs, including upregulated expression of miR-31, -21, -223, -142, -205, -203, -18b, -19a, -130b, -16, -26b, -125b, and let-7f, and down regulated expression of miR-133a, -181, -30a-3p, -193b, -30a-5p, -204, -200b, -96, -127, -181c, -182 and -130a was demonstrated at the stage of active granulation formation (Id., citing Zou, Z. et al. Expert Opin. Biol. Thera. (2010) 10: 215-30). Further, TGFβ, a key growth factor elevated in the wound site, was found to stimulate upregulation of miR-21 in MSCs as well as in multipotential C3H10T1/2 cells, and to promote proliferation and differentiation of these cells in vitro. [Zou, Z. et al., Expert Opin. Biol. Thera. (2010) 10: 215-30]. Consistently, knockdown of miR-21 in the wound bed delayed the healing process. These results suggest that miR-21 regulates gene expression and, subsequently, the behavior of MSCs in wound healing.

Exosomes derived from bone mesenchymal stem cells preconditioned by stimulation with Fe₃O₄ nanoparticles and a static magnetic field (mag-BMSC-Exos) were reported to enhance wound healing through upregulated miR-21-5p. [Wu, D. et al. Intl J. Nanomedicine (2020) 15: 7979-93]. mag-BMSC-Exos were compared to exosomes derived from bone marrow mesenchymal stem cells (BMSC-exo) without preconditioning. Both were isolated by ultracentrifugation. Wound healing in in vitro experiments, including scratch wound assays, transwell assays, and tube formation assays, and an established an in vivo wound healing model were compared as were the miRNA expression profiles were compared between BMSC-Exos and mag-BMSC-Exos.

In vitro studies have shown that exosomes derived from human adipose mesenchymal stem cells (ASCs-Exos) could be taken up and internalized by fibroblasts to stimulate cell migration, proliferation and collagen synthesis in a dose-dependent manner, with increased genes expression of N-cadherin, cyclin-1, PCNA and collagen I, III. In vivo, tracing experiments demonstrated that ASC-Exos could be recruited to soft tissue wound area in a mouse skin incision model and that they significantly accelerated cutaneous wound healing. Hu, L. et al. Sci. rep. (2016) 6: 32993].

The efficacy of exosomes derived from human umbilical cord blood (UCB-Exos) on wound healing was evaluated by measuring wound closure rates, histological analysis and immunofluorescence examinations. In vitro, quantitative real-time PCR (qRT-PCR) analysis was performed to detect the expression levels of a class of miRNAs that have positive roles in regulating wound healing. The scratch wound assay, transwell assay and cell counting kit-8 analysis were conducted to assess the effects of UCB-Exos on migration and proliferation of human skin fibroblasts and endothelial cells. Tube formation assay was carried out to test the impact of UCB-Exos on angiogenic tube formation ability of endothelial cells. Meanwhile, by using specific RNA inhibitors or siRNAs, the roles of the candidate miRNA and its target genes in UCB-Exos-induced regulation of function of fibroblasts and endothelial cells were assessed.

In vitro, exosomes derived from human umbilical cord blood (UCB-Exos) could promote the proliferation and migration of fibroblasts, and enhance the angiogenic activities of endothelial cells. In vivo, the local transplantation of exosomes derived from human umbilical cord blood (UCB-Exos) into full thickness mouse skin wounds resulted in accelerated re-epithelialization, reduced scar widths, and enhanced angiogenesis. It was concluded that accelerated cutaneous wound healing following local transplantation of UCB-Exos occurred through miR-3p-mediated promotion of angiogenesis and fibroblast function. Hu, Y. et al. Theranostics (2018) 8 (10: 169-84).

### Metabolic Functions

EVs play essential metabolic functions by transferring enzymes and metabolites between cells or by performing extracellular metabolic activities. Studies have shown that EVs secreted by liver hepatocytes contain hundreds of enzymes that belong to the different metabolic pathways. When incubated with rat serum, these ECs changed the metabolic profile of the serum. [Royo, F. et al., body metabolism. [Royo, F. et al. Sci. Rep. (2017) 7: 42798].

### Fibrosis as a Pathology

Fibrosis represents the formation or development of excess fibrous connective tissue in an organ or tissue, which is formed as a consequence of the normal or abnormal/reactive wound healing response leading to a scar. Although the fibrogenic response may have adaptive features in the short term, when it progresses over a prolonged period of time, parenchymal scarring and ultimately cellular dysfunction and organ failure ensue [Rockey D C et al., N Engl J Med. (2015) 372(12): 1138-49]. Fibrosis is characterized by, for example, without limitation, an aberrant deposition of an extracellular matrix protein, an aberrant promotion of fibroblast proliferation, an aberrant induction of differentiation of a population of fibroblasts into a population of myofibroblasts, an aberrant promotion of attachment of myofibroblasts to an extracellular matrix, or a combination thereof.

There are four major phases of the fibrinogenic response. First is initiation of the response, driven by primary injury to the organ. The second phase is the activation of effector cells, and the third phase is the elaboration of extracellular matrix, both of which overlap with the fourth phase, during which the dynamic deposition (and insufficient resorption) of extracellular matrix promotes progression to fibrosis and ultimately to end-organ failure (Id.).

The fact that diverse diseases in different organ systems are associated with fibrotic changes suggests common pathogenic pathways (Id.). This “wounding response” is orchestrated by complex activities within different cells in which specific molecular pathways have emerged. Cellular constituents include inflammatory cells (e.g., macrophages and T cells), epithelial cells, fibrogenic effector cells, endothelial cells, and others. Many different effector cells, including fibroblasts, myofibroblasts, cells derived from bone marrow, fibrocytes, and possibly cells derived from epithelial tissues (epithelial-to-mesenchymal transition) have been identified; there is some controversy regarding the identity of specific effectors in different organs. Beyond the multiple cells essential in the wounding response, core molecular pathways are critical; for example, the transforming growth factor beta (TGF-β) pathway is important in virtually all types of fibrosis (Id.).

As fibrosis progresses, myofibroblasts proliferate and sense physical and biochemical stimuli in the local environment by means of integrins and cell-surface molecules; contractile mediators trigger pathological tissue contraction. This chain of events, in turn, causes physical organ deformation, which impairs organ function. Thus, the biology of fibrogenesis is dynamic, although the degree of plasticity appears to vary from organ to organ (Id.).

Acute and chronic inflammation often trigger fibrosis (Id.). Inflammation leads to injury of resident epithelial cells and often endothelial cells, resulting in enhanced release of inflammatory mediators, including cytokines, chemokines, and others. This process leads to the recruitment of a wide range of inflammatory cells, including lymphocytes, polymorphonuclear leukocytes, eosinophils, basophils, mast cells, and macrophages. These inflammatory cells elicit the activation of effector cells which drive the fibrogenic process (Id., citing Wynn T A. Nat Rev Immunol 2004; 4: 583-94). In addition, macrophages can play a prominent role in interstitial fibrosis, often driven by the TGF-β pathway (Id., citing Meng X M, et al. Nat Rev Nephrol 2014; 10: 493-503). However, some inflammatory cells may be protective. For example, certain populations of macrophages phagocytose apoptotic cells that promote the fibrogenic process and activate matrix-degrading metalloproteases (Id., citing Ramachandran P, Iredale J P. J Hepatol 2012; 56: 1417-9). Fibroblasts and myofibroblasts have been identified as key fibrosis effectors in many organs, and as such are responsible for the synthesis of extracellular matrix proteins (Id., citing Hinz B, et al. Am J Pathol 2007; 170: 1807-16).

The matrix proteins that compose the fibrotic scar, which are highly conserved across tissues, consist predominantly of interstitial collagens (types I and III), cellular fibronectin, basement-membrane proteins such as laminin, and other, less abundant elements. In addition, myofibroblasts, which by definition are cells that express smooth-muscle proteins, including actin (ACTA2), are contractile (Id., citing Rockey D C, et al. J Clin Invest 1993; 92: 1795-804). The contraction of these cells contributes to the distortion of parenchymal architecture, which can promote disease pathogenesis and tissue failure. However, myofibroblasts also contribute to the normal wound healing process by contracting the edges of the wound and synthesizing and depositing extracellular matrix components (Hinz B. Curr Res Transl Med. 2016 October-December; 64(4): 171-177; Darby I A, et al. Clin Cosmet Investig Dermatol. 2014; 7: 301-311).

### Pro-Inflammatory Mediators

Accumulating evidence has suggested that polypeptide mediators known as cytokines, including various lymphokines, interleukins, and chemokines, are important stimuli to collagen deposition in fibrosis. Released by resident tissue cells and recruited inflammatory cells, cytokines are thought to stimulate fibroblast proliferation and increased synthesis of extracellular matrix proteins, including collagen. For example, an early feature in the pathogenesis of idiopathic pulmonary fibrosis is alveolar epithelial and/or capillary cell injury. This promotes recruitment into the lung of circulating immune cells, such as monocytes, neutrophils, lymphocytes and eosinophils. These effector cells, together with resident lung cells, such as macrophages, alveolar epithelial and endothelial cells, then release cytokines, which stimulate target cells, typically fibroblasts, to replicate and synthesize increased amounts of collagen. Breakdown of extracellular matrix protein also may be inhibited, thereby contributing to the fibrotic process. (Coker and Laurent, Eur Respir J, 1998, 11: 1218-1221)

Numerous cytokines have been implicated in the pathogenesis of fibrosis, including, without limitation, transforming growth factor-β (TGF-β), tumor necrosis factor-α (TNF-α), platelet-derived growth factor (PDGF), insulin-like growth factor-1 (IGF-1), endothelin-1 (ET-1) and the interleukins, interleukin-1 (IL-1), interleukin-6 (IL-6), interleukin-8 (IL-8), and interleukin-17 (IL-17). Chemokine leukocyte chemoattractants, including the factor Regulated upon Activation in Normal T-cells, Expressed and Secreted (RANTES), are also thought to play an important role. Elevated levels of pro-inflammatory cytokines, such as Interleukin 8 (IL-8), as well as related downstream cell adhesion molecules (CAMs) such as intercellular adhesion molecule-1 (ICAM-1) and vascular cell adhesion molecule-1 (VCAM-1), matrix metalloproteinases such as matrix metalloproteinase-7 (MMP-7), and signaling molecules such as S100 calcium-binding protein A12 (S100A12, also known as calgranulin C), in the peripheral blood have been found to be associated with mortality, lung transplant-free survival, and disease progression in patients with IPF (Richards et al, Am J Respir Crit Care Med, 2012, 185: 67-76).

The molecular processes driving fibrosis are wide-ranging and complex. The TGF-β cascade, which plays a major role in fibrosis, involves the binding of a ligand to a serine-threonine kinase type II receptor that recruits and phosphorylates a type I receptor. This type I receptor subsequently phosphorylates SMADs, which function as downstream effectors, typically by modulating target gene expression. TGF-β, which is a potent stimulator of the synthesis of extracellular matrix proteins in most fibrogenic cells, and is synthesized and secreted by inflammatory cells and by effector cells, thereby functioning in both an autocrine and paracrine fashion (Id.). The TGF-β family of proteins has a potent stimulatory effect on extracellular matrix deposition, and in fact has been used in constructing induced animal models of fibrosis through gene transfer. In vitro studies show that TGF-β1, secreted as a latent precursor, promotes fibroblast procollagen gene expression and protein synthesis. The data suggest that the other mammalian isoforms, TGF-β2 and TGF-β3, also stimulate human lung fibroblast collagen synthesis and reduce breakdown in vitro. In animal models of pulmonary fibrosis, enhanced TGF-β1 gene expression is temporally and spatially related to increased collagen gene expression and protein deposition. TGF-β1 antibodies reduce collagen deposition in murine bleomycin-induced lung fibrosis, and human fibrotic lung tissue shows enhanced TGF-β1 gene and protein expression.

TNF-α can stimulate fibroblast replication and collagen synthesis in vitro, and pulmonary TNF-α gene expression rises after administration of bleomycin in mice. Soluble TNF-α receptors reduce lung fibrosis in murine models, and pulmonary overexpression of TNF-α in transgenic mice is characterized by lung fibrosis. In patients with IPF or asbestosis (a chronic inflammatory and fibrotic medical condition affecting the parenchymal tissue of the lungs caused by the inhalation and retention of asbestos fibers), bronchoalveolar lavage fluid-derived macrophages release increased amounts of TNF-α compared with controls.

Platelet-derived growth factor (PDGF), connective-tissue growth factor (CTGF), and vasoactive peptide systems (especially angiotensin II and endothelin-1) play important roles (Id., citing Wynn T A. J Clin Invest 2007; 117: 524-9). Among vasoactive systems, endothelin plays a role in fibrosis in virtually all organ systems, acting through G-protein-coupled endothelin-A or endothelin-B cell-surface receptors or both (Id., citing Khimji A K, Rockey D C. Cell Signal 2010; 22: 1615-25). Endothelin (ET-1) also fulfills the criteria for a profibrotic cytokine. This molecule promotes fibroblast proliferation and chemotaxis and stimulates procollagen production. It is present in the lungs of patients with pulmonary fibrosis, and a recent report suggests that bosentan, an ET-1 receptor antagonist, ameliorates lung fibrosis when administered to experimental animals. Furthermore, angiogenic pathways may be important in fibrosis (Id., citing Johnson A, DiPietro L A. FASEB J 2013; 27: 3893-901). Integrins, which link extracellular matrix to cells, are considered critical in the pathogenesis of fibrosis (Id., citing Levine D, et al. Am J Pathol 2000; 156: 1927-35; Henderson N C, et al. Nat Med 2013; 19: 1617-24).

### Regenerative Cells of the Lungs

The lung is a highly quiescent tissue, previously thought to have limited reparative capacity and a susceptibility to scarring. It is now known that the lung has a remarkable reparative capacity, when needed, and scarring or fibrosis after lung injury may occur infrequently in scenarios where this regenerative potential is disrupted or limited (Kotten, D. N. and Morrisey, E. E., “Lung regeneration: mechanisms, applications and emerging stem cell populations,” Nat. Med. (2014) 20(8): 822-32, citing Beers, M F and Morrisey, E E, “The 3 R's of lung health and disease—repair, remodeling and regeneration,” J. Clin. Invest. (2011) 121: 2065-73; and Wansleeben, C. et al, “Stem cells of the adult lung: their development and role in homeostasis, regeneration and disease,” Wiley Interdiscip. Rev. Dev. Biol. (2013) 2: 131-148). Thus, the tissues of the lung may be categorized as having facultative progenitor cell populations that can be induced to proliferate in response to injury as well as to differentiate into one or more cell types.

The adult lung comprises at least 40-60 different cell types of endodermal, mesodermal, and ectodermal origin, which are precisely organized in an elaborate 3D structure with regional diversity along the proximal-distal axis. In addition to the variety of epithelial cells, these include cartilaginous cells of the upper airways, airway smooth muscle cells, interstitial fibroblasts, myofibroblasts, lipofibroblasts, and pericytes as well as vascular, microvascular, and lymphatic endothelial cells, and innervating neural cells. The regenerative ability of lung epithelial stem/progenitor cells in the different regions of the lung are thought to be determined not only by their intrinsic developmental potential but also by the complex interplay of permissive or restrictive cues provided by these intimately associated cell lineages as well as the circulating cells, soluble and insoluble factors and cytokines within their niche microenvironment (McQualter & Bertoncello., Stem Cells. 2012 May; 30(5); 811-16).

The crosstalk between the different cell lineages is reciprocal, multidirectional, and interdependent. Autocrine and paracrine factors elaborated by mesenchymal and endothelial cells are required for lung epithelial cell proliferation and differentiation (Yamamoto et al., Dev Biol. 2007 August 1; 308(1): 44-53; Ding et al., Cell. 2011 Oct. 28; 147(3): 539-53), while endothelial and epithelial cell-derived factors also regulate mesenchymal cell proliferation and differentiation, extracellular matrix deposition and remodeling, and adhesion-mediated signaling (Crivellato. Int J Dev Biol. 2011; 55(4-5): 365-75; Grinnell & Harrington. Pulmonary endothelial cell interactions with the extracellular matrix. In: Voelkel N F, Rounds S, eds. The Pulmonary Endothelium: Function in Health and Disease. Chichester, West Sussex: Wiley-Blackwell, 2009: 51-72). Chemotactic factors elaborated by these cell lineages also orchestrate the recruitment of inflammatory cells, which participate in the remodeling of the niche and the regulation of the proliferation and differentiation of its cellular constituents (McQualter & Bertoncello. Stem Cells. 2012 May; 30(5); 811-16).

### Stem Cell Niches

Adult tissue compartments contain endogenous niches of adult stem cells that are capable of differentiating into diverse cell lineages of determined endodermal, mesodermal or ectodermal fate depending on their location in the body. For example, in the presence of an appropriate set of internal and external signals, bone marrow-derived adult hematopoietic stem cells (HSCs) have the potential to differentiate into blood, endothelial, hepatic and muscle cells; brain-derived neural stem cells (NSCs) have the potential to differentiate into neurons, astrocytes, oligodendrocytes and blood cells; gut- and epidermis-derived adult epithelial stem cells (EpSCs) have the potential to give rise to cells of the epithelial crypts and epidermal layers; adipose-derived stem cells (ASCs) have the potential to give rise to fat, muscle, cartilage, endothelial cells, neuron-like cells and osteoblasts; and bone-marrow-derived adult mesenchymal stem cells (MSCs) have the potential to give rise to bone, cartilage, tendon, adipose, muscle, marrow stroma and neural cells.

Endogenous adult stem cells are embedded within the ECM component of a given tissue compartment, which, along with support cells, form the cellular niche. Such cellular niches within the ECM scaffold together with the surrounding microenvironment contribute important biochemical and physical signals, including growth factors and transcription factors required to initiate stem cell differentiation into committed precursors cells and subsequent precursor cell maturation to form adult tissue cells with specialized phenotypic and functional characteristics.

### Lung Mesenchymal Stem/Progenitor Cells

Tracheal and distal embryonic lung mesenchyme have been demonstrated to have inductive properties for the regional specification of the embryonic epithelium (Shannon & Deterding. Epithelial-mesenchymal interactions in lung development. In: McDonald J A, ed. Lung Biology in Health and Disease. Vol. 100. New York: Marcel Dekker Inc., 1997, pp. 81-118). During lung development, mesenchymal stromal cells at the distal tip of the branching epithelium are known to secrete fibroblast growth factor (FGF)-10, which influences the fate and specificity of early lung epithelial progenitor cells (Bellusci et al. Development. 1997 December; 124(23): 4867-78; Ramasamy et al. Dev Biol. 2007 Jul. 15; 307(2): 237-47). FGF-10 is a component of a multifaceted epithelial-mesenchymal cell signaling network involving BMP, Wnt, and Shh pathways which coordinate the proliferation and differentiation of progenitor cells in the developing lung (reviewed in Morrisey & Hogan. Dev Cell. 2010 Jan. 19; 18(1): 8-23). Lineage tracing studies have also revealed that FGF-10+ mesenchymal cells residing at the branching tip of the epithelium function as stem/progenitor cells for smooth muscle cells, which become distributed along the elongating airways (De Langhe et al., Dev Biol. 2006 Nov. 1; 299(1): 52-62; Mailleuix et al., Development. 2005 May; 132(9): 2157-66). In other studies, mesenchymal stromal cells adjacent to the trachea and extrapulmonary bronchi have also been shown to give rise to bronchiolar smooth muscle cells (Shan et al., Dev Dyn. 2008; 237: 750-5). Collectively, these studies suggest that at least two distinct populations of mesenchymal stromal cells endowed with epithelial modulating properties emerge during development.

Several studies have identified resident mesenchymal stromal cells in adult lungs with the capacity for adipogenic, chondrogenic, osteogenic, and myogenic differentiation. These cells have been clonally expanded from heterogeneous populations of mixed lineage cells defined by their ability to efflux Hoechst 33342 (Giangreco et al., Am J Physiol Lung Cell Mol Physiol. 2004; 286: L624-30; Summer et al., Am J Respir Cell Mol Biol. 2007; 37: 152-9), by their capacity for outgrowth from lung explant cultures (Hoffman et al., Stem Cells Dev. (2011); 20: 1779-92), or by their characteristic expression of Sca-1 (McQualter et al., Stem Cells. (2009); 27: 612-22; Hegab et al, Stem Cells Dev. 2010; 19: 523-36). In addition, further enrichment of CD45-CD31-Sca-1+ mesenchymal stromal cells has been achieved based on their lack of EpCAM expression, which selectively labels epithelial lineage cells (McQualter et al. Proc Natl Acad Sci USA 2010; 107: 1414-19). Resolution of the mesenchymal and epithelial lineages has revealed that the endogenous lung mesenchymal stromal cell population is necessary and sufficient to support the proliferation and differentiation of bronchiolar epithelial stem/progenitor cells in coculture (Id.). This suggests that adult mesenchymal stromal cells share similar epithelial inductive properties to their embryonic counterparts and are an important element of the epithelial stem/progenitor cell niche in the adult lung. This concept is also supported by in vivo studies showing that following naphthalene injury of club cells, parabronchial mesenchymal cells secrete FGF-10 to support epithelial regeneration from surviving epithelial stem/progenitor cells (Volckaert et al., J Clin Invest. 2011; 121: 4409-19).

### Lung Endothelial Progenitor Cells

Endothelial-epithelial cell interactions and angiogenic and angiocrine factors elaborated in the lung epithelial stem/progenitor cell microenvironment also play a role in the regulation of endogenous lung epithelial stem/progenitor cell regeneration and repair (Yamamoto et al., Dev Biol. 2007 Aug. 1; 308(1): 44-53; Ding et al., Cell. 2011 Oct. 28; 147(3): 539-53; Crivellato. Int J Dev Biol. 2011; 55(4-5): 365-75; Grinnell & Harrington. Pulmonary endothelial cell interactions with the extracellular matrix. In: Voelkel N F, Rounds S, eds. The Pulmonary Endothelium: Function in Health and Disease. Chichester, West Sussex: Wiley-Blackwell, 2009: 51-72). For example, it has been reported that the coculture of human vascular endothelial cells with a human bronchial epithelial cell line promotes the generation of branching bronchioalveolar epithelial structures in a 3D culture system (Frazdottir et al. Respir Res. 2010; 11: 162). While considerable progress has been made in understanding the heterogeneity, functional diversity, and pathophysiological behavior of lung vascular and microvascular endothelial cells, the immunophenotypic profiling, quantitation, and functional analysis of lung endothelial progenitor cells (EPC) lags far behind. As for EPC derived from human umbilical cord blood, bone marrow, and mobilized peripheral blood (Timmermans et al., J Cell Mol Med. 2009; 13: 87-102), the rarity of EPC in the lung, their lack of distinguishing markers, and the inability to discriminate circulating EPC and tissue resident EPC have been major impediments in assessing the contribution of endogenous lung EPC in lung vascular repair, and lung regeneration and remodeling (Thebaud & Yoder. Pulmonary endothelial progenitor cells. In: Voelkel N F, Rounds S, eds. The Pulmonary Endothelium: Function in Health and Disease. Chichester, West Sussex: Wiley, 2009: 203-16; Yoder. Proc Am Thorac Soc. 2011; 8: 466-70).

Lung macrovascular and microvascular endothelial cells can be resolved on the basis of their preferential binding to the lectins Helix pomatia and Griffonia simplicifolica, respectively (King et al., Microvasc Res. 2004; 67: 139-51), but there are no other cell surface markers that can discriminate mature lung endothelial cells and EPC (Yoder. Proc Am Thorac Soc. 2011; 8: 466-70). In addition, the rarity of EPC has necessitated the ex vivo expansion and passaging of adherent heterogeneous rat (Alvarez et al., Am J Physiol Lung Cell Mol Physiol. 2008; 294: L419-30) or mouse (Schniedermann et al., BMC Cell Biol. 2010; 11: 50) lung endothelial cells in liquid culture prior to quantitation and flow cytometric and functional analysis of lung-derived EPC in in vitro assays. These assays suggest that the lung microvasculature is a rich source of EPC. However, the incidence, immunophenotypic and functional properties of EPC in the primary explanted endothelial cells compared with their ex vivo manipulated, selected, and expanded counterparts remains indeterminate. The ability of these endogenous lung EPCs to contribute to vascular repair and remodeling in vivo is also unproven (Yoder. Proc Am Thorac Soc. 2011; 8: 466-70). Studies suggest it likely that both circulating EPC and resident lung EPC contribute to endothelial cell regeneration and repair (Balasubramian et al., Am J Physiol Lung Cell Mol Physiol. 2010; 298: L315-23; Duong et al., Angiogenesis. 2011: 411-22; Chamoto et al. Am J Respir Cell Mol Biol. 2012 March; 46(3): 283-9).

Exosomes are released by a wide range of cell types present within the lung including endothelial cells, stem cells, epithelial cells, alveolar macrophage, and tumor cells, although epithelial cells are reported to be the main source of lung-derived exosomes [Alipoor, S D et al. Mediators Inflamm. (2016) 2016: 5628404, citing Y. Fujita, N. Kosaka, J. Araya, K. Kuwano, and T. Ochiya, “Extracellular vesicles in lung microenvironment and pathogenesis,” Trends in Molecular Medicine, vol. 21, no. 9, pp. 533-542, 2015]. Exosomes released by airway epithelial cells contain mucins and alpha 2,6-linked sialic acid which have a neutralizing effect on human influenza virus infection [Id., citing N. T. Eissa, “The exosome in lung diseases: message in a bottle,” Journal of Allergy and Clinical Immunology, vol. 131, no. 3, pp. 904-905, 2013]. Membrane-tethered mucins within epithelial cell-derived exosomes affect the structural properties, conformation, and surface charge of exosomes. The properties of exosomes contribute to mucociliary defense by the lung's innate immune system [Id., citing M. C. Rose and J. A. Voynow, “Respiratory tract mucin genes and mucin glycoproteins in health and disease,” Physiological Reviews, vol. 86, no. 1, pp. 245-278, 2006. Bourdonnay, Z. Zaslona, L. R. K. Penke et al., “Transcellular delivery of vesicular SOCS proteins from macrophages to epithelial cells blunts inflammatory signaling,” The Journal of Experimental Medicine, vol. 212, no. 5, pp. 729-742, 2015].

Repair of lung tissue after viral infection or mechanical trauma

The repair of lung tissue after infection or mechanical trauma normally occurs in a controlled series of events beginning with damage signals sent from infected cells, which recruit inflammatory cells, which then induce secretion of growth factors, which activates basement membrane repair and finally leads to the replacement of injured tissue. Under normal circumstances, the wound healing response is downregulated once the injury is repaired. However, when either the infectious burden overwhelms the system (e.g., with SARS-CoV) or there is persistent damage (e.g., with hepatitis C virus infection), the wound healing response can become dysregulated, resulting in scarring and fibrosis. When fibrosis occurs, it leads to reduced lung function, resulting in a low quality of life and often death. There are limited treatment options for pulmonary fibrosis. Traditionally, corticosteroids are used for the treatment of acute respiratory distress syndrome (ARDS) and pulmonary fibrosis. However, during a viral infection, these interventions dampen the immune response and often result in worsened disease. Due to this lack of therapeutic options, there is a critical need to understand the molecular pathways involved in the development of fibrosis, thus helping to identify novel targets for therapy.

### Pulmonary Fibrosis

Pulmonary fibrosis (PF) occurs in association with a wide range of diseases, including scleroderma (systemic sclerosis), sarcoidosis, and infection, and as a result of environmental exposures (e.g., silica dust or asbestos), but in most patients it is idiopathic and progressive. Pulmonary fibrosis is characterized by parenchymal honeycombing (meaning the characteristic appearance of variably sized cysts in a background of densely scarred lung tissue. Microscopically, enlarged airspaces surrounded by fibrosis with hyperplastic or bronchiolar type epithelium are present. (From https://emedicine.medscape.com/article/2078590-overview), reduced lung compliance, and restrictive lung function (meaning a decreased lung capacity or volume, so a person's breathing rate often increases to meet the oxygen needs on inhalation). Fibrosis of the interstitial spaces (meaning the walls of the air sacs of the lungs (alveoli) and the spaces around blood vessels and small airways) hinders gas exchange, culminating in abnormal oxygenation and clinical dyspnea (meaning shortness of breath, inability to take a deep breath, or chest tightness). Progressive pulmonary fibrosis also leads to pulmonary hypertension, right-sided heart failure, and ultimately respiratory failure (Id.).

Alterations in the WNT signaling pathways are known to contribute to cellular (dys) functions in pulmonary fibrosis (Martin-Medina A, et al. Am J Respir Crit Care Med. (2018) July 25, citing Konigshoff M, et al. PLoS One 2008; 3: e2142; Chilosi M, et al. Am J Pathol (2003); 162: 1495-1502; Selman M, et al. PLoS medicine (2008) 5: e62) and it has been demonstrated that secreted WNT proteins can be transported by EVs to exert their intercellular communication (Id., citing Gross J C, et al. Nat Cell Biol (2012) 14: 1036-1045). The vast majority of research has focused on the role of the WNT/0-catenin pathway in pulmonary fibrosis, which has been linked to disturbed lung epithelial cell function and impaired repair (Id., citing Konigshoff M, et al. PLoS One (2008) 3: e2142; Chilosi M, et al. Am J Pathol (2003) 162: 1495-1502; Selman M, et al. PLoS medicine (2008) 5: e62; Baarsma H A, Konigshoff M. Thorax (2017); 72: 746-759). β-catenin independent WNT signaling in lung fibrosis is much less studied. The WNT protein WNT-5A is largely known to exert its effects 0-catenin independent and has been found upregulated in IPF fibroblasts (Id., citing Vuga L J, et al. Am J Respir Cell Mol Biol. (2009); 41(5): 583-9).

One study showed that lung fibroblasts are a source of EVs and demonstrate autocrine effects of EVs on fibroblast proliferation, which was enhanced by TGF-β (Id.). Similarly, MSC-derived exosomes were found to induce dermal fibroblast proliferation (Id., citing McBride J D, et al. Stem Cells Dev. (2017) 26(19):1384-1398). Fibroblast-derived EVs did not promote myofibroblast differentiation, but rather decreased mRNA levels of myofibroblast markers. MSC-EVs have also been reported to suppress myofibroblast differentiation (Id., citing Fang S, et al. Stem Cells Transl Med. (2016); 5(10): 1425-1439). The proliferative effect of EVs on fibroblasts was to a large extent mediated by WNT-5A, as it was demonstrated that this effect could not only be attenuated by siRNA-mediated WNT-5A knockdown, but also by antibody-mediated neutralization of WNT-5A on EVs or upon destruction of EV structure(Id.). WNT transport on EVs has important implications with respect to the signaling range of WNT proteins, which is generally thought to be rather short and limited to close neighboring cells. EV-mediated transport can contribute to a larger signaling range of WNT proteins and thus determine the signaling outcome on other cells. WNT-5A has also been reported to promote processes as fibroblast adhesion (Id., citing Kawasaki A, et al. Cell Signal. (2007); 19(12): 2498-506) or invasion (Id., citing Waster P, et al. Int J Oncol. (2011); 39(1): 193-202), as well as epithelial-mesenchymal transition (Id., citing Gujral T S, et al. Cell. (2014); 159(4): 844-56). WNT-5A bound EVs in IPF bronchoalveolar lavage fluid (BALF) were shown to contribute to the functional effects, thus suggesting that fibroblast derived EVs can be found in IPF BALF. This work further raises the more general question whether EVs promote lung fibrosis development or might have a protective role in vivo (Id.).

### Idiopathic Pulmonary Fibrosis (IPF)

Idiopathic Pulmonary fibrosis (IPF, also known as cryptogenic fibrosing alveolitis, CFA, or Idiopathic Fibrosing Interstitial Pneumonia) is defined as a specific form of chronic, progressive fibrosing interstitial pneumonia of uncertain etiology that occurs primarily in older adults, is limited to the lungs, and is associated with the radiologic and histological pattern of usual interstitial pneumonia (UIP) (Raghu G. et al., Am J Respir Crit Care Med. (2011) 183(6): 788-824; Thannickal, V. et al., Proc Am Thorac Soc. (2006) 3(4): 350-356). It may be characterized by abnormal and excessive deposition of fibrotic tissue in the pulmonary interstitium. On high-resolution computed tomography (HRCT) images, UIP is characterized by the presence of reticular opacities often associated with traction bronchiectasis. As IPF progresses, honeycombing becomes more prominent (Neininger A. et al., J Biol. Chem. (2002) 277(5): 3065-8). Pulmonary function tests often reveal restrictive impairment and reduced diffusing capacity for carbon monoxide (Thomas, T. et al., J Neurochem. (2008) 105(5): 2039-52). Studies have reported significant increases in TNF-α and IL-6 release in patients with idiopathic pulmonary fibrosis (IPF) (Zhang, Y, et al. J. Immunol. (1993) 150(9): 4188-4196), which has been attributed to the level of expression of IL-1β (Kolb, M., et al. J. Clin. Invest, (2001) 107(12): 1529-1536). The onset of IPF symptoms, shortness of breath and cough, are usually insidious but gradually progress, with death occurring in 70% of patients within five years after diagnosis. This grim prognosis is similar to numbers of annual deaths attributable to breast cancer (Raghu G. et al., Am J Respir Crit Care Med. (2011) 183(6): 788-824).

IPF afflicts nearly 130,000 patients in the United States, with approximately 50,000 new patients annually and nearly 40,000 deaths each year worldwide (Raghu G. et al., Am J Respir Crit Care Med. (2011) 183(6): 788-824). While these data are notable, a recent study reported that IPF may be 5-10 times more prevalent than previously thought, perhaps due to increasing prevalence or enhanced diagnostic capabilities (Thannickal, V. et al., Proc Am Thorac Soc. (2006) 3(4): 350-356). Lung transplantation is considered a definitive therapy for IPF, but the five year survival post lung transplantation is less than 50%. Accordingly, even lung transplantation cannot be considered a “cure” for IPF. In addition to the physical and emotional toll on the patient, IPF is extremely expensive to treat and care for, with national healthcare costs in the range of $2.8 billion dollars for every 100,000 patients annually.

Previous studies have suggested that superimposed environmental insults may be important in the pathogenesis of IPF. In most reported case series, up to 75 percent of index patients with IPF are current or former smokers. In large epidemiologic studies, cigarette smoking has been strongly associated with IPF. In addition, many of the inflammatory features of IPF are more strongly linked to smoking status than to the underlying lung disease. Thus, cigarette smoking may be an independent risk factor for IPF. Latent viral infections, especially those of the herpes virus family, have also been reported to be associated with IPF.

Histopathologically, IPF can be described as accumulation of activated myofibroblasts (or mesenchymal cells) in fibroblastic foci (Thannickal, V. et al., Proc Am Thorac Soc. (2006) 3(4): 350-356). Impaired apoptosis of myofibroblasts may result in a persistent and dysregulated repair process that culminates in tissue fibrosis. Arguably, inflammation also plays a critical role in IPF, perhaps through cyclic acute stimulation of fibroblasts. These findings point to potential targets for therapeutic intervention.

### Pathogenesis of Idiopathic Pulmonary Fibrosis (IPF)

While pathogenic mechanisms are incompletely understood, the currently accepted paradigm proposes that injury to the alveolar epithelium is followed by a burst of pro-inflammatory and fibroproliferative mediators that invoke responses associated with normal tissue repair. For unclear reasons, these repair processes never resolve and progressive fibrosis ensues (Selman M, et al., Ann Intern Med (2001) 134(2): 136-151; Noble, P. and Homer R., Clin Chest Med, 25(4): 749-58, 2004; Strieter, R., Chest (2005) 128 (5 Suppl 1): 526S-532S).

Aside from lung transplantation, potential IPF treatments have included corticosteroids, azathioprine, cyclophosphamide, anticoagulants, and N-acetylcysteine (Raghu G. et al., Am J Respir Crit Care Med. (2011) 183(6): 788-824). In addition, supportive therapies such as oxygen therapy and pulmonary rehabilitation are employed routinely. However, none of these have definitely impacted the long term survival of IPF patients, which further highlights the unmet medical need for treatment options in IPF. As an example, despite mixed clinical program results, InterMune's oral small-molecule Esbriet® (pirfenidone) received European and Japanese approvals for patients with IPF. Esbriet® thus became the first medication specifically indicated for the treatment of IPF; due to equivocal trial outcomes and drug side effects, the drug's utility is viewed with skepticism in the United States, and did not receive an FDA approval based on the data submitted at that time. A large, double-blind, placebo-controlled phase 3 clinical trial to assess the safety and efficacy of pirfenidone in patients with IPF was completed in 2017.

To the best of our knowledge there are no published studies using exosomes from urine of individuals with IPF (U-IPFexo) or any urine-derived exosomes in the investigation of fibrotic lung disease. In the present study we isolated and characterized exosomes isolated from the urine of 16 male individuals with IPF and 10 age and sex-matched control individuals to test the hypothesis that U-IPFexo cargo could promote a fibrotic phenotype. We found a similar miRNA expression pattern in serum and urine derved from the same individual with IPF. Our results also confirmed that several miRNAs previously reported in the lung, serum, and sputum from individuals with IPF could be identified in urine, lung, and serum IPFexo.

Intravenous infusion of urine exosomes isolated from both normal and diseased individuals using in vivo bioluminescent imaging system, revealed fairly rapid biodistribution to the lung. Ex vivo human and mouse lung punch studies and in vivo mouse models were used to functionally assess the effect of tissue (lungs) and systemic (urine and serum) exosomes isolated from individuals with IPF and “control” exosomes isolated from urine and fibroblasts from control lungs. U-IPFexo conveyed a pro-fibrotic lung phenotype and inhibited skin tissue repair. The unique signature was specific to IPFexo, and not only transferred a pro-fibrotic lung phenotype to control mice but further increased markers of lung fibrosis in vivo. Since body weight is independently associated with survival of individuals with IPF, we used body weight as a surrogate for stage of disease and found that mice lost more body weight after treatment with U-IPFexo than with Bleo treatment alone. We also included urine exosomes from subjects with non-cystic fibrosis (non-CF) bronchiectasis or asthma (non-fibrotic lung diseases) in in vivo experiments. Mice receiving these exosomes did not exhibit changes in fibrotic markers. These findings suggest that markers of lung fibrosis are specific, may be detected systemically, outside of the lung, and can communicate the pathology to different tissues. Such body fluid exosomes thus have potential as disease-defining biomarkers in patients with progressive chronic lung diseases, such as IPF.

### SUMMARY OF THE INVENTION

According to one aspect, the present disclosure provides a method for noninvasively diagnosing and staging a progressive chronic lung disease characterized by disease related lung dysfunction comprising: (a) quantifying lung function of a subject with a progressive chronic lung disease characterized by disease-related lung dysfunction; (b) diagnosing and determining a stage of the chronic lung disease by: (i) collecting a biological sample from the subject with the lung disease and from a healthy control, (ii) centrifuging the biological sample at low speed to form a clarified supernatant; (iii) ultra centrifuging the clarified supernatant to pellet the purified enriched population of exosomes; and (iv) characterizing the purified enriched population of exosomes, wherein. (1) the population of exosomes expresses two or more exosome biomarkers selected from the group consisting of CD9, CD63, CD81, or HSP70; (2) size of the exosomes in the population of exosomes ranges from 30 μm to 150 μm, inclusive; (3) the population of exosomes comprises a total protein of at least 100-200 μg, inclusive; (4) the population of exosomes comprise a total RNA content of at least 100-200 ng, inclusive; (5) number of exosomes in the population of exosomes comprises at least 10E10 particles; and (6); the population of exosomes comprises a cargo comprising dysregulated expression of two or more microRNAs selected from miR-199, miR Let-7a, miR Let-7b, mir-Let-7d, miR-10a, miR-21, miR-29a, miR-34, miR-101, miR-125, miR-145, miR-146a, miR-181a, miR-181b, miR-181c, miR-199, and miR-142, wherein expression of the two or more microRNAs, compared to a healthy control, comprises a signature of a fibrotic lung disease; and (c) medically managing the diagnosed fibrotic lung disease as early as possible in the course of progression of the disease to reduce or slow its progression by (1) treating the diagnosed fibrotic lung disease; and (2) monitoring over time the population of exosomes derived from the biological sample of the subject compared to a population of exosomes derived from a biological sample derived from a healthy control.

According to some embodiments of the method, the lung function of the subject is quantified by determining forced expiratory volume (FEV1); forced vital capacity (FVC) and FEV/FVC %. According to some embodiments the biological sample is a body fluid and the body fluid is amniotic fluid, blood, breast milk, saliva, urine, bile, pancreatic juice, cerebrospinal fluid or a peritoneal fluid. According to some embodiments the body fluid is serum or urine. According to some embodiments the body fluid is serum. According to some embodiments the body fluid is urine. According to some embodiments the biological sample is obtained from a mammal. According to some embodiments the biological sample is obtained from a non-human mammal or a human subject. According to some embodiments, the human subject is over 50 years of age. According to some embodiments the healthy control is age and sex matched to the subject.

According to some embodiments the chronic lung disease if left untreated comprises one or more of a progressive injury, progressive inflammation, progressive fibrosis or a combination thereof. According to some embodiments the fibrotic lung disease is interstitial pulmonary fibrosis (IPF).

According to some embodiments, dysregulated miRNA expression of miR-199 and miR-34a in the population of exosomes derived from the biological sample of the subject compared to a population of exosomes derived from a biological sample derived from a healthy control is fibrotic.

According to some embodiments dysregulated miRNA expression of miR-142 in the population of exosomes derived from the biological sample of the subject compared to a population of exosomes derived from a biological sample derived from a healthy control is antifibrotic.

According to some embodiments the dysregulated expression of one or more of miR let-7D, miR-29, or miR-181 in the population of exosomes derived from the biological sample of the subject compared to a population of exosomes derived from a biological sample derived from a healthy control is decreased, compared to the population of exosomes derived from the healthy control.

According to some embodiments, dysregulated expression of miR-199 in the population of exosomes derived from the biological sample of the subject compared to a population of exosomes derived from a biological sample derived from a healthy control is increased and the expression of one or more of miR-let-7d, miR-29, miR-142, miR-181 in the population of exosomes derived from the biological sample of the subject compared to a population of exosomes derived from a biological sample derived from a healthy control is decreased.

According to some embodiments reduced or absent mR Let-7 expression in the population of exosomes derived from the biological sample of the subject compared to a population of exosomes derived from a biological sample derived from a healthy control compared to a healthy control and is associated with early stage fibrotic lung disease.

According to some embodiments; micro RNA expression in the population of exosomes derived from the biological sample of the subject compared to a population of exosomes derived from a biological sample derived from a healthy control comprises increased miR-Let-7 expression compared to a healthy control and is associated with end-stage fibrotic lung disease.

According to some embodiments the medical managing modulates one or more of an injury, inflammation, an excess accumulation of extracellular matrix, cell senescence; or a pathway comprising fibrogenic signaling. According to some embodiments markers for fibrosis and downstream fibrotic pathways comprise αᵥ-integrin, collagen type I mRNA expression, c-Jun, AKT expression and MMP-9 activity. According to some embodiments the pathway comprising fibrogenic signaling is one or more of a Smad pathway, a mitogen-activated protein kinase pathway, a phosphoinositide 3-kinase pathway; a canonical Wnt-β catenin pathway, and a Notch signaling pathway. According to some embodiments the pathway comprising fibrogenic signaling comprises transforming growth factor (TGFβ) signaling.

According to some embodiments the treating of the fibrotic disease includes administering an active therapeutic agent including an immunomodulator, an analgesic, an anti-inflammatory agent, an anti-fibrotic agent, an anti-viral agent, a proton pump inhibitor, or oxygen therapy. According to some embodiments the immunomodulator includes prednisone, azathioprine, mycophenolate, mycophenolate mofetil, colchicine, or interferon-gamma 1b. According to some embodiments the analgesic includes capsaisin, codeine, hydrocodone, lidocaine, oxycodone, methadone, resiniferatoxin, hydromorphone, morphine, and fentanyl. According to some embodiments the anti-inflammatory agent includes aspirin, celecoxib, diclofenac, diflunisal, etodolac, ibuprofen, indomethacin, ketoprofen, ketorolac nabumetone, naproxen, nintedanib, oxaprozin, pirfenidone, piroxicam, salsalate, sulindac, and tolmetin. According to some embodiments the anti-fibrotic agent includes nintedanib or pirfenidone. According to some embodiments the anti-viral agent includes acyclovir, gancidovir, foscarnet; ribavirin; amantadine, azidodeoxythymidine/zidovudine), nevirapine, a tetrahydroimidazobenzodiazepinone (TIBO) compound; efavirenz; remdecivir, or delavirdine. According to some embodiments the proton pump inhibitor includes omeprazole, lansoprazole, dexlansoprazole, esomeprazole, pantoprazole, rabeprazole, or ilaprazole.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A. Transmission electron microscopy of isolated exosomes. Image magnification Scale bar=50 nm. FIG. 1B shows that isolated exosomes express CD63.

FIG. 2A, FIG. 2B, FIG. 2C and FIG. 2D shows that expression of miR let-7d (A), miR-29a-5p (B), miR 181b-3p (C) and miR-199a-3p (D) in urine-derived exosomes reveals a pattern corresponding to that reported in serum and whole lung of individuals with IPF. PCR was performed on extracted urine-derived exosomes as described in methods. Data are graphed as relative miRNA expression normalized to U6 and percent of control expression. * P<0.05, **P<0.01 compared to control exosomes. Each point represents an individual patient exosome sample. N=5-12 individual samples/group, P values were calculated by Mann-Whitne U test.

FIG. 2E shows that Urine and serum-derived exosomes isolated from the same individuals with IPF have similar miRNA expression. Exosome isolation, RNA preparation and PCR performed as described in methods. n=5 individual samples of urine and serum-derived exosomes. Paired T test analysis was performed.

FIG. 3A, FIG. 3B and FIG. 3C illustrate the biodistribution of circulating urine-derived exosomes. Shown are representative in vivo bioluminescence images to study the biodistribution of ExoGlow™ labeled urine-derived exosomes in mice (n=3/group) at the indicated time points. FIG. 3A is a panel showing a mouse injected with labeled U-IPF exo; FIG. 3B is a panel showing a mouse injected with labeled urine-derived exosomes from age and sex-matched control individuals without lung disease. FIG. 3C is a panel showing a mouse injected with PBS. Intensity of luminescence seen in bar from lowest (red) to highest (blue). n=3 individual exosome preparations/group.

FIG. 4A, FIG. 4B, FIG. 4C, FIG. 4D, FIG. 4E, FIG. 4F, and FIG. 4G show representative TEM photos of lung punches. FIG. 4A, FIG. 4B, and FIG. 4C show mouse lung punches injected with gold nanoparticle labeled urine-derived exosomes from age and sex-matched control subjects (without lung disease) or U-IPFexo (FIG. 4D, FIG. 4E, FIG. 4F, FIG. 4G, FIG. 411). TEM revealed exosomes in alveolar epithelial cells (AEC) type I and type II. Arrows in FIG. 4C, FIG. 4F, and FIG. 4G highlight exosomes containing nanoparticles. n=2 individual exosome preparations/group.

FIG. 5A FIG. 5B, FIG. 5C, FIG. 5D, FIG. 5E, and FIG. 5F show immunofluorescence staining of lung punches injected with exosomes derived from urine (FIG. 5B, FIG. 5C) or myofibroblasts or fibroblasts (FIG. 5E, FIG. 5F). Lung punches were fixed four days post injection with either PBS (FIG. 5A or FIG. 5D) or control urine-derived exosomes (FIG. 5B), U-IPFexo (FIG. 5C), control fibroblast (FIG. 5E) or MF-IPF exosomes (FIG. 5F). Shown are representative merged photographs at 20×, surfactant protein C (SPC, red), αSMC actin (green) and DAPI (blue). n=3 individual exosome preparations/group.

FIG. 6A, FIG. 6B, FIG. 6C, FIG. 6D, FIG. 6E, FIG. 6F, FIG. 6G, FIG. 6H, FIG. 6I, FIG. 6J, FIG. 6K, and FIG. 6L show that fibrotic pathways [see FIG. 12] are activated in lung punches after injection with urine (U-IPFexo) or myofibroblast-derived (MF-IPFexo) exosomes. Human (FIG. 6A, FIG. 6B, FIG. 6C) and mouse lung (FIG. 6D, FIG. 6E, FIG. 6F, FIG. 6G, FIG. 6H, FIG. 6I, FIG. 6J, FIG. 6K, FIG. 6L, FIG. 6M) punches were injected with PBS alone, U-IPFexo [FIG. 6D, FIG. 6E, FIG. 6F, FIG. 6G, FIG. 6H, FIG. 6I] or MF-IPFexo FIG. 6J, FIG. 6K, FIG. 6L, FIG. 6M or age and sex-matched control urine exosomes or lung fibroblast exosomes from control subjects (without lung disease). Punches were collected 4 days later and processed as described in Methods. Human lung punches were injected with MF-IPFexo or fibroblast cell derived exosomes (FIG. 6A and FIG. 6B) or urine-derived control and U-IPFexo (FIG. 6B and FIG. 6C). n=2 human lung punch isolates, 2 individual exosome preparations/group. Data are graphed as percent PBS control. αᵥ-integrin (FIG. 6A, FIG. 6D and FIG. 6J) and collagen type 1 (FIG. 6A, FIG. 6E and FIG. 6K) mRNA expression increased in punches injected with U-IPFexo (derived from urine or myofibroblasts). Data are graphed as normalized for 18S content, n=3-7 individual exosome preparations/group. * P<0.05 compared to PBS, **P<0.01 compared to control exosomes. Downstream fibrotic pathways (see FIG. 12); ERα (FIG. 6C, FIG. 6F, and FIG. 6L), activated AKT (FIG. 6H), c-Jun (FIG. 6 G and FIG. 6M), protein expression and MMP-9 activity (FIG. 6 I) were also stimulated by exosomes from individuals with IPF. * P<0.05, **P<0.01 compared to PBS or control exosomes. P values were calculated by Mann Whitney U test.

FIG. 7A and FIG. 7B show that epithelization in ex vivo wound healing is decreased by urine-derived IPF exosomes (U-IPFexo). Human skin was wounded, injected with U-IPFexo or control (age and sex-matched from individuals without lung disease) exosomes and maintained at the air-liquid interface. Wound healing was assessed at day 4 post-wounding, a time point when exponential epithelialization occurs. FIG. 7A Data are graphed as mean with each data point representing a single wound. Experiments were performed using triplicate technical replicates and 2-3 biological replicates. P<0.005 PBS and control compared to IPF, PBS vs. Control=0.05 P values were calculated by Mann Whitney U test. FIG. 7B, show photos of gross skin showing visual signs of closure and correspond to the histology assessments. Black arrows point to the initial site of wounding, while white arrows point to the wound edge of the migrating epithelial tongue. Scale bars, 500 μm proportional to the image size.

FIG. 8A, FIG. 8B, FIG. 8C, FIG. 8D, FIG. 8E, FIG. 8F, FIG. 8G, FIG. 8H, FIG. 8I, FIG. 8J, FIG. 8K, FIG. 8L, FIG. 8M, FIG. 8N, and FIG. 8O illustrate an assessment of fibrosis in Bleomycin (Bleo) treated mice intravenously infused with exosomes derived from the urine of individuals with IPF (U-IPFexo) compared to infusion with urine exosomes derived from age and sex-matched control subjects without lung disease or urine exosomes derived from subjects with non-CF bronchiectasis or asthma (non-fibrotic lung disease). Histological sections of lung tissue were stained with Masson's-Trichrome as described in Materials and Methods. Representative photomicrographs (4×, 10×, and 20×) of lung sections from Bleo+vehicle (FIG. 8A, FIG. 8B, FIG. 8C), Bleo+control exosome injected mice (FIG. 8D, FIG. 8E, FIG. 8F), from Bleo+U-IPFexo injected mice (FIG. 8G, FIG. 8H, FIG. 8I) or from non-fibrotic inducing exosomes (Bronchiectasis, FIG. 8J, FIG. 8K, FIG. 8L). Fibrotic score (FIG. 8M), collagen content (FIG. 8N), avintegrin (FIG. 8O) increased after Bleo+U-IPFexo treatment. FIG. 8M shows Ashcroft scores were used to evaluate the degree of fibrosis. Data are graphed as the mean score of 32 fields/section of lung. FIG. 8N shows collagen content was estimated by hydroxyproline assay as described in Methods. Data are graphed as g/mg of lung tissue. FIG. 8O shows αv-integrin mRNA expression, which was determined by RT-PCR as a marker of fibrosis. Data are graphed normalized for 18S content. Each data point represents an individual mouse, n=3-11/group *p<0.05, ** p<0.001, p<0.0001 compared to control exosome treatment or compared to Bleo+vehicle treatment. Data were analyzed using one-way analysis of variance (ANOVA) and Mann-Whitney U test.

FIG. 9A and FIG. 9B show lung fluorescence intensity over time of mice injected with urine-derived exosomes from individuals with IPF (U-IPFexo), urine-derived exosomes from individuals without IPF (Control exosomes), and PBS. FIG. 9C shows ex-vivo fluorescence imaging of isolated organs at 48 hours following exosome treatment in mice.

FIG. 10A, FIG. 10B, FIG. 10C and FIG. 10D show histology and trichrome staining of lung punches from C57BL6 mice. Lung punches from control lungs shown in tissue culture dish (FIG. 10A) have normal histology (FIG. 10B, Trichrome staining 10×mag) and structure by TEM (FIG. 10C, 500 mag). Histology of non-injected lung punch (FIG. 10D).

FIG. 11 shows that collagen content increases in mice receiving urine derived exosomes from individuals with IPF. Naïve mice were treated with PBS, control or IPF urine-derived exosomes. Mice were sacrificed 21 days later as described in methods. Data are graphed as mean±SEM. Each data point represents an individual mouse (n=2 exosome preps/group). P<0.05 IPF compared to control and PBS, Data were analyzed using Mann Whitney test.

FIG. 12 shows potential microRNA regulated pathways leading to fibrosis. The genes and biological processes in the network are generated from the IPF vs Control Lung dataset from NCBI GEO (GS21369) of 11 IPF samples and 6 healthy lung samples.

FIG. 13 shows an overview of the experimental details and design.

### DETAILED DESCRIPTION OF THE INVENTION

### Definitions

As used herein, the term “about” means plus or minus 20% of the numerical value of the number with which it is being used. Therefore, about 50% means in the range of 40%-60%.

The term “activation” or “lymphocyte activation” refers to stimulation of lymphocytes by specific antigens, nonspecific mitogens, or allogeneic cells resulting in synthesis of RNA, protein and DNA and production of lymphokines; it is followed by proliferation and differentiation of various effector and memory cells. T-cell activation is dependent on the interaction of the TCR/CD3 complex with its cognate ligand, a peptide bound in the groove of a class I or class II MHC molecule. The molecular events set in motion by receptor engagement are complex. Full responsiveness of a T cell requires, in addition to receptor engagement, an accessory cell-delivered costimulatory activity, e.g., engagement of CD28 on the T cell by CD80 and/or CD86 on the antigen presenting cell (APC).

The term “activator protein 1 (APi) as used herein refers to a heterodimeric transcription factor composed of members of the Jun and Fos family of basic leucine zipper proteins. These include the Jun proteins c-Jun, JunB and JunD, as well as the Fos proteins c-Fos, Fra2 and Fosb, respectively. C-Jun in combination with protein c-Fos forms the AP-1 early response transcription factor.

The term “adaptive immunity” as used herein refers to a specific, delayed and longer-lasting response by various types of cells that create long-term immunological memory against a specific antigen. It can be further subdivided into cellular and humoral branches, the former largely mediated by T cells and the latter by B cells. This arm further encompasses cell lineage members of the adaptive arm that have effector functions in the innate arm, thereby bridging the gap between the innate and adaptive immune response.

The term “adipose stem cell,” “adipose-derived stem cell,” or “ASC” as used herein refers to pluripotent stem cells, mesenchymal stem cells, and more committed adipose progenitors and stroma obtained from adipose tissue.

“Administering” when used in conjunction with a therapeutic means to give or apply a therapeutic directly into or onto a target organ, tissue or cell, or to administer a therapeutic to a subject, whereby the therapeutic positively impacts the organ, tissue, cell, or subject to which it is targeted. Thus, as used herein, the term “administering”, when used in conjunction with EVs or compositions thereof, can include, but is not limited to, providing EVs into or onto the target organ, tissue or cell; or providing EVs systemically to a patient by, e.g., intravenous injection, whereby the therapeutic reaches the target organ, tissue or cell. “Administering” may be accomplished by parenteral, oral or topical administration, by inhalation, or by such methods in combination with other known techniques.

The term “alveolus” or “alveoli” as used herein refers to an anatomical structure that has the form of a hollow cavity. Found in the lung, the pulmonary alveoli are spherical outcroppings of the respiratory sites of gas exchange with the blood. The alveoli contain some collagen and elastic fibers. Elastic fibers allow the alveoli to stretch as they fill with air when breathing in. They then spring back during breathing out, in order to expel the carbon dioxide-rich air.

The term “angiotensin II” or “Ang-2” as used herein refers to a vasoactive octapeptide produced by the action of angiotensin-converting enzyme on angiotensin 1; it produces stimulation of vascular smooth muscle, promotes aldosterone production, and stimulates the sympathetic nervous system.

The terms “animal,” “patient,” and “subject” as used herein include, but are not limited to, humans and non-human vertebrates such as wild, domestic and farm animals. According to some embodiments, the terms “animal,” “patient,” and “subject” may refer to humans. According to some embodiments, the terms “animal,” “patient,” and “subject” may refer to non-human mammals.

The term “antioxidant” as used herein refers to any substance that can prevent, reduce, or repair the ROS-induced damage of a target biomolecule. In ROS biology and medicine, the target molecules usually include proteins, lipids, and nucleic acids, among others. There are three major modes of action for antioxidants: (i) antioxidants that directly scavenge ROS already formed; (ii) antioxidants that inhibit the formation of ROS from their cellular sources; and (iii) antioxidants that remove or repair the damage or modifications caused by ROS. [Li, R., et al. React. Oxyg. Species (Apex) (2016): 1 (1): 9-21].

The term “aquaporins” as used herein refers to water-specific membrane channel proteins. Aquaporin 5 (AQP5) is found in airway epithelial cells, type I alveolar epithelial cells and submucosal gland acinar cells in the lungs where it plays a key role in water transport.[Hansel, N N et al. PLoS One (2010) doi.10.1371/journal.pone.0014226, citing Verkman, A S et al. Am. J. Physiol. Lung Cell Mol. Physiol. 278 (5): L867-79] Decreased expression of human AQP5 has been associated with mucus overproduction in the airways of subjects with COPD and lower lung function.[Id., citing Wang, K. et al. Acta Pharmacol. Sin. (2007) 28 (8): 1166-74] Furthermore, smoking has been shown to attenuate the expression of AQP5 in submucosal glands of subjects with COPD.[Id., citing Id, citing Wang, K. et al. Acta Pharmacol. Sin. (2007) 28 (8): 1166-74]

The term “Argonaute 2” or “AGO2” as used herein refers to an RNA binding protein that can shuttle between the cytoplasm and nucleus in a context-dependent fashion [Sharma, N R et al. J. Biol. Chem. (2016) 291: 2302-9] and is a key effector of RNA-silencing pathways. It is a major component of the RNA-induced silencing complex RISC).

The term “Ashcroft scale for the evaluation of bleomycin-induced lung fibrosis” refers to the analysis of stained histological samples by visual assessment. A modified Ashcroft scale precisely defines the assignment of grades from 0 to 8 for the increasing extent of fibrosis in lung histological samples. [Hubner, R-H et al. Biotechniques (2008) 44 (4): 507-11].

The term “biomarkers” (or “biosignatures”) as used herein refers to peptides, proteins, nucleic acids, antibodies, genes, metabolites, or any other substances used as indicators of a biologic state. It is a characteristic that is measured objectively and evaluated as a cellular or molecular indicator of normal biologic processes, pathogenic processes, or pharmacologic responses to a therapeutic intervention. The term “indicator” as used herein refers to any substance, number or ratio derived from a series of observed facts that may reveal relative changes as a function of time; or a signal, sign, mark, note or symptom that is visible or evidence of the existence or presence thereof. Once a proposed biomarker has been validated, it may be used to diagnose disease risk, presence of disease in an individual, or to tailor treatments for the disease in an individual (choices of drug treatment or administration regimes). In evaluating potential drug therapies, a biomarker may be used as a surrogate for a natural endpoint, such as survival or irreversible morbidity. If a treatment alters the biomarker, and that alteration has a direct connection to improved health, the biomarker may serve as a surrogate endpoint for evaluating clinical benefit. Clinical endpoints are variables that can be used to measure how patients feel, function or survive. Surrogate endpoints are biomarkers that are intended to substitute for a clinical endpoint; these biomarkers are demonstrated to predict a clinical endpoint with a confidence level acceptable to regulators and the clinical community.

The term “bleomycin-induced pulmonary fibrosis model” as used herein refers to an animal model of pulmonary fibrosis in rodents. It causes inflammatory and fibrotic reactions within a short period of time, even more so after intratracheal instillation. The initial elevation of pro-inflammatory cytokines (interleukin-1, tumor necrosis factor-α, interleukin-6, interferon-γ) is followed by increased expression of pro-fibrotic markers (transforming growth factor-β1, fibronectin, procollagen-1), with a peak around day 14. [Moeller, A. et al. Int. J. Biochem. Cell Biol. (2008) 40 (3): 362-82]. The “switch” between inflammation and fibrosis appears to occur around day 9 after bleomycin (Id., citing Chaudhary, N I et al. Am. J. Respir. Crit. Care Med. (2006) 173 (7): 769-76). While the bleomycin model has the advantage that it is quite easy to perform, widely accessible and reproducible, and therefore fulfills important criteria expected from a good animal model. the bleomycin model has significant limitations in regard to understanding the progressive nature of human IPF. While bleomycin causes an inflammatory response, triggered by overproduction of free radicals, with induction of pro-inflammatory cytokines and activation of macrophages and neutrophils, thus resembling acute lung injury in some way, the subsequent development of fibrosis, however, is at least partially reversible, independent from any intervention (Izbicki, G et al. Intl J. Exp. Pathol. (2002) 83 (3): 111-19), and the aspect of slow and irreversible progression of IPF in patients is not reproduced in the bleomycin model (Chua, F. et al. Am. J. Respir. Cell Mol. Biol. (2005) 33 (1): 9-13).

The term “bronchiectasis” as used herein refers to abnormal widening of the bronchi or their branches, causing a risk of infection.

The term “bronchoalveolar lavage” (BAL) is used herein to refer to a medical procedure in which a bronchoscope is passed through the mouth or nose into the lungs and fluid is squirted into a small part of the lung and then collected for examination. “Bronchoalveolar lavage fluid” (BALF) is used herein to refer to the fluid collected from a BAL procedure. Bronchoalveolar lavage (BAL), performed during fiberoptic bronchoscopy is a useful adjunct to lung biopsy in the diagnosis of nonneoplastic lung diseases. BAL is able to provide cells and solutes from the lower respiratory tract and may provide important information about diagnosis and yield insights into immunologic, inflammatory, and infectious processes taking place at the alveolar level. BAL has been helpful in elucidating the key immune effector cells driving the inflammatory response in IPF (Costabel and Guzman Curr Opin Pulm Med, 7 (2001), pp. 255-261). Increase in polymorphonuclear leukocytes, neutrophil products, eosinophils, eosinophil products, activated alveolar macrophages, alveolar macrophage products, cytokines, chemokines, growth factors for fibroblasts, and immune complexes have been noted in BAL of patients with IPF. [Id.].

The term “c-jun” as used herein refers to a protein of the activator protein-1 complex, which is involved in numerous cell activities, including proliferation, apoptosis, survival, tumorigenesis and tissue morphogenesis. As a basic leucine zipper (bZIP) transcription factor, it acts as a homo- or heterodimer, binding to DNA and regulating gene transcription. It is able to crosstalk, amplify and integrate different signals for tissue development and disease.[Meng, Q. and Xia, Y. Protein Cell (2011) 2 (11): 889-98].

The term “cargo” as used herein refers to a load or that which is conveyed. With respect to exosomes and or extracellular vesicles, the term cargo refers to a substance encapsulated in the exosome and or extracellular vesicle. The compound or substance can be, e.g., a nucleic acid (e.g., nucleotides, DNA, RNA), a polypeptide, a lipid, a protein, or a metabolite, or any other substance that can be encapsulated in an exosome and or extracellular vesicle.

The term “cargo profile” as used herein refers to measurements of cargo components that characterize a population of extracellular vesicles

The term “caveolins (Cavs)” as used herein refers to integrated plasma membrane proteins that are complex signaling regulators with numerous partners and whose activity is highly dependent on cellular context (Boscher, C, Nabi, I R. Adv. Exp. Med. Biol. (2012) 729: 29-50). Cavs are both positive and negative regulators of cell signaling in and/or out of caveolae, invaginated lipid raft domains whose formation is caveolin expression dependent. Caveolins and rafts have been implicated in membrane compartmentalization; proteins and lipids accumulate in these membrane microdomains where they transmit fast, amplified and specific signaling cascades. The term “caveolin 1 (CAV1)”, refers to a scaffolding protein that links integrin subunits to the tyrosine kinase FYN, an initiating step in coupling integrins to the Ras-ERK pathway and promoting cell cycle progression.

“Cluster of Differentiation” or “cluster of designation” (CD) molecules are utilized in cell sorting using various methods, including flow cytometry. Cell populations usually are defined using a “+” or a “−” symbol to indicate whether a certain cell fraction expresses or lacks a particular CD molecule.

The term “CD9” as used herein refers to a member of the tetraspanin protein family whose crystal structure shows a reversed cone-like molecular shape, which generates membrane curvature in the crystalline lipid layers. (Umeda, R. et al. Nature Communic. (2020) 11: article 1606).

The term “CD29” as used herein refers to integrin R31.

The term “CD37” as used herein refers to a member of the tetraspanin protein family exclusively expressed on immune cells. (Zuidscherwoude, M. et al. Scientific Reports (2015) 5: 12201).

The term “CD44” as used herein refers to a cell adhesion molecule (HCAM) found on monocytes, neutrophils, fibroblasts and memory T cells, which is involved in lymphocyte homing.

The term “CD63” as used herein refers to a member of the tetraspanin protein family, the C-terminal domain of which interacts with several subunits of adaptor protein (AP) complexes, linking the traffic of this tetraspanin to clathrin-dependent pathways (Andreu, Z. & Yanez-Mo, M., citing Rous, B A et al. Mol. Biol. Cell (2002) 13 (3): 1071-82). Among intracellular interacting proteins, CD63 was shown to directly bind to syntenin-1, a double PDZ domain-containing protein (Id., citing Latysheva, N. et al. Mol. Cell Biol. (2006) 26 (20): 7707-18). A major role in exosome biogenesis has been reported for Syntenin-1 (Id., citing Baietti, M F et al. Nat. Cell Biol. (2012) 14 (7): 677-85).

The term “CD81” as used herein refers to a member of the tetraspanin protein family whose crystal structure shows a reversed teepee-like arrangement of the four transmembrane I helices, which create a central pocket in the intramembranous region that appears to bind cholesterol in the central cavity. (Zimmerman, B. et al. Cell (2016) 167: 1041-51). During development, CD81 regulates the trafficking of CD19, an essential co-stimulatory molecule of lymphoid B cells and a well-characterized CD81 partner, along the secretory pathway. (Shoham, T. et al. J. Imunol. (2003) 171: 4062-72). CD9 and CD81 have been shown to regulate several cell-cell fusion processes. (Charrin, S. et al. J. Cell Science (2014) 127: 3641-48).

The term “CD82” as used herein refers to a member of the tetraspanin protein family that has been implicated in the regulation of protein sorting into EVs and in antigen presentation by antigen presenting cells. (Andreu, Z. and Yanez-Mo, M. Front. Immunol. (2014) doi.org/10.3389/fimmu.2014.00442).

The term “CD105” refers to endoglin, a cell membrane glycoprotein and part of the transforming growth factor-β receptor complex, which plays a role in angiogenesis.

The term “cell reprogramming” as used herein refers to a process by which transcription factors inducing cells to revert to an earlier stage of development. It includes reverting mature differentiated cells into immature stem or progenitor cells and then differentiating those stem or progenitor cells; a process of converting somatic cells into other cell types without the need for an intermediate pluripotent state (direct cell reprogramming); or direct conversion of one differentiated cell type into another, also known as transdifferentiation. [Wilmut, I. et al. Philos. Trans. R. Soc. London B. Biol. Sci. (2011) 366 (1575): 2183-97].

The term “cellular senescence” as used herein refers to a process that results from a variety of stresses and leads to a state of irreversible growth arrest. A variety of cell-intrinsic and -extrinsic stresses can activate the cellular senescence program. These stressors engage various cellular signaling cascades but ultimately activate p53, p16Ink4a, or both. Cells exposed to mild damage that can be successfully repaired may resume normal cell-cycle progression. On the other hand, cells exposed to moderate stress that is chronic in nature or that leaves permanent damage may resume proliferation through reliance on stress support pathways (green arrows). This phenomenon (termed assisted cycling) is enabled by p53-mediated activation of p21. Thus, the p53-p21 pathway can either antagonize or synergize with p16Ink4a in senescence depending on the type and level of stress. Cells undergoing senescence induce an inflammatory transcriptome regardless of the senescence inducing stress. [van Deursen, J M. Nature (2014) 509 (7501): 439-46]. Senescent cells accumulate in tissues and organs with age [Id., citing Lawless, C. Exp. Gerontol. (2010) 45: 772-78; Krishnamurthy, J. et al Nature (2006) 443: 453-57; Jeyapalan, J C et al. Mech Ageing Dev. (2007) 128: 36-44] as well as at sites of tissue injury and remodeling [Id., citing Rajagopalan, S and Long, E O. Proc. Natl. Acad. Sci. USA (2012) 109: 20596-601; Munoz-Espin, D. et al. Cell (2013) 155: 1104-18; Storer, M. et al. Cell (2013) 155: 1119-30; Jun, J I and Lau, L F. Nature Cell Biol. (2010) 12: 676-85; Krizhanovsky, V. et al. Cell (2008) 134: 657-67].

The term “Chronic Obstructive Pulmonary Disease” as used herein refers to a lung disease that causes obstructed airflow in the lungs and results in breathing problems. It is used to describe such lung diseases as emphysema, chronic bronchitis, and severe asthma. COPD affects 9-10% of the adult population in the United States; it is the third leading cause of death and the 12^{th} leading cause of morbidity. By 2030, it is expected to be the 4^{th} leading cause of death worldwide, representing over 4.5 million deaths annually.

The term “contact” and its various grammatical forms as used herein refers to a state or condition of touching or of immediate or local proximity.

The term “cytokine” as used herein refers to small soluble protein substances secreted by cells which have a variety of effects on other cells. Cytokines mediate many important physiological functions including growth, development, wound healing, and the immune response. They act by binding to their cell-specific receptors located in the cell membrane, which allows a distinct signal transduction cascade to start in the cell, which eventually will lead to biochemical and phenotypic changes in target cells. Generally, cytokines act locally. They include type I cytokines, which encompass many of the interleukins, as well as several hematopoietic growth factors; type II cytokines, including the interferons and interleukin-10; tumor necrosis factor (“TNF”)-related molecules, including TNFα and lymphotoxin; immunoglobulin super-family members, including interleukin 1 (“IL-1”); and the chemokines, a family of molecules that play a critical role in a wide variety of immune and inflammatory functions. The same cytokine can have different effects on a cell depending on the state of the cell. Cytokines often regulate the expression of, and trigger cascades of, other cytokines.

The terms “D value” or “mass division diameter” as used herein, refer to the diameter which, when all particles in a sample are arranged in order of ascending mass, divides the sample's mass into specified percentages. The percentage mass below the diameter of interest is the number expressed after the “D”. For example, the D10 diameter is the diameter at which 10% of a sample's mass is comprised of smaller particles, and the D50 is the diameter at which 50% of a sample's mass is comprised of smaller particles. The D50 is also known as the “mass median diameter” as it divides the sample equally by mass. The D90 diameter is the diameter at which 90% of a sample's mass is comprised of smaller particles. While D-values are based on a division of the mass of a sample by diameter, the actual mass of the particles or the sample does not need to be known. A relative mass is sufficient as D-values are concerned only with a ratio of masses. This allows optical measurement systems to be used without any need for sample weighing.

From the diameter values obtained for each particle a relative mass can be assigned according to the following relationship:

Mass of a sphere=π/6d³ρ

Assuming that ρ is constant for all particles and cancelling all constants from the equation:

Relative mass=d³

i.e., each particle's diameter is therefore cubed to give its relative mass. These values can be summed to calculate the total relative mass of the sample measured. The values may then be arranged in ascending order and added iteratively until the total reaches 10%, 50% or 90% of the total relative mass of the sample. The corresponding D value for each of these is the diameter of the last particle added to reach the required mass percentage.

As used herein, the term “derived from” is meant to encompass any method for receiving, obtaining, or modifying something from a source of origin.

As used herein, the terms “detecting”, “determining”, and their other grammatical forms, are used to refer to methods performed for the identification or quantification of a biomarker, such as, for example, the presence or level of miRNA, or for the presence or absence of a condition in a biological sample. The amount of biomarker expression or activity detected in the sample can be none or below the level of detection of the assay or method.

The terms “disease” or “disorder” as used herein refer to an impairment of health or a condition of abnormal functioning. The term “fibrotic disease” as used herein refers to a condition marked by an increase of interstitial fibrous tissue. The terms “lung tissue disease” or “lung disease” as used herein refers to a disease that affects the structure of the lung tissue, for example, without limitation, pulmonary interstitium. Scarring or inflammation of lung tissue makes the lungs unable to expand fully (“restrictive lung disease”). It also makes the lungs less capable of taking up oxygen (oxygenation) and releasing carbon dioxide. Examples of lung tissue diseases include, but are not limited to, idiopathic pulmonary fibrosis (IPF), acute lung injury (ALI), radiation-induced fibrosis in the lung, a fibrotic condition associated with lung transplantation, and sarcoidosis, a disease in which swelling (inflammation) occurs in the lymph nodes, lungs, liver, eyes, skin, or other tissues. According to some embodiments, pulmonary fibrosis is due to acute lung injury caused by viral infection, including, without limitation, influenza, SARS-CoV, MERS, COVID-19, and other emerging respiratory viruses.

The term “dispersion”, as used herein, refers to a two-phase system, in which one phase is distributed as droplets in the second, or continuous phase. In these systems, the dispersed phase frequently is referred to as the discontinuous or internal phase, and the continuous phase is called the external phase and comprises a continuous process medium. For example, in course dispersions, the particle size is 0.5 μm. In colloidal dispersions, size of the dispersed particle is in the range of approximately 1 nm to 0.5 μm. A molecular dispersion is a dispersion in which the dispersed phase consists of individual molecules; if the molecules are less than colloidal size, the result is a true solution.

The term “dry powder inhaler” or “DPI” as used herein refers to a device similar to a metered-dose inhaler, but where the drug is in powder form. The patient exhales out a full breath, places the lips around the mouthpiece, and then quickly breathes in the powder. Dry powder inhalers do not require the timing and coordination that are necessary with MDIs.

The term “Drosha” as used herein refers to a nuclear RNase III that cleaves primary miRNAs to release hairpin-shaped pre-miRNAs that are subsequently cut by the cytoplasmic RNase III Dicer to generate mature miRNAs.

The term “Endosomal Sorting Complexes required for transport” (ESCRTs) refers to components involved in multivesicular body (MVB) and intraluminal vesicle (ILV) biogenesis. ESCRTs consist of approximately twenty proteins that assemble into four complexes (ESCRT-0, -I, -II and -III) with associated proteins (VPS4, VTA1, ALIX), which are conserved from yeast to mammals (Colombo, M. et al. J. Cell Science (2013) 126: 5553-65, citing Henne, W. M., et al. (2011). Dev. Cell 21, 77-91; Henne et al. (2011) Roxrud, I. et al. (2010). ESCRT & Co. Biol. Cell 102, 293-318). The ESCRT-0 complex recognizes and sequesters ubiquitylated proteins in the endosomal membrane, whereas the ESCRT-I and -II complexes appear to be responsible for membrane deformation into buds with sequestered cargo, and ESCRT-III components subsequently drive vesicle scission (Id., citing Hurley, J. H. and Hanson, P. I. (2010). Nat. Rev. Mol. Cell Biol. 11, 556-566; Wollert, T. et al. Nature (2009) 458: 172-77). ESCRT-0 comprises HRS protein that recognizes the mono-ubiquitylated cargo proteins and associates in a complex with STAM, Eps15 and clathrin. HRS recruits TSG101 of the ESCRT-I complex, and ESCRT-I is then involved in the recruitment of ESCRT-III, through ESCRT-II or ALIX, an ESCRT-accessory protein. Finally, the dissociation and recycling of the ESCRT machinery requires interaction with the ATPase associated with various cellular activities (AAA-ATPase) Vps4; Vps4 releases ESCRT-III from the MVB membrane for additional sorting events. It is unclear whether ESCRT-II has a direct role in ILV biogenesis or whether its function is limited to particular cargo (Id., citing Bowers, K. et al. (2006) J. Biol. Chem. 281, 5094-5105; Malerod, L. et al. Traffic 8, 1617-1629).

Concomitant depletion of ESCRT subunits belonging to the four ESCRT complexes does not totally impair the formation of MVBs, indicating that other mechanisms may operate in the formation of ILVs and thereby of exosome and or extracellular vesicles (Id., citing Stuffers, S. et al., (2009) Traffic 10, 925-937). One of these pathways requires a type II sphingomyelinase that hydrolyses sphingomyelin to ceramide (Id., citing Trajkovic, K. et al. (2008) Science 319, 1244-1247). Although the depletion of different ESCRT components does not lead to a clear reduction in the formation of MVBs and in the secretion of proteolipid protein (PLP) associated to exosomes and or extracellular vesicles, silencing of neutral sphingomyelinase expression with siRNA or its activity with the drug GW4869 decreases exosome and or extracellular vesicle formation and release. However, whether such dependence on ceramides is generalizable to other cell types producing exosomes and or extracellular vesicles and additional cargos has yet to be determined. The depletion of type II sphingomyelinase in melanoma cells does not impair MVB biogenesis (Id., citing van Niel, G. et al., (2011) Dev. Cell 21, 708-721) or exosome and or extracellular vesicle secretion, but in these cells the tetraspanin CD63 is required for an ESCRT-independent sorting of the luminal domain of the melanosomal protein PMEL (van Niel et al., (2011) Dev. Cell 21, 708-721). Moreover, tetraspanin-enriched domains have been proposed to function as sorting machineries allowing exosome and or extracellular vesicle formation (Perez-Hernandez, D. et al. J. Biol. Chem. (2013) 288, 11649-11661).

Despite evidence for ESCRT-independent mechanisms of exosome and or extracellular vesicle formation, proteomic analyses of purified exosomes and or extracellular vesicles from various cell types have identified ESCRT components (TSG101, ALIX) and ubiquitylated proteins (Id., citing Buschow, S. et al., (2005) Blood Cells Mol. Dis. 35, 398-403; Thery, C. et al., (2006). Curr. Protoc. Cell Biol. Chapter 3, Unit 3.22). It has also been reported that the ESCRT-0 component HRS could be required for exosome and or extracellular vesicle formation and/or secretion by dendritic cells (DCs), and thereby impact on their antigen-presenting capacity (Id., citing Tamai, K. et al., (2010) Biochem. Biophys. Res. Commun. 399, 384-390). The transferrin receptor (TfR) in reticulocytes that is generally fated for exosome and or extracellular vesicle secretion, although not ubiquitylated, interacts with ALIX for MVB sorting (Id., citing Geminard, C. et al., (2004). Traffic 5, 181-193). It was also shown that ALIX is involved in exosome and or extracellular vesicle biogenesis and exosomal sorting of syndecans through its interaction with syntenin (Id., citing Baietti, M F et al., (2012). Nat. Cell Biol. 14, 677-685). Silencing of genes for two components of ESCRT-0 (HRS, STAM1) and one of ESCRT-I (TSG101), as well as a late acting component (VPS4B) induced consistent alterations in exosome and or extracellular vesicle secretion. [Colombo, M. et al. J. Cell Sci. (2013) 126: 5553-65].

As used herein, the term “enrich” is meant to refer to increasing the proportion of a desired substance, for example, to increase the relative frequency of a subtype of cell or cell component compared to its natural frequency in a cell population. Positive selection, negative selection, or both are generally considered necessary to any enrichment scheme. Selection methods include, without limitation, magnetic separation and fluorescence-activated cell sorting (FACS).

Estrogen receptors. Estrogen receptors alpha (ERα) and beta (ERβ) are nuclear transcription factors that are involved in the regulation of many complex physiological processes in humans. They act in the cell nucleus, regulating transcription of specific target genes by binding to associated DNA regulatory sequences. In humans, both receptor subtypes are expressed in many cells and tissues, and they control key physiological functions in various organ systems, such as reproductive, skeletal, cardiovascular and central nervous systems, as well as in specific tissues (such as breast and sub-compartments of prostate and ovary). ERα is present mainly in mammary gland, uterus, ovary (thecal cells), bone, male reproductive organs (testes and epididymis), prostate (stroma), liver, and adipose tissue. ERβ is found mainly in the prostate (epithelium), bladder, ovary (granulosa cells), colon, adipose tissue, and immune system. Both subtypes are markedly expressed in the cardiovascular and central nervous systems. There are some common physiological roles for the two ERs, such as in the development and function of the ovaries, and in the protection of the cardiovascular system. The alpha subtype has a more prominent role on the mammary gland and uterus, as well as on the preservation of skeletal homeostasis and the regulation of metabolism. The beta subtype seems to have a more profound effect on the central nervous and immune systems, and it generally counteracts the ERα-promoted cell hyperproliferation in tissues such as breast and uterus. [Paterni, I. et al. Steroids (2014) 0: 13-29. Doi: 10.1016/j.steroids.2014.06.012].

As used herein, the term “expression” and its various grammatical forms refers to the process by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell. Expression may also refer to the post-translational modification of a polypeptide or protein.

The term “extracellular vesicles” or “EVs” as used herein includes exosomes and microvesicles that carry bioactive molecules, such as proteins, RNAs and microRNAs, that may be released into and influence the extracellular environment. Microvesicles are small membrane-enclosed sacs thought to be generated by the outward budding and fission of membrane vesicles from the cell surface. Exosomes originate predominantly from preformed multivesicular bodies that are released upon fusion with the plasma membrane.

The term “exosomes” as used herein refers to extracellular bilayered membrane-bound vesicles of endosomal origin in a size range of ˜40 to 160 nm in diameter (˜100 nm on average) generated by all cells that are actively secreted.

When used to describe the expression of a gene or polynucleotide sequence, the terms “down-regulation”, “disruption”, “inhibition”, “inactivation”, and “silencing” are used interchangeably herein to refer to instances when the transcription of the polynucleotide sequence is reduced or eliminated. This results in the reduction or elimination of RNA transcripts from the polynucleotide sequence, which results in a reduction or elimination of protein expression derived from the polynucleotide sequence (if the gene comprised an ORF). Alternatively, down-regulation can refer to instances where protein translation from transcripts produced by the polynucleotide sequence is reduced or eliminated. Alternatively still, down-regulation can refer to instances where a protein expressed by the polynucleotide sequence has reduced activity. The reduction in any of the above processes (transcription, translation, protein activity) in a cell can be by about 20%, 30%, 40%, 50%, 60%, 70%, 80%, 90%, 95%, or 100% relative to the transcription, translation, or protein activity of a suitable control cell. Down-regulation can be the result of a targeting event as disclosed herein (e.g., indel, knock-out), for example.

The term “clinical efficacy” as used herein refers to the therapeutic effectiveness of a drug or therapeutic in humans using appropriate outcome measures.

The term “extracellular matrix” as used herein refers to a scaffold in a cell's external environment with which the cell interacts via specific cell surface receptors. The extracellular matrix serves many functions, including, but not limited to, providing support and anchorage for cells, segregating one tissue from another tissue, and regulating intracellular communication. The extracellular matrix is composed of an interlocking mesh of fibrous proteins and glycosaminoglycans (GAGs). Examples of fibrous proteins found in the extracellular matrix include collagen, elastin, fibronectin, and laminin. Examples of GAGs found in the extracellular matrix include proteoglycans (e.g., heparin sulfate), chondroitin sulfate, keratin sulfate, and non-proteoglycan polysaccharide (e.g., hyaluronic acid). The term “proteoglycan” refers to a group of glycoproteins that contain a core protein to which is attached one or more glycosaminoglycans.

The term “extracellular vesicles (EVs)” as used herein refers to nanosized, membrane-bound vesicles released from cells that can transport cargo-including DNA, RNA, and proteins-between cells as a form of intercellular communication. Different EV types, including microvesicles (MVs), exosomes, oncosomes, and apoptotic bodies, have been characterized on the basis of their biogenesis or release pathways. Microvesicles bud directly from the plasma membrane, are 100 nanometers (nm) to 1 micrometer (μm) in size, and contain cytoplasmic cargo (Zaborowski, M P et al. BioScience (2015) 65 (8): 783-97, citing Heijnen, H F et al. Blood (1999) 94: 3791-99). Another EV subtype, exosomes, is formed by the fusion between multivesicular bodies and the plasma membrane, by which multivesicular bodies release smaller vesicles (exosomes) whose diameters range from 40 to 120 nm (Id., citing El Andaloussi, S. et al. Nature Reviews Drug Discovery (2013) 12: 347-57; Cocucci, E. and Meldolesi J. Trends in Cell Biology (2015) 25: 364-72). Dying cells, release vesicular apoptotic bodies (50 nm-2 m) that can be more abundant than exosomes and or extracellular vesicles or MVs under specific conditions and can vary in content between biofluids (Id., citing Thery, C. et al. J. Immunology (2001) 1666: 7309-18; El Andaloussi, S. et al. Nature Reviews Drug Discovery (2013) 12: 347-57). Membrane protrusions can also give rise to large EVs, termed oncosomes (1-10 m), which are produced primarily by malignant cells in contrast to their nontransformed counterparts (Id., citing Di Vizio, D. et al. Am. J. Pathol. (2012) 181: 1573-84; Morello, M. et al. Cell Cycle (2013) 12: 3526-36).

The term “forced expiratory volume” or “FEV1” as used herein refers to the volume of air that an individual can exhale during a forced breath in 1 second. The normal value (95% confidence interval) for FEV1 is 80% to 120%.

The term “forced vital capacity” or “FVC” as used herein refers to the maximal volume of gas that can be exhaled from full inhalation by exhaling as forcefully and rapidly as possible. The normal value (95% confidence interval) for FVC is 80% to 120%.

The FEV1/FVC ratio or Tiffeneau Index is the amount of air exhaled in the first second divided by all of the air exhaled during a maximal exhalation. The ratio FEV1/FVC is between 70% and 80% in normal adults; a value less than 70% indicates airflow limitation.

The term “free radical” is defined as any chemical species capable of independent existence that contains one or more unpaired electrons. An unpaired electron refers to the one that occupies an atomic or molecular orbital by itself. Examples of oxygen radicals include superoxide, hydroxyl, peroxyl, and alkoxyl radicals.

The term “growth factor” as used herein refers to extracellular polypeptide molecules that bind to a cell-surface receptor triggering an intracellular signaling pathway, leading to proliferation, differentiation, or other cellular response that stimulate the accumulation of proteins and other macromolecules, e.g., by increasing their rate of synthesis, decreasing their rate of degradation, or both. Exemplary growth factors include fibroblast growth factor (FGF), insulin-like growth factor (IGF-1), transforming growth factor beta (TGF-β), and vascular endothelial growth factor (VEGF)

Fibroblast Growth Factor (FGF). The fibroblast growth factor (FGF) family currently has over a dozen structurally related members. FGF1 is also known as acidic FGF; FGF2 is sometimes called basic FGF (bFGF); and FGF7 sometimes goes by the name keratinocyte growth factor. Over a dozen distinct FGF genes are known in vertebrates; they can generate hundreds of protein isoforms by varying their RNA splicing or initiation codons in different tissues. FGFs can activate a set of receptor tyrosine kinases called the fibroblast growth factor receptors (FGFRs). Receptor tyrosine kinases are proteins that extend through the cell membrane. The portion of the protein that binds the paracrine factor is on the extracellular side, while a dormant tyrosine kinase (i.e., a protein that can phosphorylate another protein by splitting ATP) is on the intracellular side. When the FGF receptor binds an FGF (and only when it binds an FGF), the dormant kinase is activated, and phosphorylates certain proteins within the responding cell, activating those proteins.

FGFs are associated with several developmental functions, including angiogenesis (blood vessel formation), mesoderm formation, and axon extension. While FGFs often can substitute for one another, their expression patterns give them separate functions. For example, FGF2 is especially important in angiogenesis, whereas FGF8 is involved in the development of the midbrain and limbs.

The term “hepatocyte growth factor” (or HGF) as used herein refers to a pleiotrophic growth factor, which induces cellular motility, survival, proliferation, and morphogenesis, depending upon the cell type. In the adult, HGF has been demonstrated to play a critical role in tissue repair, including in the lung. Administration of HGF protein or ectopic expression of HGF has been demonstrated in animal models of pulmonary fibrosis to induce normal tissue repair and to prevent fibrotic remodeling. HGF-induced inhibition of fibrotic remodeling may occur via multiple direct and indirect mechanisms including the induction of cell survival and proliferation of pulmonary epithelial and endothelial cells, and the reduction of myofibroblast accumulation. [Panganiban, R A M and Day, R M, Acta Pharmacol. Sin. (2011) 32 (1): 12-20].

Insulin-Like Growth Factor (IGF-1). IGF-1, a hormone similar in molecular structure to insulin, has growth-promoting effects on almost every cell in the body, especially skeletal muscle, cartilage, bone, liver, kidney, nerves, skin, hematopoietic cell, and lungs. It plays an important role in childhood growth and continues to have anabolic effects in adults. IGF-1 is produced primarily by the liver as an endocrine hormone as well as in target tissues in a paracrine/autocrine fashion. Production is stimulated by growth hormone (GH) and can be retarded by undernutrition, growth hormone insensitivity, lack of growth hormone receptors, or failures of the downstream signaling molecules, including tyrosine-protein phosphatase non-receptor type 11 (also known as SHP2, which is encoded by the PTPN11 gene in humans) and signal transducer and activator of transcription 5B (STAT5B), a member of the STAT family of transcription factors. Its primary action is mediated by binding to its specific receptor, the Insulin-like growth factor 1 receptor (IGF1R), present on many cell types in many tissues. Binding to the IGF1R, a receptor tyrosine kinase, initiates intracellular signaling; IGF-1 is one of the most potent natural activators of the AKT signaling pathway, a stimulator of cell growth and proliferation, and a potent inhibitor of programmed cell death. IGF-1 is a primary mediator of the effects of growth hormone (GH). Growth hormone is made in the pituitary gland, released into the blood stream, and then stimulates the liver to produce IGF-1. IGF-1 then stimulates systemic body growth. In addition to its insulin-like effects, IGF-1 also can regulate cell growth and development, especially in nerve cells, as well as cellular DNA synthesis.

IGF-1 was shown to increase the expression levels of the chemokine receptor CXCR4 (receptor for stromal cell-derived factor-1, SDF-1) and to markedly increase the migratory response of MSCs to SDF-1 (Li, Y, et al. 2007 Biochem. Biophys. Res. Communic. 356(3): 780-784). The IGF-1-induced increase in MSC migration in response to SDF-1 was attenuated by PI3 kinase inhibitor (LY294002 and wortmannin) but not by mitogen-activated protein/ERK kinase inhibitor PD98059. Without being limited by any particular theory, the data indicate that IGF-1 increases MSC migratory responses via CXCR4 chemokine receptor signaling which is PI3/Akt dependent.

The term “platelet derived growth factor” or “PDGF” as used herein refers to a major mitogen for connective tissue cells and certain other cell types. It is a dimeric molecule consisting of disulfide-bonded, structurally similar A- and B-polypeptide chains, which combine to homo- and heterodimers. The PDGF isoforms exert their cellular effects by binding to and activating two structurally related protein tyrosine kinase receptors, denoted the alpha-receptor and the beta-receptor. Activation of PDGF receptors leads to stimulation of cell growth, but also to changes in cell shape and motility; PDGF induces reorganization of the actin filament system and stimulates chemotaxis, i.e., a directed cell movement toward a gradient of PDGF. In vivo, PDGF has important roles during the embryonic development as well as during wound healing. Moreover, overactivity of PDGF has been implicated in several pathological conditions. Helden, C H and Westermark, B. Physiol. Rev. (1999) 79 (4): 1283-316].

Transforming Growth Factor Beta (TGF-β). There are over 30 structurally related members of the TGF-β superfamily, and they regulate some of the most important interactions in development. The proteins encoded by TGF-β superfamily genes are processed such that the carboxy-terminal region contains the mature peptide. These peptides are dimerized into homodimers (with themselves) or heterodimers (with other TGF-β peptides) and are secreted from the cell. The TGF-β superfamily includes the TGF-β family, the activin family, the bone morphogenetic proteins (BMPs), the Vg-1 family, and other proteins, including glial-derived neurotrophic factor (GDNF, necessary for kidney and enteric neuron differentiation) and Müllerian inhibitory factor, which is involved in mammalian sex determination. TGF-β family members TGF-β1, 2, 3, and 5 are important in regulating the formation of the extracellular matrix between cells and for regulating cell division (both positively and negatively). TGF-β1 increases the amount of extracellular matrix epithelial cells make both by stimulating collagen and fibronectin synthesis and by inhibiting matrix degradation. TGF-βs may be critical in controlling where and when epithelia can branch to form the ducts of kidneys, lungs, and salivary glands.

The term “tumor necrosisfactor-alpha” (“TNF-α”) as used herein refers to a potent pro-inflammatory cytokine exerting pleiotropic effects on various cell types and plays a critical role in the pathogenesis of chronic inflammatory diseases, Transmembrane TNF-α, a precursor of the soluble form of TNF-α, is expressed on activated macrophages and lymphocytes as well as other cell types. After processing by TNF-α-converting enzyme (TACE), the soluble form of TNF-α is cleaved from transmembrane TNF-α and mediates its biological activities through binding to Types 1 and 2 TNF receptors (TNF-R1 and -R2) of remote tissues. Accumulating evidence suggests that not only soluble TNF-α, but also transmembrane TNF-α is involved in the inflammatory response. [Horiuchi, T. et al. Rheumatology (Oxford)](2010) 49 (7): 1215-28].

Vascular Endothelial Growth Factor (VEGF). VEGFs are growth factors that mediate numerous functions of endothelial cells including proliferation, migration, invasion, survival, and permeability. The VEGFs and their corresponding receptors are key regulators in a cascade of molecular and cellular events that ultimately lead to the development of the vascular system, either by vasculogenesis, angiogenesis, or in the formation of the lymphatic vascular system. VEGF is a critical regulator in physiological angiogenesis and also plays a significant role in skeletal growth and repair.

VEGF's normal function creates new blood vessels during embryonic development, after injury, and to bypass blocked vessels. In the mature established vasculature, the endothelium plays an important role in the maintenance of homeostasis of the surrounding tissue by providing the communicative network to neighboring tissues to respond to requirements as needed. Furthermore, the vasculature provides growth factors, hormones, cytokines, chemokines and metabolites, and the like, needed by the surrounding tissue and acts as a barrier to limit the movement of molecules and cells.

The term “healthy control” as used herein refers to a subject in a state of physical well-being without signs or symptoms of a fibrotic lung disease or process.

Hydroxyproline assay. Collagen content is assessed by quantifying hydroxyproline, an amino acid present in appreciable quantities in collagen.

The term “high throughput screening” or “HTS” as used herein refers to the use of automated equipment to rapidly test thousands to millions of samples for biological activity at the model organism, cellular, pathway, or molecular level.

The term “immune system” as used herein refers to a complex arrangement of cells and molecules that maintain immune homeostasis to preserve the integrity of the organism by elimination of all elements judged to be dangerous. Responses in the immune system may generally be divided into two arms, referred to as “innate immunity” and “adaptive immunity.” The two arms of immunity do not operate independently of each other, but rather work together to elicit effective immune responses.

The term “inflammation” as used herein refers to the physiologic process by which vascularized tissues respond to injury. See, e.g., FUNDAMENTAL IMMUNOLOGY, 4th Ed., William E. Paul, ed. Lippincott-Raven Publishers, Philadelphia (1999) at 1051-1053, incorporated herein by reference. During the inflammatory process, cells involved in detoxification and repair are mobilized to the compromised site by inflammatory mediators. Inflammation is often characterized by a strong infiltration of leukocytes at the site of inflammation, particularly neutrophils (polymorphonuclear cells). These cells promote tissue damage by releasing toxic substances at the vascular wall or in uninjured tissue. Traditionally, inflammation has been divided into acute and chronic responses.

The term “acute inflammation” as used herein refers to the rapid, short-lived (minutes to days), relatively uniform response to acute injury characterized by accumulations of fluid, plasma proteins, and neutrophilic leukocytes. Examples of injurious agents that cause acute inflammation include, but are not limited to, pathogens (e.g., bacteria, viruses, parasites), foreign bodies from exogenous (e.g. asbestos) or endogenous (e.g., urate crystals, immune complexes), sources, and physical (e.g., burns) or chemical (e.g., caustics) agents.

The term “chronic inflammation” as used herein refers to inflammation that is of longer duration and which has a vague and indefinite termination. Chronic inflammation takes over when acute inflammation persists, either through incomplete clearance of the initial inflammatory agent or as a result of multiple acute events occurring in the same location. Chronic inflammation, which includes the influx of lymphocytes and macrophages and fibroblast growth, may result in tissue scarring at sites of prolonged or repeated inflammatory activity. The term “innate immunity” as used herein refers to a nonspecific fast response to pathogens that is predominantly responsible for an initial inflammatory response before adaptive immunity is induced. These include such mechanisms as anatomical barriers, antimicrobial peptides, the complement system and the chemokine/cytokine system; macrophages and neutrophils carrying nonspecific pathogen-recognition receptors, and a number of specialized cell types, including innate lymphoid cells (TLCs, including natural killer (NK) cells) mast cells and dendritic cells (DCs). Innate immunity is present in all individuals at all times, does not increase with repeated exposure to a given pathogen, and discriminates between groups of similar pathogens, rather than responding to a specific pathogen.

The term “infuse” and its other grammatical forms as used herein refers to introduction of a fluid other than blood into a vein.

The term “inhalation delivery device” as used herein refers to a machine/apparatus or component that produces small droplets or an aerosol from a liquid or dry powder aerosol formulation and is used for administration through the mouth in order to achieve pulmonary administration of a drug, e.g., in solution, powder, and the like. Examples of inhalation delivery device include, but are not limited to, a nebulizer, a metered-dose inhaler, and a dry powder inhaler (DPI).

The terms “inhibiting”, “inhibit” or “inhibition” are used herein to refer to reducing the amount or rate of a process, to stopping the process entirely, or to decreasing, limiting, or blocking the action or function thereof. Inhibition may include a reduction or decrease of the amount, rate, action function, or process of a substance by at least about 5%, at least about 10%, at least about 15%, at least about 20%, at least about 25%, at least about 30%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, or at least about 99%.

The term “inhibitor” as used herein refers to a molecule that reduces the amount or rate of a process, stops the process entirely, or that decreases, limits, or blocks the action or function thereof. Enzyme inhibitors are molecules that bind to enzymes thereby decreasing enzyme activity. Inhibitors may be evaluated by their specificity and potency.

The term “insufflation” as used herein refers to the act of delivering air, a gas, or a powder under pressure to a cavity or chamber of the body. For example, nasal insufflation relates to the act of delivering air, a gas, or a powder under pressure through the nose.

The term “interleukin” as used herein refers to a cytokine secreted by white blood cells as a means of communication with other white blood cells. For example, interleukin-8 (or “IL-8”) is produced by phagocytes and mesenchymal cells exposed to inflammatory stimuli (e.g., interleukin-1 or tumor necrosis factor) and activates neutrophils inducing chemotaxis, exocytosis and the respiratory burst. In vivo, IL-8 elicits a massive neutrophil accumulation at the site of injection. Interleukin (IL)-2, IL-4, IL-7, IL-9, IL-15, and IL-21 form a family of cytokines based on their sharing the common cytokine receptor γ chain (γc). IL-2 and IL-15 both share IL-2Rβ and γc and activate the same Janus kinase (JAK)1/JAK3/signal transducers and activators of transcription (STAT)5 pathway. Although many different cell types can express IL-15 messenger RNA (mRNA), IL-15 protein is mainly produced by dendritic cells (DCs) and monocytes in response to Toll-like receptor (TLR) activation and binds to receptors on these cells. IL-15 plays critical roles in the development and/or maintenance of memory CD8+ T cells and preferentially can expand central memory phenotype T cells in vivo, with Il 15−/− or Il15ra−/− mice having profound loss of memory phenotype CD8+ T cells, intestinal intraepithelial lymphocytes, NKT cells, and NK cells. IL-15 receptors are composed of IL-15Rα, IL-2Rβ, and γc. IL-15Rα is expressed on a wide range of cells, including immune cells (T cells, B cells, macrophages, and stromal-cell lines) and nonimmune cells (keratinocytes and skeletal muscle cells). IL-15 binds to IL-15Rα with high affinity (K_{d}≈25 pm), much higher than the affinity of IL-2 for IL-2Rα, whereas IL-15 binds IL-2Rβ and γc with a K_{d}≈1 nm, similar to the affinity of IL-2 to IL-2Rβ and γc. Like IL-2Rα, IL-15Rα does not transduce IL-15 signals, but IL-15Rα-expressing APCs, such as DCs and monocytes, can bind IL-15 and trans-present the cytokine to lymphocytes that express IL-2Rβ and 7c; this trans signaling is the dominant mode for IL-15 action. [Lin, J-X and Leonard, WJ. Cold Spring Harb. Persp. Biol. 2018] 10 (9): a028449]

The term “interleukin-1 receptor associated kinase” or IRAK-1″ IRAK-4, refer to protein kinases that are part of the intracellular signaling pathways leading from TLRs.

The term “invasive” and its other grammatical forms as used herein means requiring medical instruments to enter or penetrate the body or to disturb body tissue. The term “noninvasive” and its various grammatical forms accordingly means not requiring medical instruments to enter or penetrate the body or to disturb body tissue.

The term “isolated” is used herein to refer to material, such as, but not limited to, a nucleic acid, peptide, polypeptide, or protein, which is: (1) substantially or essentially free from components that normally accompany or interact with it as found in its naturally occurring environment. The terms “substantially free” or “essentially free” are used herein to refer to considerably or significantly free of, or more than about 95%, 96%, 97%, 98%, 99% or 100% free. The isolated material optionally comprises material not found with the material in its natural environment; or (2) if the material is in its natural environment, the material has been synthetically (non-naturally) altered by deliberate human intervention to a composition and/or placed at a location in the cell (e.g., genome or subcellular organelle) not native to a material found in that environment. The alteration to yield the synthetic material may be performed on the material within, or removed, from its natural state.

The term “JAG1” refers to the gene that encodes protein jagged-1. Jagged-1 is the ligand for multiple Notch receptors and is involved in the mediation of Notch signaling.

The term “long noncoding RNA” (“lncRNAs”) as used herein refers to a class of transcribed RNA molecules that are longer than 200 nucleotides and yet do not encode proteins. LncRNAs can fold into complex structures and interact with proteins, DNA and other RNAs, modulating the activity, DNA targets or partners of multiprotein complexes. Crosstalk of lncRNAs with miRNAs creates an intricate network that exerts post-transcriptional regulation of gene expression. For example, lncRNAs can harbor miRNA binding sites and act as molecular decoys or sponges that sequester miRNAs away from other transcripts. Competition between lncRNAs and miRNAs for binding to target mRNAs has been reported and leads to de-repression of gene expression (Zampetaki, A. et al. Front. Physiol. (2018) doi.org/10.3389/fphys.2018.01201, citing Yoon, J H et al. Semin. Cell Dev. Bio. (2014) 34: 9-14; Ballantyne, M D et al. Clin. Pharmacol. Ther. (2016) 99: 494-501). Finally, lncRNAs may contain embedded miRNA sequences and serve as a source of miRNAs (Id., citing Piccoli, M T et al. Cir. Res. (2017) 121: 575-83).

The terms “lung function” or “pulmonary function” are used interchangeably to refer to the process of gas exchange called respiration (or breathing). In respiration, oxygen from incoming air enters the blood, and carbon dioxide, a waste gas from the metabolism, leaves the blood. A reduced lung function means that the ability of lungs to exchange gases is reduced.

The terms “lung interstitium” or “pulmonary interstitium” are used interchangeably herein to refer to an area located between the airspace epithelium and pleural mesothelium in the lung. Fibers of the matrix proteins, collagen and elastin, are the major components of the pulmonary interstitium. The primary function of these fibers is to form a mechanical scaffold that maintains structural integrity during ventilation.

The abbreviation “MAPK” as used herein refers to Mitogen-Activated Protein Kinase (MAPK) signaling which activates a three-tiered cascade with MAPK kinase kinases (MAP3K) activating MAPAK kinases (MAP2K) and finally MAPK. MAPKs are protein Ser/Thr kinases that convert extracellular stimuli into a wide range of cellular responses. (Cargnello, M. and Roux, PP, Microbiol. Mol. Biol. Rev. (2011) 75(1): 50-83). The major MAPK pathways involved in inflammatory diseases are extracellular regulating kinase (ERK), p38 MAPK, and c-Jun NH2-terminal kinase (INK). All three MAPK pathways may be activated by TGF-β, and signaling through these cascades can further regulate the expression of Smad proteins and mediate Smad-independent TGF-β responses. These three MAPK pathways are all involved in TGF-β-induced fibrosis. [He, W. and Dai, C. Curr. Pathobiol. Rep. (2015) 3: 183-92, citing Tsou, P S et al. Am. J. Physiol. Cell Physiol. (22014) 307: C2-13; Kamato, D. et al. Cell Signal (2013) 25: 2017-24; Pannu, J. et al. J. Biol. Chem. (2007) 282: 10405-13; Yu, L. et al. J. Biol. Chem. (2002) EMBO J. 21: 3749-59].

Upstream kinases include TGFβ-activated kinase-1 (TAK1) and apoptosis signal-regulating kinase-1 (ASK1). Downstream of p38 MAPK is MAPK activated protein kinase 2 (MAPKAPK2 or MK2). TGF-β can signal in a noncanonical manner via the MAPK family.

The term “matrix metalloproteinases” as used herein refers to a collection of zinc-dependent proteases involved in the breakdown and the remodeling of extracellular matrix components (Guiot, J. et al. Lung (2017) 195(3): 273-280, citing Oikonomidi et al. Curr Med Chem. 2009; 16(10): 1214-1228). MMP-1 and MMP-7 seem to be primarily overexpressed in plasma of IPF patients compared to hypersensitivity pneumonitis, sarcoidosis and COPD with a possible usefulness in differential diagnosis (Id., citing Rosas I O, et al. PLoS Med. 2008; 5(4): e93). They are also involved in inflammation and seem to take part to the pathophysiological process of pulmonary fibrosis (Id., citing Vij R, Noth I. Transl Res. 2012; 159(4): 218-27; Dancer RCA, et al. Eur Respir J. 2011; 38(6): 1461-67). The most studied is MMP-7, which is known as being significantly increased in epithelial cells both at the gene and protein levels and is considered to be active in hyperplastic epithelial cells and alveolar macrophages in IPF (Id., citing Fujishima S, et al. Arch Pathol Lab Med. 2010; 134(8): 1136-42). There is also a significant correlation between higher MMP-7 concentrations and disease severity assessed by forced vital capacity (FVC) and diffusing capacity of the lungs for carbon monoxide (DLCO) (Id., citing Rosas I O, et al. PLoS Med. 2008; 5(4): e93). Higher levels associated to disease progression and worse survival (>4.3 ng/ml for MMP-7) (Id.). The MMP2 gene provides instructions for making matrix metallopeptidase 2. This enzyme is produced in cells throughout the body and becomes part of the extracellular matrix, which is an intricate lattice of proteins and other molecules that forms in the spaces between cells. One of the major known functions of MMP-2 is to cleave type IV collagen, which is a major structural component of basement membranes, the thin, sheet-like structures that separate and support cells as part of the extracellular matrix.

MMPs play a critical role in neuroinflammation through the cleavage of ECM proteins, cytokines and chemokines. (Ji. R-R et al, US Neurology, Touch Briefings (2008) 71-74). MMP-2 is constitutively expressed and normally present in brain and spinal cord tissues. In contrast, MMP-9 is normally expressed at low levels, but upregulated in many injury and disease states such as spinal cord injury and brain trauma (Id., citing Rosenberg, G A. Glia (2002) 39: 279-91); it is also induced in the crushed sciatic nerve and causes demyelination, a condition associated with neuropathic pain, by the cleavage of myelin basic protein. (Id., citing Chattopadhyay, S. et al. Brain Behav. Immun. (20007) 21: 561-8). Besides targeting matrix, because MMPs can process a variety of growth factors and other extracellular cytokines and signals, they may contribute to the neurovascular remodeling that accompanies chronic CNS injury. (Id., citing Zhao, B Q, et al. Nat. Med. (2006) 12: 441-45).

The term “mesenchymal stem cells” (MSCs) (also known as bone marrow stromal stem cells or skeletal stem cells) are non-blood adult stem cells found in a variety of tissues. They are characterized by their spindle-shape morphologically; by the expression of specific markers on their cell surface; and by their ability, under appropriate conditions, to differentiates along a minimum of three lineages (osteogenic, chondrogenic, and adipogenic) [Najar M. et al., “Mesenchymal stromal cells and immunomodulation: A gathering of regulatory immune cells”, Cytotherapy, Vol. 18(2): 160-171, (2016)]. No single marker that definitely delineates MSCs in vivo has been identified due to the lack of consensus regarding the MSC phenotype, but it generally is considered that MSCs are positive for cell surface markers CD105, CD166, CD90, and CD44 and that MSCs are negative for typical hematopoietic antigens, such as CD45, CD34, and CD14. As for the differentiation potential of MSCs, studies have reported that populations of bone marrow-derived MSCs have the capacity to develop into terminally differentiated mesenchymal phenotypes both in vitro and in vivo, including bone, cartilage, tendon, muscle, adipose tissue, and hematopoietic supporting stroma. Studies using transgenic and knockout mice and human musculoskeletal disorders have reported that MSC differentiate into multiple lineages during embryonic development and adult homeostasis [Najar M. et al., “Mesenchymal stromal cells and immunomodulation: A gathering of regulatory immune cells”, Cytotherapy, Vol. 18(2): 160-171, (2016)].

The term “metered-dose inhaler”, “MDI”, or “puffer” as used herein refers to a pressurized, hand-held device that uses propellants to deliver a specific amount of medicine (“metered dose”) to the lungs of a patient. The term “propellant” as used herein refers to a material that is used to expel a substance usually by gas pressure through a convergent, divergent nozzle. The pressure may be from a compressed gas, or a gas produced by a chemical reaction. The exhaust material may be a gas, liquid, plasma, or, before the chemical reaction, a solid, liquid or gel. Propellants used in pressurized metered dose inhalers are liquefied gases, traditionally chlorofluorocarbons (CFCs) and increasingly hydrofluoroalkanes (HFAs). Suitable propellants include, for example, a chlorofluorocarbon (CFC), such as trichlorofluoromethane (also referred to as propellant 11), dichlorodifluoromethane (also referred to as propellant 12), and 1,2-dichloro-1,1,2,2-tetrafluoroethane (also referred to as propellant 114), a hydrochlorofluorocarbon, a hydrofluorocarbon (HFC), such as 1,1,1,2-tetrafluoroethane (also referred to as propellant 134a, HFC-134a, or HFA-134a) and 1,1,1,2,3,3,3-heptafluoropropane (also referred to as propellant 227, HFC-227, or HFA-227), carbon dioxide, dimethyl ether, butane, propane, or mixtures thereof. In other embodiments, the propellant includes a chlorofluorocarbon, a hydrochlorofluorocarbon, a hydrofluorocarbon, or mixtures thereof. In other embodiments, a hydrofluorocarbon is used as the propellant. In other embodiments, HFC-227 and/or HFC-134a are used as the propellant.

The term “microRNA” (or “miRNA” or “miR”) as used herein refers to a class of small, 18- to 28-nucleotide-long, noncoding RNA molecules.

The term “modulate” as used herein means to regulate, alter, adapt, or adjust to a certain measure or proportion.

The terms “monitor” or “clinically monitor” are used interchangeably herein to refer to a process of watching so as to provide a warning of the occurrence of events, operations or circumstances.

As used herein the term “natural killer (NK) cells” refers to lymphocytes in the same family as T and B cells, classified as group I innate lymphocytes. They have an ability to kill tumor cells without any priming or prior activation, in contrast to cytotoxic T cells, which need priming by antigen presenting cells. NK cells secrete cytokines such as IFNγ and TNFα, which act on other immune cells, like macrophages and dendritic cells, to enhance the immune response. Activating receptors on the NK cell surface recognize molecules expressed on the surface of cancer cells and infected cells and switch on the NK cell. Inhibitory receptors act as a check on NK cell killing. Most normal healthy cells express MHCI receptors, which mark them as “self.” Inhibitory receptors on the surface of the NK cell recognize cognate MHCI, which switches off the NK cell, preventing it from killing. Once the decision is made to kill, the NK cell releases cytotoxic granules containing perforin and granzymes, which leads to lysis of the target cell. Natural killer reactivity, including cytokine secretion and cytotoxicity, is controlled by a balance of several germ-line encoded inhibitory and activating receptors such as killer immunoglobulin-like receptors (KIRs) and natural cytotoxicity receptors (NCRs). The presence of the MHC Class I molecule on target cells serves as one such inhibitory ligand for MHC Class I-specific receptors, the Killer cell Immunoglobulin-like Receptor (KIR), on NK cells. Engagement of KIR receptors blocks NK activation and, paradoxically, preserves their ability to respond to successive encounters by triggering inactivating signals. Therefore, if a KIR is able to sufficiently bind to MHC Class I, this engagement may override the signal for killing and allows the target cell to live. In contrast, if the NK cell is unable to sufficiently bind to MHC Class I on the target cell, killing of the target cell may proceed. Consequently, those tumors which express low MHC Class I and which are thought to be capable of evading a T-cell-mediated attack may be susceptible to an NK cell-mediated immune response instead.

The abbreviation “NFκB” as used herein refers to which is a proinflammatory transcription factor. It switches on multiple inflammatory genes, including cytokines, chemokines, proteases, and inhibitors of apoptosis, resulting in amplification of the inflammatory response [Barnes, PJ, (2016) Pharmacol. Rev. 68: 788-815]. The molecular pathways involved in NF-1B activation include several kinases. The classic (canonical) pathway for inflammatory stimuli and infections to activate NF-κB signaling involve the IKK (inhibitor of KB kinase) complex, which is composed of two catalytic subunits, IKK-α and IKK-β, and a regulatory subunit IKK-γ (or NFκB essential modulator [Id., citing Hayden, M S and Ghosh, S (2012) Genes Dev. 26: 203-234]. The IKK complex phosphorylates Nf-κB-bound IκBs, targeting them for degradation by the proteasome and thereby releasing NF-κB dimers that are composed of p65 and p50 subunits, which translocate to the nucleus where they bind to KB recognition sites in the promoter regions of inflammatory and immune genes, resulting in their transcriptional activation. This response depends mainly on the catalytic subunit IKK-β (also known as IKK2), which carries out IκB phosphorylation. The noncanonical (alternative) pathway involves the upstream kinase NF-κB-inducing kinase (NTK) that phosphorylates IKK-α homodimers and releases RelB and processes p100 to p52 in response to certain members of the TNF family, such as lymphotoxin-β [Id., citing Sun, S C. (2012) Immunol. Rev. 246: 125-140]. This pathway switches on different gene sets and may mediate different immune functions from the canonical pathway. Dominant-negative IKK-β inhibits most of the proinflammatory functions of NF-κB, whereas inhibiting IKK-α has a role only in response to limited stimuli and in certain cells such as B-lymphocytes. The noncanonical pathway is involved in development of the immune system and in adaptive immune responses. The coactivator molecule CD40, which is expressed on antigen-presenting cells, such as dendritic cells and macrophages, activates the noncanonical pathway when it interacts with CD40L expressed on lymphocytes [Id., citing Lombardi, V et al. (2010) Int. Arch. Allergy Immunol. 151: 179-89].

The term “Notch” refers to a signaling pathway that has been implicated in abnormal differentiation of respiratory epithelial cells in progressive IPF or secondary pulmonary fibrosis. [He, W. and Dai, C. Curr. Pathobiol. Resp. (2015) 3: 183-92, citing Plantier, L. et al. Thorax (2011) 66: 651-57]. Notch proteins are single-pass transmembrane receptors with conserved expression among animal species during evolution. Their principal function is the regulation of many developmental processes, including proliferation, differentiation, and apoptosis. Mammals possess four different Notch receptors, referred to as Notch 1-4. The Notch receptor consists of an extracellular domain, which is involved in ligand binding, and an intracellular domain that works in signal transduction. Notch ligands also are single-pass transmembrane proteins named Jagged (Jag1 and 2) and Delta (Dll1, 3, and 4) [Id., citing Sharma, S. et al. Curr. Opin. Nephrol Hypertens. (2011) 20: 56-61; Bray, SJ. Nat. Rev. Mol. Cell Biol. (2006) 7: 678-89]. Activation of this signaling pathway requires cell-cell contact. Interaction of ligands with the Notch receptors triggers a series of proteolytic cleavages, by a metalloprotease of the ADAM family (TACE; tumor necrosis factor-α-converting enzyme) and finally by the γ-secretase complex. The final cleavage leads to the release of Notch intracellular domain (NICD), which travels to the nucleus and binds to other transcriptional regulators (mainly of the CBF1/RBP-Jκ, SU(H), Lagl family) to trigger the transcription of the target genes, classically belonging to the Hes and Hey family. This core signal transduction pathway is used in most Notch-dependent processes and is known as the canonical Notch pathway [Id., citing Sharma, S. et al. Curr. Opin. Nephrol. Hypertens. (2011) 20: 56-61; Kavian N. et al. Open Rheumatol. J (2012) 6: 96-102; Fortini, M E. Dev. Cell (2009) 16: 633-47]. During the past few years, activation of Notch signaling has shown fibrogenic effects in a wide spectrum of diseases, including systemic sclerosis (SSc) [Id., citing Dees, C. et al. Ann. Rheum. Dis. (2011) 70: 1304-10; Kavian, N. et al. Arthritis Rheum. (2010) 62: 3477-87], scleroderma, idiopathic pulmonary fibrosis (IPF) [Id., citing Plantier, L. et al. Thorax (2011) 66: 651-7], kidney fibrosis [Id., citing Sharma, S. et al. Curr. Opin. Nephrol. Hpertens. (2011) 20: 56-61; Niranjan, T. et al. Nat. Med. (2008) 14: 290-98; Murea, M. et al. Kidney Int. (2010) 78: 514-22], and cardiac fibrosis [Id., citing Kavian, N. et al. Open Rheumatol J. (2012) 6: 96-102].

The term “organ” as used herein refers to a differentiated structure consisting of cells and tissues and performing some specific function in an organism.

The term “oxidative stress” as used herein refers to a condition where the levels of ROS significantly overwhelm the capacity of antioxidant defenses, leading to potential damage in a biological system. Oxidative stress condition can be caused by either increased ROS formation or decreased activity of antioxidants or both. not any increases in ROS levels in a biological system are associated with injury. Under certain circumstances, small transient increases in ROS levels can be employed as a signaling mechanism, leading to physiological cellular responses. [Li, R. et al. React. Oxyg. Species (Apex) (2016) 1 (1): 9-21].

As used herein, the term “paracrine signaling” refers to short range cell-cell communication via secreted signal molecules that act on adjacent cells.

The term “parenteral” as used herein refers to introduction into the body by way of an injection (i.e., administration by injection), including, for example, subcutaneously (i.e., an injection beneath the skin), intramuscularly (i.e., an injection into a muscle), intravenously (i.e., an injection into a vein), intrathecally (i.e., an injection into the space around the spinal cord or under the arachnoid membrane of the brain), intrasternal injection or infusion techniques.

The term “particles” as used herein refers to refers to an extremely small constituent (e.g., nanoparticles, microparticles, or in some instances larger) in or on which is contained the composition as described herein.

The term “particulate” as used herein refers to fine particles of solid or liquid matter suspended in a gas or liquid.

The term “pathogen” as used herein refers to a causative agent of disease. It includes, without limitation, viruses, bacteria, fungi and parasites.

The term “pathogenesis” as used herein refers to the pathologic, physiologic or biochemical mechanism resulting in the development of a disease.

The term “pharmaceutical composition” is used herein to refer to a composition that is employed to prevent, reduce in intensity, cure or otherwise treat a target condition or disease. The terms “formulation” and “composition” are used interchangeably herein to refer to a product of the described invention that comprises all active and inert ingredients.

The term “pharmaceutically acceptable,” is used to refer to the carrier, diluent or excipient being compatible with the other ingredients of the formulation or composition and not deleterious to the recipient thereof. The carrier must be of sufficiently high purity and of sufficiently low toxicity to render it suitable for administration to the subject being treated. The carrier further should maintain the stability and bioavailability of an active agent. For example, the term “pharmaceutically acceptable” can mean approved by a regulatory agency of the Federal or a state government or listed in the U.S. Pharmacopeia or other generally recognized pharmacopeia for use in animals, and more particularly in humans.

“Phosphopinositide 3-Kinase Pathway, AKT/mTOR and PAK2/c-Abl”. The phosphoatidylinositol 3-kinase (PI3K) pathway is a non-Smad pathway contributing to TGF-β induced fibrosis. It induces two profibrotic pathways: Akt-mammalian target of rapamycin (mTOR) and p21-activated kinase 2 (PAK2)/Abelson kinase (c-Abl). [He, W and Dai, C. Curr. Pathobiol. Rep. (2015) 3: 183-92, citing Biernacka, A, et al. Growth Factors (2011) 29: 196-202; Tsou, P S et a. Am. J. Physiol. Cell Physiol. (2014) 307: C2-C13; Kamato, D. et al. Cell Signal (2013) 25: 2017-24; Wilkes, M C et al. Cancer Res. (2005) 65: 10431-40]. The phosphatidylinositol-3-kinase (PI3K)/Akt and the mammalian target of rapamycin (mTor) signaling pathways are crucial to many aspects of cell growth and survival. [Porta, C. et al., “Targeting PI2K/Akt/mTor signaling in cancer. Frontiers in Oncology (2014) doi.10.3389/fpmc.2014.00064). They are so interconnected that they could be regarded as a single pathway that, in turn, heavily interacts with many other pathways, including that of hypoxia inducible factors (HIFs).

PI3Ks constitute a lipid kinase family characterized by the capability to phosphorylate inositol ring 3′—OH group in inositol phospholipids. (Id., citing Fruman, D A et al., Phosphoinositide kinases. Annu. Rev. Biochem. (1998) 67: 481-507). Class I PI3Ks are heterodimers composed of a catalytic (CAT) subunit (i.e., p110) and an adaptor/regulatory subunit (i.e., p85). This classis further divided into two subclasses: subclass IA (PI3Kα, β, and δ), which is activated by receptors with protein tyrosine kinase activity, and subclass IB (PI3Kγ), which is activated by receptors coupled with G proteins (Id., citing Fruman, D A et al., Phosphoinositide kinases. Annu. Rev. Biochem. (1998) 67: 481-507).

Activation of growth factor receptor protein tyrosine kinases results in autophosphorylation on tyrosine residues. PI3K is then recruited to the membrane by directly binding to phosphotyrosine consensus residues of growth factor receptors or adaptors through one of the two SH2 domains In the adaptor subunit. This leads to allosteric activation of the CAT subunit. PI3K activation leads to the production of the second messenger phosphatidylinositol-4,4-bisphosphate (PI3,4,5-P3) from the substrate phosphatidylinositol-4,4-bisphosphate (PI-4,5-P2). PI3,4,5-P3 then recruits a subset of signaling proteins with pleckstrin homology (PH) domains to the membrane, including protein serine/threonine kinase-3′-phosphoinositide-dependent kinase I (PDK1) and Akt/protein kinase B (PKB) (Id., citing Fruman, D A et al., Phosphoinositide kinases. Annu. Rev. Biochem. (1998) 67: 481-507, Fresno-Vara, J A, et al., PI3K/Akt signaling pathway and cancer. Cancer Treat. Rev. (2004) 30: 193-204). Akt/PKB, on its own, regulates several cell processes involved in cell survival and cell cycle progression.

Akt. Akt (also known as protein kinase B) is a 60 kDa serine/threonine kinase. It is activated in response to stimulation of tyrosine kinase receptors such as platelet-derived growth factor (PDGF), insulin-like growth factor, and nerve growth factor (Shimamura, H, et al., J. Am. Soc. Nephrol. 14: 1427-1434, 2003; Datta K, Franke T F, Chan T O, Makris A, Yang S I, Kaplan D R, Morrison D K, Golemis E A, Tsichlis P N, Mol Cell Biol 15: 2304-2310, 1995; Kulik G, Klippel A, Weber M J, Mol Cell Biol 17: 1595-1606, 1997; Yao R, Cooper G M, Science 267: 2003-2006, 1995). Stimulation of Akt has been shown to be dependent on phosphatidylinositol 3-kinase (PI3-kinase) activity (Fruman D A, Meyers R E, Cantley L C, Annu Rev Biochem 67: 481-507, 1998; Choudhury G G, Karamitsos C, Hernandez J, Gentilini A, Bardgette J, Abboud H E, Am J Physiol 273: F931-938, 1997, Franke T F, Yang S I, Chan T O, Datta K, Kazlauskas A, Morrison D K, Kaplan D R, Tsichlis P N, Cell 81: 727-736, 1995; Franke T F, Kaplan D R, Cantley L C, Cell 88: 435-437, 1997).

Akt has been shown to act as a mediator of survival signals that protect cells from apoptosis in multiple cell lines (Brunet A, Bonni A, Zigmond M J, Lin M Z, Juo P, Hu L S, Anderson M J, Arden K C, Blenis J, Greenberg M E, Cell 96: 857-868, 1999; Downward J, Curr Opin Cell Biol 10: 262-267, 1998). For example, phosphorylation of the pro-apoptotic Bad protein by Akt was found to decrease apoptosis by preventing Bad from binding to the anti-apoptotic protein Bcl-XL (Dudek H, Datta S R, Franke T F, Birnbaum M J, Yao R, Cooper G M, Segal R A, Kaplan D R, Greenberg M E, Science 275: 661-665, 1997; Datta S R, Dudek H, Tao X, Masters S, Fu H, Gotoh Y, Greenberg M E, Cell 91: 231-241, 1997). Akt was also shown to promote cell survival by activating nuclear factor-kB (NF-kB) (Cardone M H, Roy N, Stennicke H R, Salvesen G S, Franke T F, Stanbridge E, Frisch S, Reed J C, Science 282: 1318-1321, 1998; Khwaja A, Nature 401: 33-34, 1999) and inhibiting the activity of the cell death protease caspase-9 (Kennedy S G, Kandel E S, Cross T K, Hay N, Mol Cell Biol 19: 5800-5810, 1999).

mTOR signaling pathway: Mechanistic target of rapamycin (mTOR) is an atypical serine/threonine kinase that is present in two distinct complexes. The first, mTOR complex 1 (mTORC1), is composed of mTOR, Raptor, GOL, and DEPTOR and is inhibited by rapamycin. It is a master growth regulator that senses and integrates diverse nutritional and environmental cues, including growth factors, energy levels, cellular stress, and amino acids. It couples these signals to the promotion of cellular growth by phosphorylating substrates that potentiate anabolic processes such as mRNA translation and lipid synthesis, or limit catabolic processes such as autophagy. The small GTPase Rheb, in its GTP-bound state, is a necessary and potent stimulator of mTORC1 kinase activity, which is negatively regulated by its GTPase-activating protein (GAP), the tuberous sclerosis heterodimer TSC1/2. TSC1 and TSC2 are the tumour-suppressor genes mutated in the tumour syndrome TSC (tuberous sclerosis complex). Their gene products form a complex (the TSC1-TSC2 (hamartin-tuberin) complex), which, through its GAP activity towards the small G-protein Rheb (Ras homologue enriched in brain), is a critical negative regulator of mTORC1 (mammalian target of rapamycin complex 1). (Huang, J. Manning B D, Biochem J. (2008) 412(2): 179-90). Most upstream inputs are funneled through Akt and TSC1/2 to regulate the nucleotide-loading state of Rheb. In contrast, amino acids signal to mTORC1 independently of the PI3K/Akt axis to promote the translocation of mTORC1 to the lysosomal surface where it can become activated upon contact with Rheb. This process is mediated by the coordinated actions of multiple complexes, including the v-ATPase, Ragulator, the Rag GTPases, and GATOR1/2. The second complex, mTOR complex 2 (mTORC2), is composed of mTOR, Rictor, GOL, Sin1, PRR5/Protor-1, and DEPTOR. mTORC2 promotes cellular survival by activating Akt, regulates cytoskeletal dynamics by activating PKCa, and controls ion transport and growth via SGK1 phosphorylation. Aberrant mTOR signaling is involved in many disease states

PI3K also acts as a branch point in response to TGF-β, leading to activation of PAK2/c-Abl, which stimulates collagen gene expression in normal fibroblasts, and induces fibroblast proliferation, thereby increasing the number of myofibroblast precursors. [[He, W and Dai, C. Curr. Pathobiol. Rep. (2015) 3: 183-92 citing Wilkes, M C and Leof, EB. J. Biol. Chem. (2006) 281: 27846-54]. PAK2/c-Abl promotes fibrosis through its downstream mediators, including PKCδ/Fli-1 and early growth response (Egr)-1, -2, and -3 {Id., citing Tsou, P S et al. A. J. Physiol. Cell Physiol. (2014) 307: C2-C13; Bhattacharyya, S. et al. J. Pathol. (2013) 229: 286-97; Fang, F. et al. Am. J. Pathol. (2013) 183: 1197-1208}.

The term “Plasma-Lyte” or Plasma-Lyte 148” as used herein refers to an isotonic, buffered intravenous crystalloid solution with a physiochemical composition that closely reflects human plasma.

The term “potency” and its various grammatical forms as used herein, refers to power or strength of a formulation.

The term “pulmonary compliance” as used herein refers to the change in lung volume per unit change in pressure. Dynamic compliance is the volume change divided by the peak inspiratory transthoracic pressure. Static compliance is the volume change divided by the plateau inspiratory pressure. Pulmonary compliance measurements reflect the elastic properties of the lungs and thorax and are influenced by factors such as degree of muscular tension, degree of interstitial lung water, degree of pulmonary fibrosis, degree of lung inflation, and alveolar surface tension (Doyle D J, O'Grady K F. Physics and Modeling of the Airway, D, in Benumof and Hagberg's Airway Management, 2013). Total respiratory system compliance is given by the following calculation:

C=ΔV/ΔP

where ΔV=change in lung volume, and ΔP=change in airway pressure This total compliance may be related to lung compliance and thoracic (chest wall) compliance by the following relation:

$$\frac{1}{{C}_{T}} = \frac{1}{{C}_{L}} + \frac{1}{{C}_{Th}}$$

where CT=total compliance (e.g., 100 mL/cm H₂O)
CL=lung compliance (e.g., 200 mL/cm H₂O)
CTh=thoracic compliance (e.g., 200 mL/cm H₂O)
   The values shown in parentheses are some typical normal adult values that can be used for modeling purposes (Id.).

It has been reported that soon after onset of respiratory distress from COVID, patients initially retain relatively good compliance despite very poor oxygenation. [Marini, J J and Gattinoni, L., JAMA Insights (2020) doi: 10.1001/jama.2020.6825, citing Grasselli, G. et al., JAMA (2020) doi: 10.1001/jama.2020.5394; Arentz, M. et al. JAMA (2020) doi: 10.1001/jama.2020.4326]. Minute ventilation is characteristically high. Infiltrates are often limited in extent and, initially, are usually characterized by a ground-glass pattern on CT that signifies interstitial rather than alveolar edema. Many patients do not appear overtly dyspneic. These patients can be assigned, in a simplified model, to “type L,” characterized by low lung elastance (high compliance), lower lung weight as estimated by CT scan, and low response to PEEP. {Id., citing Gattinoni, L. et al. Intensive Care Med. (2020) doi: 10.1007/s00134-020-06033-2}. For many patients, the disease may stabilize at this stage without deterioration while others, either because of disease severity and host response or suboptimal management, may transition to a clinical picture more characteristic of typical ARDS. These can be defined as “type H,” with extensive CT consolidations, high elastance (low compliance), higher lung weight, and high PEEP response. Types L and H are the conceptual extremes of a spectrum that includes intermediate stages, in which their characteristics may overlap.

The term “potency” as used herein and its various grammatical forms is an expression of the activity of a drug in terms of the concentration or amount of the drug required to produce a defined effect.

The term “precision medicine” as used herein refers to an approach for disease treatment and prevention that takes into account individual variability in genes, environment and lifestyle. A precision medicine approach allows for a more accurate prediction of which treatment and prevention strategies for a particular disease will work in which groups of patients. This is in contrast to a one-size-fits-all approach, in which disease treatment and prevention strategies are developed for the average person with less consideration for differences between individuals.

The term “progression” as used herein refers in medicine to the course of a disease as it becomes worse or spreads in the body.

The term “purification” and its various grammatical forms as used herein refers to the process of isolating or freeing from foreign, extraneous, or objectionable elements.

The term “reactive oxygen species” as used herein refers to oxygen-containing reactive species. It is a collective term to include superoxide (O2.—), hydrogen peroxide (H2O2), hydroxyl radical (OH.), singlet oxygen (1O2), peroxyl radical (LOO.), alkoxyl radical (LO.), lipid hydroperoxide (LOOH), peroxynitrite (ONOO—), hypochlorous acid (HOCl), and ozone (O3), among others. [Li, R. et al. React. Oxyg. Species (Apex) (2016) 1 (1): 9-21]

The term “recombinant” as used herein refers to an artificial combination of two otherwise separated segments of sequence, e.g., by chemical synthesis or by the manipulation of isolated segments of nucleic acids by genetic engineering techniques.

The term “rejuvenate” and its various grammatical forms as used herein refer to making young or youthful again. The term “resuscitate” as used herein refers to being restored to life or being revived.

The term “RISC” or RNA-induced silencing complex, as used herein, refers to a multiprotein complex that incorporates one strand of a small interfering RNA (siRNA) or micro RNA (miRNA). RISC uses the siRNA or miRNA as a template for recognizing complementary mRNA. When it finds a complementary strand, it activates RNase and cleaves the RNA. This process is important both in gene regulation by microRNAs and in defense against viral infections, which often use double-stranded RNA as an infectious vector.

Redox signaling refers to a physiological process, where ROS act as second messengers to mediate responses that are required for proper function and survival of the cell. On the other hand, redox modulation (or redox regulation) refers to a process wherein ROS alter the activity or function of the redox-sensitive molecular targets, including signaling proteins and metabolic enzymes, leading to either physiological or pathophysiological responses. When pathophysiological responses occur it is also known as oxidative stress. [Li, R. et al. React. Oxyg. Species (Apex) (2016) 1 (1): 9-21].

The term “repair” as used herein as a noun refers to any correction, reinforcement, reconditioning, remedy, making up for, making sound, renewal, mending, patching, or the like that restores function. When used as a verb, it means to correct, to reinforce, to recondition, to remedy, to make up for, to make sound, to renew, to mend, to patch or to otherwise restore function.

The term “reverse” as used herein refers to turning backward or in an opposite direction.

The terms “staging” or “clinical staging” are used interchangeably herein to refer to a process of determining details about the extent of a disease that guide decisions about treatment.

The term “signal transducers and activators of transcription” or “STATS” refers to a family of seven transcription factors activated by many cytokine and growth factor receptors. There are seven STATs (1-4, 5a, 5b, and 6), which reside in the cytoplasm in an inactive form until activated by cytokine receptors. Before activation, most STATS form homodimers, due to a specific homotypic interaction between domains present at the amino termini of the individual STAT proteins. The receptor specificity of each STAT is determined by the recognition of the distinctive phosphotyrosine sequence on each activated receptor by the different SH2 domains within the various STAT proteins. Recruitment of a STAT to the activated receptor brings the STAT close to an activated Janus kinase (JAK), which can then phosphorylate a conserved tyrosine residue in the carboxy terminus of the particular STAT. This leads to a rearrangement, in which the phosphotyrosine of each STAT protein binds to the SH2 domain of the other STAT, forming a configuration that can bind DNA with high affinity. Activated STATS predominantly form homodimers, with cytokine typically activating one type of STAT. For example, IFN-gamma activates STAT1 and generates STAT 1 homodimers, while IL-4 activates STAT6, generating STAT1 homodimers. Other cytokine receptors can activate several STATS, and some STAT heterodimers can be formed. The phosphorylated STAT dimer enters the nucleus, where it acts as a transcription factor to initiate the expression of selected genes that can regulate growth and differentiation of particular subsets of lymphocytes. [Janeway's Immunobiology, 9^{th} Ed., Murphy K. & Weaver, C. Eds. Garland Science, New York (2017) at 110-111].

The term “signature” as used herein refers to a specific and complex combination of biomarkers that reflect a biological state.

The term “Sirt1” as used herein refers to a member of the sirtuin family. Sirtuins are evolutionarily conserved proteins that use nicotinamide adenine dinucleotide (NAD+) as a co-substrate in their enzymatic reactions. There are seven proteins (SIRT1-7) in the human sirtuin family, among which SIRT1 is the most conserved and characterized. Sirt1 is a nicotinamide adenosine dinucleotide (NAD)-dependent deacetylase that removes acetyl groups from several transcription factors and regulatory proteins that are involved in inflammation, antioxidant expression, DNA repair, mitochondrial function, proteostasis, including autophagy. It inhibits cellular senescence and PI3K-mTOR signaling and restores defective autophagy. More specifically, Sirt1 activates FOXo3a, which regulates antioxidants (superoxide dismutases and catalase), activates PGC-1α, a transcription factor that maintains mitochondrial function, inhibits p53 induced senescence, and inhibits NF-κB thereby suppressing the senescence-associated secretory phenotype (SASP). The SASP response is activated by p2^{CIP1}, which results in activation of p38 mitogen activated protein (MAP) kinase and Janus-activated kinases (JAK), which results in the activation of NF-κB and secretion of proinflammatory cytokines (e.g., IL-1β, IL-6, TNFα), growth factors (e.g., VEGF, TGF-β), chemokines (e.g., CXCL1, CXCL8, CCL2) and matrix metalloproteinases (e.g., MMP-2, MMP-9), which are all increased in age-related diseases, including COPD. [Barnes, P J et al. Am L. Respir. Crit. Care Med. (2019) 200 (5): 556-64]. Plasminogen activator inhibitor-1 (PAI-1), another characteristic SASP protein, is increased in the sputum, sputum macrophages, and alveoli of patients with COPD [Id., citing To, M. et al. Chest (2013) 144: 515-21] and in IPF (Id., citing Schlliga, M. et al. Int. J. Biochem. Cell Biol. (2018) 97: 108-117).

Sirt1 has been implicated in a broad range of physiological functions, including control of gene expression, metabolism and aging [Rahman, S. and Islam, R. Cell Communication & Signaling (2011) article 11, citing Michan, S. & Sinclair, D. Biochem. J (2007) 404: 1-13; Haigis, M C and Guarente, LP. Genes Dev. (2006) 20: 2913-21; Yamamoto, H. et al. Mol. Endocrinol. (2007) 21: 1745-55]. Whereas an increase in the expression of the SIRT1 protein has been observed in cancer [Elibol, B. and Kilic, U. Front. Endocrinol. (Laussane) (2018) 9: 614, citing Chen, W Y et al. Cell (2005) 123: 437-48; Wang, C. et al. Nat. Cell Biol. (2006) 8: 1025-31], reductions in the SIRT1 level are more common in other diseases such as Alzheimer's Diseases (AD), Parkinson Disease (PD), obesity, diabetes, and cardiovascular diseases [Id., citing Lutz, M I et al. Neuromol. Med. (2014) 16: 405-14; Costa dos Santos, C. et al. Obes. Surg. (2010) 20: 633-9; Singh, P. et al. BMC Neurosci. (2017) 18: 46; Chan, S H et al. Redox Biol. (2017) 13: 301-9; Aditya, R. et al. Curr. Phar. Des. (2017) 23: 2299-307]. Recent developments elucidated the relation between downregulation of SIRT1 levels and disease progression as an increase in oxidative stress and inflammation [Id., citing Singh, P. et al. BMC Neurosci. (2017) 18: 46; Chan, S H et al. Redox Biol. (2017) 13: 301-9]. The list of Sirt1 substrates is continuously growing and includes several transcription factors: the tumor suppressor protein p53, members of the FoxO family (forkhead box factors regulated by insulin/Akt), HES1 (hairy and enhancer of split 1), HEY2 (hairy/enhancer-of-split related with YRPW motif 2), PPARγ (peroxisome proliferator-activated receptor gamma), CTIP2 [chicken ovalbumin upstream promoter transcription factor (COUPTF)-interacting protein 2], p300, PGC-1α (PPARγ coactivator), and NF-κB (nuclear factor kappa B) [Rahman, S. and Islam, R. Cell Communication & Signaling (2011) article 11, citing Michan S. and Sinclair D. Biochem. J. (2007) 404: 1-13; Haigis, M C and Guarente, LP. Genes Dev. (2006) 20: 2913-21; Yamamoto, H. et al. Mol. Endocrinol. (2007) 21: 1745-55].

The term “skeletal muscle satellite cells” as used herein refers to myogenic stem cells residing between the myofiber plasmalemma and basal lamina that can self-renew and produce differentiated progeny. Skeletal muscle satellite cells may be identified by the specific expression of the paired box transcription factor Pax-7. [Yablonka-Reuveni, Z. J. Histochem. Cytochem. (2011) 59 (12): 1041-59].

The term “slow” as used herein refers to holding back progress or development.

The terms “soluble” and “solubility” refer to the property of being susceptible to being dissolved in a specified fluid (solvent). The term “insoluble” refers to the property of a material that has minimal or limited solubility in a specified solvent. In a solution, the molecules of the solute (or dissolved substance) are uniformly distributed among those of the solvent. Among the acceptable vehicles and solvents that may be employed are water, Ringer's solution, and isotonic sodium chloride solution.

A “solution” generally is considered as a homogeneous mixture of two or more substances. It is frequently, though not necessarily, a liquid. In a solution, the molecules of the solute (or dissolved substance) are uniformly distributed among those of the solvent.

The term “solvate” as used herein refers to a complex formed by the attachment of solvent molecules to that of a solute.

The term “solvent” as used herein refers to a substance capable of dissolving another substance (termed a “solute”) to form a uniformly dispersed mixture (solution).

The term “stem cells” refers to undifferentiated cells having high proliferative potential with the ability to self-renew that can generate daughter cells that can undergo terminal differentiation into more than one distinct cell phenotype. The term “renewal” or “self renewal” as used herein, refers to the process by which a stem cell divides to generate one (asymmetric division) or two (symmetric division) daughter cells having development potential indistinguishable from the mother cell. Self-renewal involves both proliferation and the maintenance of an undifferentiated state.

The term “adult (somatic) stem cells” as used herein refers to undifferentiated cells found among differentiated cells in a tissue or organ. Their primary role in vivo is to maintain and repair the tissue in which they are found. Adult stem cells, which have been identified in many organs and tissues, including brain, bone marrow, peripheral blood, blood vessels, skeletal muscles, skin, teeth, gastrointestinal tract, liver, ovarian epithelium, and testis, are thought to reside in a specific area of each tissue, known as a stem cell niche, where they may remain quiescent (non-dividing) for long periods of time until they are activated by a normal need for more cells to maintain tissue, or by disease or tissue injury. Mesenchymal stem cells are an example of adult stem cells.

As used herein, the phrase “subject in need” of treatment for a particular condition is a subject having that condition, diagnosed as having that condition, or at risk of developing that condition. According to some embodiments, the phrase “subject in need” of such treatment also is used to refer to a patient who (i) will be administered a composition of the described invention; (ii) is receiving a composition of the described invention; or (iii) has received at least one a composition of the described invention, unless the context and usage of the phrase indicates otherwise.

The terms “surfactant protein A (SP-A)” and “surfactant protein D (SP-D)” refer to hydrophobic, collagen-containing calcium-dependent lectins, with a range of nonspecific immune functions at pulmonary and cardiopulmonary sites. SP-A and SP-D play crucial roles in the pulmonary immune response, and are secreted by type II pneumocytes, nonciliated bronchiolar cells, submucosal glands, and epithelial cells of other respiratory tissues, including the trachea and bronchi. SP-D is important in maintaining pulmonary surface tension, and is involved in the organization, stability, and metabolism of lung parenchyma (Wang K, et al. Medicine (2017) 96 (23): e7083). An increase of 49 ng/mL (1 SD) in baseline SP-A level was associated with a 3.3-fold increased risk of mortality in the first year after presentation. SP-A and SP-D are predictors of worse survival in a one year mortality regression model (Guiot, J. et al. Lung (2017) 195(3): 273-280).

The term “suspension” as used herein refers to a dispersion (mixture) in which a finely-divided species is combined with another species, with the former being so finely divided and mixed that it doesn't rapidly settle out. In everyday life, the most common suspensions are those of solids in liquid.

The term “symptom” as used herein refers to a sign or an indication of disorder or disease, especially when experienced by an individual as a change from normal function, sensation, or appearance.

As used herein, the term “therapeutic agent” or “active agent” refers to refers to the ingredient, component or constituent of the compositions of the described invention responsible for the intended therapeutic effect.

The term “therapeutic component” as used herein refers to a therapeutically effective dosage (i.e., dose and frequency of administration) that eliminates, reduces, or prevents the progression of a particular disease manifestation in a percentage of a population. An example of a commonly used therapeutic component is the ED50, which describes the dose in a particular dosage that is therapeutically effective for a particular disease manifestation in 50% of a population.

The term “therapeutic effect” as used herein refers to a consequence of treatment, the results of which are judged to be desirable and beneficial. A therapeutic effect may include, directly or indirectly, the arrest, reduction, or elimination of a disease manifestation. A therapeutic effect may also include, directly or indirectly, the arrest, reduction, or elimination of the progression of a disease manifestation.

The term “therapeutic signature” as used herein refers to a specific and complex combination of biomarkers that reflect a biological state that leads to a specific therapeutic effect.

As used herein, the term “tissue” refers to a collection of similar cells and the intercellular substances surrounding them. For example, adipose tissue is a connective tissue consisting chiefly of fat cells surrounded by reticular fibers and arranged in lobular groups or along the course of smaller blood vessels. Connective tissue is the supporting or framework tissue of the body formed of fibrous and ground substance with numerous cells of various kinds. It is derived from the mesenchyme, and this in turn from the mesoderm. The varieties of connective tissue include, without limitation, areolar or loose; adipose; sense, regular or irregular, white fibrous; elastic; mucous; lymphoid tissue; cartilage and bone.

The term “tissue inhibitors of metalloproteases” or “TIMPs” as used herein refers to key regulators of the metalloproteinases that degrade the extracellular matrix and shed cell surface molecules. [Brew, K. and Nagase, H. Biochim. Biophys. Acta (2010) 1803 (1): 55-71]. TIMPs can undergo changes in molecular dynamics induced by their interactions with proteases. TIMPs also have biological activities that are independent of metalloproteinases; these include effects on cell growth and differentiation, cell migration, anti-angiogenesis, anti- and pro-apoptosis, and synaptic plasticity. The human genome has 4 paralogous genes encoding TIMPs (TIMPs-1 to -4). All four TIMPs inhibit MMPs, but with affinities that vary for different inhibitor-protease pairs. The four human TIMPs are, in general terms, broad-spectrum inhibitors of the 23 MMPs found in humans, but there are some differences in specificity among them. TIMP-1 is more restricted in its inhibitory range than the other three TIMPs, having a relatively low affinity for the membrane-type MMPs, MMP-14, MMP-16, and MMP-24 as well as for MMP-19. There are some relatively subtle differences between the affinities of different TIMPs for other MMPs. For example, TIMPs-2 and -3 are weaker inhibitors than TIMP-1 for MMP-3 and MMP-7, contrasting with their affinities for other MMPs [Id., citing Hamze, Abe et al. Protein Sci. (2007) 16: 1905-13]]. TIMP-3 is unique among the mammalian TIMPs in inhibiting a broader array of metalloproteinases including several members of the aggrecanase ADAM and ADAMTS families [Id., citing Amour, A. et al. FEBS Lett. (1998) 435: 39-44; Kashiwagi, M. et al. J. Biol. Chem. (2001) 276: 12501-4; Amour, A. et al. FEBS Lett. (2000) 473: 275-9; Hashimoto, G. et al. FEBS Lett. (2001) 494: 192-5; Wang, W M et al. Biochem. J. (2006) 398: 515-19; Jacobssen, J. et al. Biochemistry (2008) 47: 537-47]. Other TIMPs have limited inhibitory activities for ADAMs: TIP-1 and TIMP-2 inhibit ADAM10 [Id., citing Amour, A. et al. FEBS Lett. (2000) 473: 275-9] and ADAM12 [Id., citing Jacobsen, J. et al. Biochemistry (2008) 47: 537-47], respectively. TIP-3 and N-TIMP-4, but not full-length TIMP-4, inhibit ADAM17 [Id., citing Lee, M H et al. J. Biol. Chem. (2005) 280: 15967-75]. TIMP-4 was also reported to inhibit ADAM28 [Mochizuki, S. et al. Biochem. Biophys. Res. Commun. (2004) 315: 79-84]. ADAM metalloproteinases differ from the MMPs in domain structures and are highly divergent in catalytic domain sequences: ADAMs are membrane-bound enzymes containing disintegrin, cysteine-rich, EGF-like and transmembrane domains C-terminal to their catalytic domains [Id., citing Edwards, D R et al. Mol. Aspects Med. (2008) 29: 258-89]; and ADAMTS (disintegrin-metalloproteinases with thrombospondin motifs) are secreted proteins with a disintegrin domain and variable numbers of thrombospondin type 1 motifs and other domains in their C-terminal regions [Id., citing Porter, S. et al. Biochem. J. (2005) 386: 15-27]. The term “toll-like receptor” or “TLRs” as used herein refers to innate receptors on macrophages, dendritic cells and some other cells that recognize pathogens and their products, such as bacterial lipopolysaccharide (LPS). Recognition stimulates the receptor-bearing cells to produce cytokines that help initiate immune responses. For example, TLR-1 is a cell surface toll-like receptor that acts in a heterodimer with TLR-2 to recognize lipoteichoic acid and bacterial lipoproteins. TLR-2 is a cell surface toll-like receptor that acts in a heterodimer with either TLR-1 or TLR-6 to recognize lipoteichoic acid and bacterial lipoproteins. TLR-4 is a cell surface toll-like receptor that, in conjunction with accessory proteins MD-2 and CD14, recognizes bacterial lipopolysaccharide and lipoteichoic acid. TLR5 is a cell surface toll-like receptor that recognizes the flagellin protein of bacterial flagella. TLR 6 is a cell surface toll-like receptor that acts in a heterodimer with TLR2 to recognize lipoteichoic acid and bacterial lipoproteins. TLR3 is an endosomal toll-like receptor that recognizes double-stranded viral RNA. TLR-7 is an endosomal toll-like receptor that recognizes single-stranded viral RNA. TLR-8 is an endosomal toll-like receptor that recognizes single-stranded viral RNA. TLR-9 is an endosomal toll-like receptor that recognizes DNA containing unmethylated CpG.

Smad-dependent pathway for TGF-β signaling. The classical Smad-dependent pathway for transforming growth factor-β (TGFβ) signaling occurs when TGF-β receptor type 2, which is constitutively active, transphosphorylates and forms a complex with the TGF-β-bound TGF-β receptor type 1. This complex then phosphorylates serine residues of cytoplasmic receptor-activated Smad (R-Smad), a complex of Smad2 and Smad3. These two heterodimerize and bind to the common mediator Smad (Co-Smad) Smad4, and the whole complex translocates across the nuclear membrane to interact with specific cis-acting elements in the regulatory regions of its target genes [(He, W. and Dai, C. Curr. Pathobiol. Rep. (2015) 3(2): 183-92), citing Tsou, P S et al. Am. J. Physiol. Cell Physiol. (2014) 307: C2-13], recruiting coactivators such as p300 and CBP; corepressors such as c-Ski, SnoN, transforming growth-inhibiting factor, and Smad nuclear-interacting protein 1; or transcription factors such as AP-1 and Sp1 to modulate gene expression [Id., citing Biernacka, A. et al. Growth Factors (2011) 29: 196-202]. Inhibitory Smad (I-Smad) Smad6 or Smad7, acting as negative regulators, not only antagonizes the TGF-β/Smad pathway by binding to TGF-β1 or competing with activated R-Smad for binding to Co-Smad, but also recruits the E3 ubiquitin-protein ligases Smurf1 and Smurf2, which target Smad proteins for proteasomal degradation, thereby blocking Smad2/3 activation, facilitating receptor degradation, and eventually terminating Smad-mediated signaling.

The term “transcriptome” as used herein refers to the full range of messenger RNA (or mRNA) molecules expressed by an organism. The term “transcriptome” also refers to the array of mRNA transcripts produced in a particular cell or tissue type.

The terms “treat,” “treated,” or “treating” as used herein refers to both therapeutic treatment and/or prophylactic or preventative measures, wherein the object is to prevent or slow down (lessen) an undesired physiological condition, disorder or disease, or to obtain beneficial or desired clinical results. For the purposes of this invention, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment.

The term “tumor necrosis factor receptor-associated factor 6” or TRAF6” as used herein refers to an E3 ligase that produces a K63 polyubiquitin signaling scaffold in TLR-4 signaling to activate the NFκB pathway.

The term “tumor susceptibility gene 101 or Tsg101” as used herein refers to a housekeeping gene highly conserved between mouse and human for which significant variations in high or low protein expression levels in normal tissues or cancer cells are likely a consequence of post-transcriptional or post-translational mechanisms. It has been suggested to function as a negative regulator of ubiquitin-mediated protein degradation [Ferraiuolo, R-M, et al., Cancers (Basel) (2020) 12 (20: 450, citing Koonin, E V and Abagyan, RA. Nat. Genet. (1997) 75: 467-69) as well as a mediator for the intracellular movement of ubiquinated proteins [Id., citing Katzmann, D J et al. Cell (2001) 106: 145-55].

The terms “usual interstitial pneumonia” or “UIP” pattern are used interchangeably herein to refer to a morphologic entity defined by a combination of 1) patchy interstitial fibrosis with alternating areas of normal lung, (2) temporal heterogeneity of fibrosis characterized by scattered fibroblastic foci in the background of dense acellular collagen, and (3) architectural alteration due to chronic scarring or honeycomb change [Am. Thoracic Society (ATS)?European Respiratory Society (ERS), Am. J. Resp. Crit. Care Med. (2002) 165(2): 277-304. UIP is not entirely synonymous with IPF, and diagnosis of IPF requires an exclusion of possible underlying clinical conditions, including collagen vascular disease, drug toxicity, chronic hypersensitivity pneumonitis, asbestosis, familial IPF, and Hermansky-Pudlak syndrome. [Id.]

The term “WNT1 (Wnt Family Member 1)” as used herein refers to a protein coding gene and a member of the WNT gene family. Protein Wnt-1 is encoded by the WNT1 gene and is very conserved in evolution. It acts in the canonical Wnt signaling pathway by promoting beta-catenin-dependent transcriptional activation. Activation of Wnt/P catenin signaling has been reported in skin, kidney, liver, lung and cardiac fibrosis. [He, W and Dai, C. Curr. Pathobiol. Rep (2015) 3: 183-92, citing Wynn, T A and Ramalingam, TR. Nat. Med. (2012) 18: 1028-40; Lam, A P and Gottardi, CJ. Curr. Opin. Rheumatol. (2011) 23: 562-7]. Wnt proteins deliver their signal across the plasma membrane by interacting with Fizzled receptors and coreceptors. Once Wnts bind to their receptors/coreceptors, they initiate a chain of downstream signaling events leading to dephosphorylation of β-catenin [Id., citing Liu, Y. J. Am. Soc. Nephrol. (2010) 21: 212-22]. Escaping from degradation mediated by the ubiquitin/proteasome system stabilized β catenin accumulates in the cytoplasm and translocates into the nucleus, where it interacts with its DNA-binding partner known as T cell factor (TCF)/lymohpcyte enhancer-binding factor 1 (LEF1) to stimulate the transcription of Wnt target genes. [Id., citing Lam, A P and Gottardi, CJ. Curr. Opin. Rheumatol. (2011) 23: 562-67; Liu, Y. J. Am. Soc. Nephrol. (2010) 21: 212-22; Huang, H. & He, X. Curr. Opin. Cell Biol. (2008) 20: 119-25}.

The term “wound healing” refers to the process by which the body repairs trauma to any of its tissues, especially those caused by physical means and with interruption of continuity.

A wound-healing response often is described as having three distinct phases-injury, inflammation and repair. Generally speaking, the body responds to injury with an inflammatory response, which is crucial to maintaining the health and integrity of an organism. If, however, it goes awry, it can result in tissue destruction.

Although these three phases are often presented sequentially, during chronic or repeated injury, these processes function in parallel, placing significant demands on regulatory mechanisms. (Wilson and Wynn, Mucosal Immunol., 2009, 3(2): 103-121).

### Phase I: Injury

Injury caused by factors including, but not limited to, autoimmune or allergic reactions, environmental particulates, or infection or mechanical damage, often results in the disruption of normal tissue architecture, initiating a healing response. Damaged epithelial and endothelial cells must be replaced to maintain barrier function and integrity and prevent blood loss, respectively. Acute damage to endothelial cells leads to the release of inflammatory mediators and initiation of an anti-fibrinolytic coagulation cascade, temporarily plugging the damaged vessel with a platelet and fibrin-rich clot. For example, lung homogenates, epithelial cells or bronchoalveolar lavage fluid from idiopathic pulmonary fibrosis (IPF) patients contain greater levels of the platelet-differentiating factor, X-box-binding protein-1, compared with chronic obstructive pulmonary disease (COPD) and control patients, suggesting that clot-forming responses are continuously activated. In addition, thrombin (a serine protease required to convert fibrinogen into fibrin) is also readily detected within the lung and intra-alveolar spaces of several pulmonary fibrotic conditions, further confirming the activation of the clotting pathway. Thrombin also can directly activate fibroblasts, increasing proliferation and promoting fibroblast differentiation into collagen-producing myofibroblasts. Damage to the airway epithelium, specifically alveolar pneumocytes, can evoke a similar anti-fibrinolytic cascade and lead to interstitial edema, areas of acute inflammation, and separation of the epithelium from the basement membrane.

Platelet recruitment, degranulation and clot formation rapidly progress into a phase of vasoconstriction with increased permeability, allowing the extravasation (movement of white blood cells from the capillaries to the tissues surrounding them) and direct recruitment of leukocytes to the injured site. The basement membrane, which forms the extracellular matrix underlying the epithelium and endothelium of parenchymal tissue, precludes direct access to the damaged tissue. To disrupt this physical barrier, zinc-dependent endopeptidases, also called matrix metalloproteinases (MMPs), cleave one or more extracellular matrix constituents allowing extravasation of cells into, and out of, damaged sites.

### Phase H: Inflammation

Once access to the site of tissue damage has been achieved, chemokine gradients recruit inflammatory cells. Neutrophils, eosinophils, lymphocytes, and macrophages are observed at sites of acute injury with cell debris and areas of necrosis cleared by phagocytes.

The early recruitment of eosinophils, neutrophils, lymphocytes, and macrophages providing inflammatory cytokines and chemokines can contribute to local TGF-β and IL-13 accumulation. Following the initial insult and wave of inflammatory cells, a late-stage recruitment of inflammatory cells may assist in phagocytosis, in clearing cell debris, and in controlling excessive cellular proliferation, which together may contribute to normal healing. Late-stage inflammation may serve an anti-fibrotic role and may be required for successful resolution of wound-healing responses. For example, a late-phase inflammatory profile rich in phagocytic macrophages, assisting in fibroblast clearance, in addition to IL-10-secreting regulatory T cells, suppressing local chemokine production and TGF-β, may prevent excessive fibroblast activation.

The nature of the insult or causative agent often dictates the character of the ensuing inflammatory response. For example, exogenous stimuli like pathogen-associated molecular patterns (PAMPs) are recognized by pathogen recognition receptors, such as toll-like receptors and NOD-like receptors (cytoplasmic proteins that have a variety of functions in regulation of inflammatory and apoptotic responses), and influence the response of innate cells to invading pathogens. Endogenous danger signals also can influence local innate cells and orchestrate the inflammatory cascade.

The nature of the inflammatory response dramatically influences resident tissue cells and the ensuing inflammatory cells. Inflammatory cells themselves also propagate further inflammation through the secretion of chemokines, cytokines, and growth factors. Many cytokines are involved throughout a wound-healing and fibrotic response, with specific groups of genes activated in various conditions. Fibrotic lung disease (such as idiopathic pulmonary fibrosis) patients more frequently present pro-inflammatory cytokine profiles (including, but not limited to, interleukin-1 alpha (IL-1α), interleukin-1 beta (IL-1β), interleukin-6 (IL-6), tumor necrosis factor alpha (TNF-α), transforming growth factor beta (TGF-β), and platelet-derived growth factors (PDGFs)). Each of these cytokines has been shown to exhibit significant pro-fibrotic activity, acting through the recruitment, activation and proliferation of fibroblasts, macrophages, and myofibroblasts.

### Phase III: Tissue Repair and Contraction

The closing phase of wound healing consists of an orchestrated cellular reorganization guided by a fibrin (a fibrous protein that is polymerized to form a “mesh” that forms a clot over a wound site)-rich scaffold formation, wound contraction, closure and re-epithelialization. The vast majority of studies elucidating the processes involved in this phase of wound repair have come from dermal wound studies and in vitro systems.

Myofibroblast-derived collagens and smooth muscle actin (α-SMA) form the provisional extracellular matrix, with macrophage, platelet, and fibroblast-derived fibronectin forming a fibrin scaffold. Collectively, these structures are commonly referred to as granulation tissues. Primary fibroblasts or alveolar macrophages isolated from IPF patients produce significantly more fibronectin and α-SMA than control fibroblasts, indicative of a state of heightened fibroblast activation. It has been reported that IPF patients undergoing steroid treatment had similar elevated levels of macrophage-derived fibronectin as IPF patients without treatment. Thus, similar to steroid resistant IL-13-mediated myofibroblast differentiation, macrophage-derived fibronectin release also appears to be resistant to steroid treatment, providing another reason why steroid treatment may be ineffective. From animal models, fibronectin appears to be required for the development of pulmonary fibrosis, as mice with a specific deletion of an extra type III domain of fibronectin (EDA) developed significantly less fibrosis following bleomycin administration compared with their wild-type counterparts.

In addition to fibronectin, the provisional extracellular matrix consists of glycoproteins (such as PDGF), glycosaminoglycans (such as hyaluronic acid), proteoglycans and elastin. Growth factor and TGF-β-activated fibroblasts migrate along the extracellular matrix network and repair the wound. Within skin wounds, TGF-β also induces a contractile response, regulating the orientation of collagen fibers. Fibroblast to myofibroblast differentiation, as discussed above, also creates stress fibers and the neo-expression of α-SMA, both of which confer the high contractile activity within myofibroblasts. The attachment of myofibroblasts to the extracellular matrix at specialized sites called the “fibronexus” or “super mature focal adhesions” pull the wound together, reducing the size of the lesion during the contraction phase. The extent of extracellular matrix laid down and the quantity of activated myofibroblasts determines the amount of collagen deposition. To this end, the balance of matrix metalloproteinases (MMPs) to tissue inhibitor of metalloproteinases (TIMPs) and collagens to collagenases vary throughout the response, shifting from pro-synthesis and increased collagen deposition towards a controlled balance, with no net increase in collagen. For successful wound healing, this balance often occurs when fibroblasts undergo apoptosis, inflammation begins to subside, and granulation tissue recedes, leaving a collagen-rich lesion. The removal of inflammatory cells, and especially α-SMA-positive myofibroblasts, is essential to terminate collagen deposition. Interestingly, in IPF patients, the removal of fibroblasts can be delayed, with cells resistant to apoptotic signals, despite the observation of elevated levels of pro-apoptotic and FAS-signaling molecules.

Several studies also have observed increased rates of collagen-secreting fibroblast and epithelial cell apoptosis in IPF, suggesting that yet another balance requires monitoring of fibroblast apoptosis and fibroblast proliferation. From skin studies, re-epithelialization of the wound site re-establishes the barrier function and allows encapsulated cellular re-organization. Several in vitro and in vivo models, using human or rat epithelial cells grown over a collagen matrix, or tracheal wounds in vivo, have been used to identify significant stages of cell migration, proliferation, and cell spreading. Rapid and dynamic motility and proliferation, with epithelial restitution from the edges of the denuded area, occur within hours of the initial wound. In addition, sliding sheets of epithelial cells can migrate over the injured area assisting wound coverage. Several factors have been shown to regulate re-epithelialization, including serum-derived transforming growth factor alpha (TGF-α), and matrix metalloproteinase-7 (MMP-7) (which itself is regulated by TIMP-1).

Collectively, the degree of inflammation, angiogenesis, and amount of extracellular matrix deposition all contribute to ultimate development of a fibrotic lesion.

### EMBODIMENTS

### Method of Preparing a Purified, Enriched Population of Exosomes

According to one aspect, the present disclosure provides a method of preparing a purified, enriched population of exosomes derived from a biological sample, wherein the biological sample is derived from a subject with a chronic lung disease, the method comprising:
(a) collecting a biological sample from the subject with the lung disease and from a healthy control;
(b) centrifuging the biological sample at low speed to form a clarified supernatant;
(c) ultracentrifuging the clarified supernatant to pellet the purified enriched population of exosomes; and
(d) characterizing the purified enriched population of exosomes, wherein.
   (i) the population of exosomes expresses two or more exosome biomarker selected from the group consisting of CD9, CD63, CD81, or HSP70;
   (ii) size of the exosomes in the population of exosomes ranges from 30 μm to 150 μm, inclusive;
   (iii) the population of exosomes comprises a total protein of at least about 100-200 μg, inclusive;
   (iv) the population of exosomes comprise a total RNA content of at least about 100-200 ng, inclusive; and
   (v) the population of exosomes comprises a cargo comprising dysregulated expression of two or more microRNAs selected frommiR-199, miR Let-7a, miR Let-7b, mir-Let-7d, miR-10a, miR-21, miR-29a, miR-34, miR-101, miR-125, miR-145, miR-146a, miR-181a, miR-181b, miR-181c, miR-199, and miR-142.

According to some embodiments, the biological sample is a body fluid. According to some embodiments, the body fluid is blood, breast milk, saliva, urine, bile, pancreatic juice, cerebrospinal fluid, amniotic fluid, or a peritoneal fluid. According to some embodiments, the body fluid is serum or urine. According to some embodiments, the body fluid is serum. According to some embodiments, the body fluid is urine. According to some embodiments, the number of exosomes in the population of exosomes comprises at least 10E10 particles.

According to some embodiments, the biological sample is obtained from a mammal. According to some embodiments, the biological sample is obtained from a non-human mammal or a human subject. According to some embodiments, the human subject is over 50 years of age. According to some embodiments, the healthy control is age and sex matched to the subject.

An exemplary protocol for isolation of a population of exosomes from a biological sample is as follows. A 4° C. biological sample is centrifuged at 3,000×g for 20 min at room temperature in a swinging bucket rotor to remove large cells and debris. The clarified supernatant is collected and ultracentrifuged at 100,000×g for 2 hours, in a fixed angle rotor at 4° C., to pellet exosomes. The exosome pellet is resuspended in a minimum volume of DPBS (approximately 120 uL/ultracentrifugation tube). Exosomes are characterized using a Thermo NanoDrop spectrophotometer for protein determination and approximate RNA concentration by direct absorbance; exosomes were not lysed, stained, or RNA extracted prior to taking these measurements. Particle diameter and concentration is assessed by tunable resistive pulse sensing (TRPS; (qNano, Izon Science Ltd) using a NP150 nanopore membrane at a 47 mm stretch. The concentration of particles is standardized using multi-pressure calibration with carboxylated polystyrene beads of a defined size (nm diameter) and at a defined concentration (particles/mL).

According to some embodiments, size of the population of exosomes ranges from about 30 μm to about 150 μm, inclusive, in diameter, i.e., about 30 μm, about 35 μm, about 40 μm, about 45 μm, about 50 μm, about 55 μm, about 60 μm, about 65 μm, about 70 μm, about 75 μm, about 80 μm, about 85 μm, about 90 μm, about 95 μm, about 100 μm, about 105 μm, about 110 μm, about 120 μm, about 125 μm, about 130 μm, about 135 μm, about 140 μm, about 145 μm, or about 150 am, in diameter.

According to some embodiments, the population of exosomes comprises a total protein content of at least about 100-200 μg, inclusive, i.e., at least about 100 μg, at least about 105 μg, at least about 110 μg, at least about 115 μg, at least about 120 μg, at least about 125 μg, at least about 130 μg, at least about 135 μg, at least about 140 μg, at least about 145 μg, at least about 150 μg, at least about 155 μg, at least about 160 μg, at least about 165 μg, at least about 170 μg, at least about 175 μg, at least about 180 μg, at least about 185 μg, about 190 μg, at least about 195 μg, or at least about 200 μg.

For RNA isolation a Norgen Preserved Blood RNA Purification Kit I can be used according to manufacturer's directions. Briefly, for every 100 μl of exosome preparation, 300 μL of lysis solution is added. After a short vortex, 400 μL of 95-100% ethanol is added. 600 μl of the lysate containing ethanol is loaded onto the column and centrifuged for 1 minute at >3,500×g (˜6,000 RPM). The column is washed three times with wash solution and centrifuged each time for 1 minute (14,000×g). The column is spun for an additional two minutes in order to thoroughly dry the resin and the column contents eluded with 50 μl of Elution Solution by centrifuging for 2 minutes at 200×g (˜2,000 RPM), followed by 1 minute at 14,000×g. According to some embodiments, the population of exosomes comprises a total RNA content of from at least about 100 ng to at least about 200 ng, inclusive, i.e., at least about 100 ng, at least about 105 ng, at least about 110 ng, at least about 115 ng, at least about 120 ng, at least about 125 ng, at least about 130 ng, at least about 135 ng, at least about 140 ng, at least about 145 ng, at least about 150 ng, at least about 155 ng, at least about 160 ng, at least about 165 ng, at least about 170 ng, at least about 175 ng, at least about 180 ng, at least about 185 ng, at least about 190 ng, at least about 195 ng, or at least about 200 ng.

According to some embodiments, the miRNA expression is a signature of a fibrotic lung disease. According to some embodiments, dysregulated miRNA expression of miR-199 and miR-34a in the population of exosomes derived from the biological sample of the subject compared to a population of exosomes derived from a biological sample derived from a healthy control is fibrotic. According to some embodiments, dysregulated miRNA expression of miR-142 in the population of exosomes derived from the biological sample of the subject compared to a population of exosomes derived from a biological sample derived from a healthy control is antifibrotic. According to some embodiments, dysregulated expression of miR-199 in the population of exosomes derived from the biological sample of a subject diagnosed with IPF compared to a population of exosomes derived from a biological sample derived from a healthy control is increased compared to a population of exosomes derived from a healthy control. According to some embodiments, the dysregulated expression of one or more of let-7D, miR-29, or miR-181 in the population of exosomes derived from the biological sample of the subject diagnosed with IPF compared to a population of exosomes derived from a biological sample derived from a healthy control is decreased, compared to the population of exosomes derived from the healthy control. According to some embodiments, dysregulated expression of miR-199 in the population of exosomes derived from the biological sample of the subject diagnosed with IPF compared to a population of exosomes derived from a biological sample derived from a healthy control and the dysregulated expression of one or more of let-7D, miR-29, miR-142, miR-181 derived from the population of exosomes derived from a biological sample from the subject diagnosed with IPF is decreased, compared to the population of exosomes derived from the healthy control. According to some embodiments, reduced or absent microRNA Let-7 expression in the population of exosomes derived from the biological sample of the subject compared to a population of exosomes derived from a biological sample derived from a healthy control is associated with early stage fibrotic lung disease. According to some embodiments, microRNA Let-7 expression in the population of exosomes derived from the biological sample of the subject compared to a population of exosomes derived from a biological sample derived from a healthy control is associated with end-stage fibrotic lung disease.

Examples of methods for exosome product characterization analysis include, without limitation, nanoparticle size and concentration analysis; flow cytometry analysis of exosome specific protein markers (CD63, HSP70), and pro-angiogenic and pro-migratory potency. Other exemplary analytics include, without limitation:cytokine testing using a 9-panel human angiogenesis multiplex ELISA comprising: Ang-2, FGF, HGF, IL-8, PDGF, TIMP-1, TIMP-2, and VEGF at standard dilutions of 1:2, 1:20, 1:200 and 1:800; Mycoplasma testing by multiplex detection by polymerase chain reaction; sterility testing for media by membrane filtration; and metabolite and pH analysis on the NOVA Flex 2 for Gln, Glu, Gluc, Lac, NH₄+, Na+, K+, Ca++, pH, pCO₂, pO₂ and osmolality.

According to some embodiments, the purified enriched population of exosomes provides enhanced therapeutic targeting/precision; improved cell selectivity/tropism, and directed pharmacology at desired cellular or tissue sites. According to some embodiments, the purified enriched population of exosomes provides enhanced therapeutic targeting/precision. According to some embodiments, the purified enriched population of exosomes provides improved cell selectivity/tropism. According to some embodiments, the purified enriched population of exosomes provides directed pharmacology at desired cellular or tissue sites.

According to some embodiments, markers for fibrosis (ay-integrin, collagen type 1 mRNA, TGF-β mRNA expression, and other downstream fibrotic pathways (e.g., c-Jun, AKT expression and MMP-9 activity) can be stimulated in an ex vivo assay by the population of exosomes derived from the biological sample (e.g., urine or serum) from a subject diagnosed with the fibrotic lung disease compared to a healthy control. According to some embodiments, the ex vivo assay is a human lung punch assay. According to some embodiments, decreased wound closure by the population of exosomes derived from the biological sample (e.g., urine or serum) from a subject diagnosed with the fibrotic lung disease compared to a healthy control can be measured in an ex vivo wound healing assay

### Methods for Diagnosis, Prognosis, and Treatment

According to another aspect, the present disclosure provides a method for noninvasively diagnosing and staging a progressive chronic lung disease characterized by disease related lung dysfunction comprising:
(a) quantifying lung function of a subject with a progressive chronic lung disease characterized by disease-related lung dysfunction;
(b) determining a stage of the chronic lung disease by:
   (i) collecting a biological sample from the subject with the lung disease and from a healthy control;
   (ii) centrifuging the biological sample at low speed to form a clarified supernatant;
   (iii) ultracentrifuging the clarified supernatant to pellet the purified enriched population of exosomes; and
   (iv) characterizing the purified enriched population of exosomes, wherein.
      (1) the population of exosomes expresses two or more exosome biomarker selected from the group consisting of CD9, CD63, CD81, or HSP70;
      (2) size of the exosomes in the population of exosomes ranges from 30 μm to 150 μm, inclusive;
      (3) the population of exosomes comprises a total protein of at least about 100 μg-200 μg, inclusive;
      (4) the population of exosomes comprise a total RNA content of at least about 100 ng-200 ng, inclusive; and
      (5) the population of exosomes comprises a cargo comprising dysregulated expression of two or more microRNAs selected from miR-199, miR Let-7a, miR Let-7b, mir-Let-7d, miR-10a, miR-21, miR-29a, miR-34, miR-101, miR-125, miR-145, miR-146a, miR-181a, miR-181b, miR-181c, miR-199, and miR-142, and

wherein expression of the two or more microRNAs, compared to a healthy control, comprises a signature of a fibrotic lung disease; and
(c) medically managing the diagnosed fibrotic lung disease as early as possible in the course of progression of the disease to reduce or slow its progression by
   (1) treating the diagnosed fibrotic lung disease; and
   (2) monitoring over time the population of exosomes derived from the biological sample of the subject compared to a population of exosomes derived from a biological sample derived from a healthy control.

According to some embodiments, the lung function of the subject is quantified by determining forced expiratory volume (FEV1); forced vital capacity (FVC) and FEV/FVC %.

According to some embodiments, the biological sample is a body fluid. According to some embodiments, the body fluid is blood, breast milk, saliva, urine, bile, pancreatic juice, cerebrospinal fluid, amniotic fluid or a peritoneal fluid. According to some embodiments, the body fluid is serum or urine. According to some embodiments, the body fluid is serum. According to some embodiments, the body fluid is urine. According to some embodiments, the number of exosomes in the population of exosomes comprises at least 10E10 particles. According to some embodiments, the biological sample is obtained from a mammal. According to some embodiments, the biological sample is obtained from a non-human mammal or a human subject. According to some embodiments, the human subject is over 50 years of age. According to some embodiments, the healthy control is age and sex matched to the subject.

According to some embodiments, the population of exosomes comprises a total protein content of at least about 100-200 μg, inclusive, i.e., at least about 100 μg, at least about 105 μg, at least about 110 μg, at least about 115 μg, at least about 120 μg, at least about 125 μg, at least about 130 μg, at least about 135 μg, at least about 140 μg, at least about 145 μg, at least about 150 μg, at least about 155 μg, at least about 160 μg, at least about 165 μg, at least about 170 μg, at least about 175 μg, at least about 180 μg, at least about 185 μg, about 190 μg, at least about 195 μg, or at least about 200 μg.

According to some embodiments, the population of exosomes comprises a total RNA content of from at least about 100 ng to at least about 200 ng, inclusive, i.e., at least about 100 ng, at least about 105 ng, at least about 110 ng, at least about 115 ng, at least about 120 ng, at least about 125 ng, at least about 130 ng, at least about 135 ng, at least about 140 ng, at least about 145 ng, at least about 150 ng, at least about 155 ng, at least about 160 ng, at least about 165 ng, at least about 170 ng, at least about 175 ng, at least about 180 ng, at least about 185 ng, at least about 190 ng, at least about 195 ng, or at least about 200 ng.

According to some embodiments, selected miRNAs are expressed in the population of exosomes derived from subjects diagnosed with IPF when compared to healthy controls. According to some embodiments this selective miRNA expression can identify IPF at a stage before standard procedures (e.g., Ashcroft scoring and histology) demonstrate changes consistent with lung fibrosis.

According to some embodiments, the miRNA expression is a signature of a fibrotic lung disease. According to some embodiments:dysregulated miRNA expression of miR-199 and miR-34a in the population of exosomes derived from the biological sample of the subject compared to a population of exosomes derived from a biological sample derived from a healthy control is fibrotic. According to some embodiments:dysregulated miRNA expression of miR-142 in the population of exosomes derived from the biological sample of the subject compared to a population of exosomes derived from a biological sample derived from a healthy control is antifibrotic. According to some embodiments, dysregulated expression of miR-199 in the population of exosomes derived from the biological sample of a subject diagnosed with IPF compared to a population of exosomes derived from a biological sample derived from a healthy control is increased compared to a population of exosomes derived from a healthy control. According to some embodiments, the dysregulated expression of one or more of let-7D, miR-29, or miR-181 in the population of exosomes derived from the biological sample of the subject diagnosed with IPF compared to a population of exosomes derived from a biological sample derived from a healthy control is decreased, compared to the population of exosomes derived from the healthy control. According to some embodiments, dysregulated expression of miR-199 in the population of exosomes derived from the biological sample of the subject diagnosed with IPF compared to a population of exosomes derived from a biological sample derived from a healthy control and the dysregulated expression of one or more of let-7D, miR-29, miR-142, miR-181 derived from the population of exosomes derived from a biological sample from the subject diagnosed with IPF is decreased, compared to the population of exosomes derived from the healthy control. According to some embodiments, reduced or absent microRNA Let-7 expression in the population of exosomes derived from the biological sample of the subject compared to a population of exosomes derived from a biological sample derived from a healthy control is associated with early stage fibrotic lung disease. According to some embodiments, microRNA Let-7 expression in the population of exosomes derived from the biological sample of the subject compared to a population of exosomes derived from a biological sample derived from a healthy control is associated with end-stage fibrotic lung disease.

According to some embodiments, the purified enriched population of exosomes provides enhanced therapeutic targeting/precision; improved cell selectivity/tropism, and directed pharmacology at desired cellular or tissue sites. According to some embodiments, the purified enriched population of exosomes provides enhanced therapeutic targeting/precision. According to some embodiments, the purified enriched population of exosomes provides improved cell selectivity/tropism. According to some embodiments, the purified enriched population of exosomes provides directed pharmacology at desired cellular or tissue sites.

According to some embodiments, the progressive chronic lung disease if left untreated comprises one or more of a progressive injury, progressive inflammation, progressive fibrosis or a combination thereof.

According to some embodiments, the fibrotic lung disease is pulmonary fibrosis. According to some embodiments, the fibrotic lung disease is idiopathic pulmonary fibrosis. According to some embodiments, the chronic lung disease is due to chronic smoking or a severe viral infection. According to some embodiments the severe lung infection is due to a severe coronavirus infection. According to some embodiments, the dysregulated miRNAs expressed in the population of exosomes derived from subjects when compared to healthy controls are diagnostic of IPF. According to some embodiments the selective dysregulated miRNA expression can identify interstitial pulmonary fibrosis (IPF) at a stage before standard procedures (e.g., Ashcroft scoring and histology) demonstrate changes consistent with lung fibrosis.

According to some embodiments, the treating may modulate one or more of an injury, inflammation, an excess accumulation of extracellular matrix, cell senescence; or a pathway comprising fibrogenic signaling. According to some embodiments the treating of the progressive chronic lung disease may stabilize a parameter of lung function in the subject compared to an untreated control. According to some embodiments, the parameter of lung function is one or more of FEV1, FVC, or FEV1/FVC %. According to some embodiments the treating of the progressive chronic lung disease may improve a parameter of lung function in the subject compared to an untreated control.

According to some embodiments, the pathway comprises transforming growth factor (TGFβ) signaling. According to some embodiments, the pathway comprising fibrogenic signaling is one or more of a Smad pathway, a mitogen-activated protein kinase pathway, a phosphoinositide 3-kinase pathway; a canonical Wnt-β catenin pathway, a Notch signaling pathway.

### Formulations of Active Agents for Treating the Staged Fibrotic Lung Disease

According to some embodiments, a composition for treating the staged fibrotic lung disease is a pharmaceutical composition comprising a therapeutic amount of an active agent and a pharmaceutically acceptable carrier. The phrase “pharmaceutically acceptable carrier” is art recognized. It is used to mean any substantially non-toxic carrier conventionally useable for administration of pharmaceuticals in which the isolated EVs of the present invention will remain stable and bioavailable. The pharmaceutically acceptable carrier must be of sufficiently high purity and of sufficiently low toxicity to render it suitable for administration to the mammal being treated. It further should maintain the stability and bioavailability of an active agent. The pharmaceutically acceptable carrier can be liquid or solid and is selected, with the planned manner of administration in mind, to provide for the desired bulk, consistency, etc., when combined with an active agent and other components of a given composition. Exemplary carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Suitable pharmaceutical carriers are described in “Remington's Pharmaceutical Sciences” by E. W. Martin, which is incorporated herein by reference in its entirety. According to some embodiments, the pharmaceutically acceptable carrier is sterile and pyrogen-free water. According to some embodiments, the pharmaceutically acceptable carrier is Ringer's Lactate, sometimes known as lactated Ringer's solution.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, .alpha.-tocopherol, and the like; and metal chelating agents, such as citric acid, ethylenediamine tetraacetic acid (EDTA), sorbitol, tartaric acid, phosphoric acid, and the like.

Some examples of suitable carriers, excipients, and diluents include lactose, dextrose, sucrose, sorbitol, mannitol, starches, gum acacia, calcium phosphate alginates, calcium salicate, microcrystalline cellulose, polyvinylpyrrolidone, cellulose, tragacanth, gelatin, syrup, methyl cellulose, methyl- and propylhydroxybenzoates, tale, magnesium stearate, water, and mineral oil. According to some embodiments, the pharmaceutically acceptable carrier comprises a pulmonary surfactant. The formulations can additionally include lubricating agents, wetting agents, emulsifying and suspending agents, preserving agents, sweetening agents or flavoring agents. The compositions may be formulated so as to provide quick, sustained, or delayed release of the active ingredient after administration to the patient by employing procedures well known in the art.

Specific modes of administration will depend on the indication. The selection of the specific route of administration and the dose regimen is to be adjusted or titrated by the clinician according to methods known to the clinician in order to obtain the optimal clinical response. The amount of active agent to be administered is that amount sufficient to provide the intended benefit of treatment. The dosage to be administered will depend on the characteristics of the subject being treated, e.g., the particular mammal or human treated, age, weight, health, types of concurrent treatment, if any, and frequency of treatments, and can be easily determined by one of skill in the art (e.g., by the clinician).

The local delivery of therapeutic amounts of a composition for the treatment of a lung injury or fibrotic lung disease can be by a variety of techniques that administer the compound at or near the targeted site. Examples of local delivery techniques are not intended to be limiting but to be illustrative of the techniques available. Examples include local delivery catheters, site specific carriers, implants, direct injection, or direct applications, such as topical application and, for the lungs, administration by inhalation. Local delivery by an implant describes the surgical placement of a matrix that contains the pharmaceutical agent into the affected site. The implanted matrix releases the pharmaceutical agent by diffusion, chemical reaction, or solvent activators.

Pharmaceutical formulations containing the active agents of the described invention and a suitable carrier can be solid dosage forms which include, but are not limited to, tablets, capsules, cachets, pellets, pills, powders and granules; topical dosage forms which include, but are not limited to, solutions, powders, fluid emulsions, fluid suspensions, semi-solids, ointments, pastes, creams, gels, jellies, and foams; and parenteral dosage forms which include, but are not limited to, solutions, suspensions, emulsions, and dry powder; comprising an effective amount of a polymer or copolymer of the described invention. It is also known in the art that the active ingredients can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The means and methods for administration are known in the art and an artisan can refer to various pharmacologic references for guidance. For example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980) can be consulted.

The compositions for use according to the described invention relates to all routes of administration including intramuscular, subcutaneous, sublingual, intravenous, intraperitoneal, intranasal, intratracheal, topical, intradermal, intramucosal, intracavernous, intrarectal, into a sinus, gastrointestinal, intraductal, intrathecal, intraventricular, intrapulmonary, into an abscess, intraarticular, subpericardial, into an axilla, into the pleural space, intradermal, intrabuccal, transmucosal, transdermal, via inhalation, via nebulizer, and via subcutaneous injection. Alternatively, the pharmaceutical composition may be introduced by various means into cells that are removed from the individual. Such means include, for example, microprojectile bombardment, via liposomes or via other nanoparticle device.

The pharmaceutical compositions can be formulated for parenteral administration, for example, by injection, such as by bolus injection or continuous infusion. The pharmaceutical compositions can be administered by continuous infusion subcutaneously over a predetermined period of time. Formulations for injection can be presented in unit dosage form, e.g., in ampoules or in multi-dose containers, with an added preservative. The pharmaceutical compositions can take such forms as suspensions, solutions or emulsions in oily or aqueous vehicles, and can contain formulatory agents such as suspending, stabilizing and/or dispersing agents.

For oral administration, the pharmaceutical compositions can be formulated readily by combining the active agent(s) with pharmaceutically acceptable carriers well known in the art. Such carriers enable the actives of the disclosure to be formulated as tablets, pills, dragees, capsules, liquids, gels, syrups, slurries, suspensions and the like, for oral ingestion by a patient to be treated. Pharmaceutical preparations for oral use can be obtained by adding a solid excipient, optionally grinding the resulting mixture, and processing the mixture of granules, alter adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragecanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations that can be used orally include, but are not limited to, push-fit capsules made of gelatin, as well as soft, scaled capsules made of gelatin and a plasticizer, such as glycerol or sorbitol. The push-fit capsules can contain the active ingredients in admixture with filler such as, e.g., lactose, binders such as, e.g., starches, and/or lubricants such as, e.g., talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

For buccal administration, the compositions can take the form of, e.g., tablets or lozenges formulated in a conventional manner.

For administration by inhalation, the compositions for use according to the described invention can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

In addition to the formulations described previously, the compositions for use according to the described invention can also be formulated as a depot preparation. Such long acting formulations can be administered by implantation (for example subcutaneously or intramuscularly) or by intramuscular injection.

Depot injections can be administered at about 1 to about 6 months or longer intervals. Thus, for example, the compositions can be formulated with suitable polymeric or hydrophobic materials (for example as an emulsion in an acceptable oil) or ion exchange resins, or as sparingly soluble derivatives, for example, as a sparingly soluble salt.

Pharmaceutical compositions also can comprise suitable solid or gel phase carriers or excipients. Examples of such carriers or excipients include but are not limited to calcium carbonate, calcium phosphate, various sugars, starches, cellulose derivatives, gelatin, and polymers such as, e.g., polyethylene glycols.

For parenteral administration, a pharmaceutical composition can be, for example, formulated as a solution, suspension, emulsion or lyophilized powder in association with a pharmaceutically acceptable parenteral vehicle. Examples of such vehicles are water, saline, Ringer's solution, dextrose solution, and 5% human serum albumin. Liposomes and nonaqueous vehicles such as fixed oils may also be used. The vehicle or lyophilized powder may contain additives that maintain isotonicity (e.g., sodium chloride, mannitol) and chemical stability (e.g., buffers and preservatives). The formulation is sterilized by commonly used techniques.

Examples of such active therapeutic agents include one or more immunomodulators, analgesics, anti-inflammatory agents, anti-fibrotic agents, anti-viral agents, proton pump inhibitors, or oxygen therapy.

Examples of immunomodulators include corticosteroids, for example, prednisone, azathioprine, mycophenolate, mycophenolate mofetil, colchicine, and interferon-gamma 1b.

Examples of analgesics include capsaisin, codeine, hydrocodone, lidocaine, oxycodone, methadone, resiniferatoxin, hydromorphone, morphine, and fentanyl.

Examples of anti-inflammatory agents include aspirin, celecoxib, diclofenac, diflunisal, etodolac, ibuprofen, indomethacin, ketoprofen, ketorolac nabumetone, naproxen, nintedanib, oxaprozin, pirfenidone, piroxicam, salsalate, sulindac, and tolmetin.

Examples of anti-fibrotic agents are nintedanib and pirfenidone.

Examples of proton pump inhibitors are omeprazole, lansoprazole, dexlansoprazole, esomeprazole, pantoprazole, rabeprazole, and ilaprazole.

Examples of anti-viral agents include, for example, acyclovir, gancidovir, foscarnet; ribavirin; amantadine, azidodeoxythymidine/zidovudine), nevirapine, a tetrahydroimidazobenzodiazepinone (TIBO) compound; efavirenz; remdecivir, and delavirdine.

According to the foregoing embodiments, the pharmaceutical composition may be administered once, for a limited period of time or as a maintenance therapy over an extended period of time, for example until the condition is ameliorated, cured or for the life of the subject. A limited period of time may be for 1 week, 2 weeks, 3 weeks, 4 weeks and up to one year, including any period of time between such values, including endpoints. According to some embodiments, the pharmaceutical composition may be administered for about 1 day, for about 3 days, for about 1 week, for about 10 days, for about 2 weeks, for about 18 days, for about 3 weeks, or for any range between any of these values, including endpoints. According to some embodiments, the pharmaceutical composition may be administered for more than one year, for about 2 years, for about 3 years, for about 4 years, or longer.

According to the foregoing embodiments, the composition or pharmaceutical composition may be administered once daily, twice daily, three times daily, four times daily or more.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials have been described. All publications mentioned herein are incorporated herein by reference to disclose and described the methods and/or materials in connection with which the publications are cited.

It must be noted that as used herein and in the appended claims, the singular forms “a”, “and”, and “the” include plural references unless the context clearly dictates otherwise.

The publications discussed herein are provided solely for their disclosure prior to the filing date of the present application and each is incorporated by reference in its entirety. Nothing herein is to be construed as an admission that the present invention is not entitled to antedate such publication by virtue of prior invention. Further, the dates of publication provided may be different from the actual publication dates which may need to be independently confirmed.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

Abstract. Extracellular vesicles incorporate microRNAs (miRNA) and other cell-specific components that may convey disease to target cells. Alterations of miRNA expression have been shown in lung tissue, sputum and sera of individuals with idiopathic pulmonary fibrosis (IPF). We designed this study to test whether transfer of urine and/or tissue derived exosomal miRNAs from individuals with IPF carry cargo that can promote fibrosis. Exosomes were isolated from urine (U-IPFexo, n=16), lung tissue myofibroblasts (MF-IPFexo), and serum from individuals with IPF (n=8) and age/sex-matched controls without lung disease (n=10). We analyzed micro-RNA expression of isolated exosomes and their in vivo bio-distribution. We investigated the effect on ex vivo skin wound healing and in in vivo mouse lung models. U-IPFexo or MF-IPFexo expressed miR let-7d, miR-29a-5p, miR 181b-3p and miR-199a-3p consistent with previous reports of miRNA expression obtained from lung tissue/serum from patients with IPF. In vivo bio-distribution experiments detected bioluminescent exosomes in the lung of normal C57B16 mice within 5 minutes after intravenous infusion, followed by distribution to other organs irrespective of exosome source. When these exosomes were labeled with gold nanoparticles and imaged by transmission electron microscopy, they were visualized in alveolar epithelial type I and type II cells in lung tissue sections. Treatment of human and mouse lung punches obtained from control, non-fibrotic lungs, with either U-IPFexo or MF-IPFexo produced a fibrotic phenotype. A fibrotic phenotype was also induced in a human e vivo skin model and in in vivo lung models. Our results provide evidence that exosomes isolated from individuals with IPF contain pro-fibrotic miRNA and have the ability to promote a pro-fibrotic pathogenic response in normal and injured tissues.

### Materials and Methods

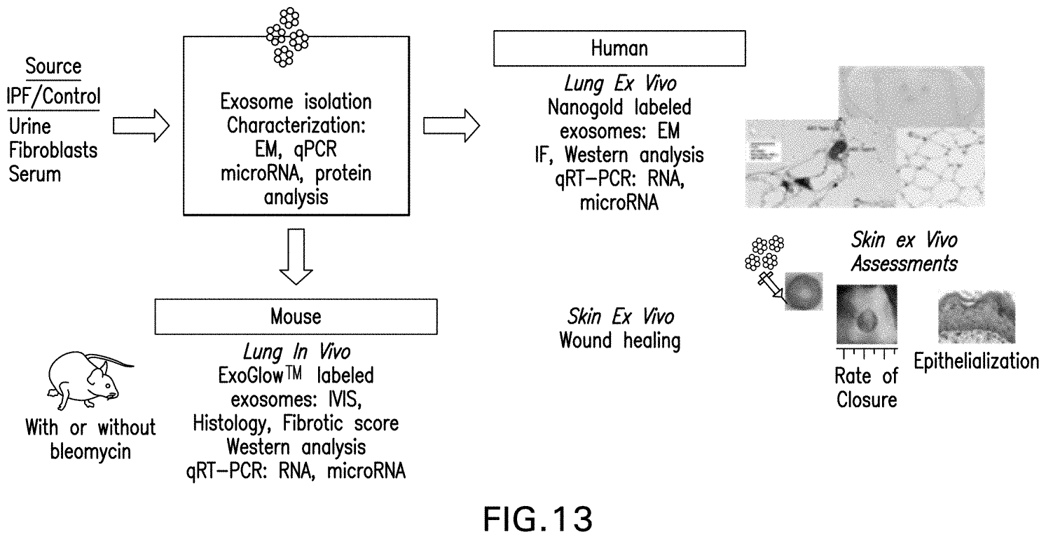

Study Design: This study aimed to determine whether urine-derived exosomes from individuals with IPF (U-IPFexo) carry disease provoking cargo. An overview of the experimental details and design is shown in FIG. 13.

Urine and blood collection: Random urine samples were collected (between 10 am-2 pm) from either control individuals or individuals seen in the Interstitial Lung Disease (ILD, subjects with IPF ages 55-79; subjects with non-CF bronchiectasis ages 73-86; and subjects with asthma ages 36-61) clinic at the University of Miami Hospital. Written informed consent was obtained from the participants with an approved Institutional Review Board protocol (IRB #20060249) at the University of Miami Miller School of Medicine. Control urines were obtained from subjects that did not have underlying kidney, heart or lung comorbidities and had normal albumin creatinine ratios sampled in an aliquot of each urine. Urine was processed within 3 hours of collection and spun at 3000×g for 15 mins to remove sediment; supernatant was aliquoted at 10 mls/tube. Patients with known kidney disease or diabetes were excluded from the study and a small aliquot of urine was used to measure urine albumin and creatinine to exclude samples with albuminuria (Creatinine LiquiColor Test, Stanbio Laboratory Boerne, Tex. and albumin Elisa, Bethyl laboratories, Montgomery, Tex.). Tubes were frozen at −80° C. until exosome isolation. Blood was collected during the same clinic visit as urine collection. Blood was left to clot for up to an hour and serum separated after centrifugation at ˜2500 RPM for 15 mins. Serum was aliquoted and frozen at −80° C. until exosome isolation. Samples were selected for inclusion in the present study after review of imaging confirming a definitive UIP pattern. Lung function (forced vital capacity (FVC), VEV1, FEV1:FVC %) and diffusion capacity (DLCO) performed at several facilities were obtained from subjects' medical records obtained closest to the timing of the sample was also reviewed as shown in Table 1.

Cell culture: Myofibroblasts isolated from individuals with IPF and fibroblasts isolated from control lungs were propagated and characterized as previously described. Two IPF and one control cell isolate was purchased from Lonza (catalog number CC7231 and CC2512, MSCBM, Lonza Inc., Walkersville, Md.) Cells (passages 2 or 3) were grown until 80% confluence in 30 T175 flasks in Lonza media (MSCBM, Lonza Inc., Walkersville, Md.). At the time of collection, media was aspirated and each flask washed 3 times with PBS to remove serum and serum proteins. After serum free medium was added back to each flask for an additional 48 hours, the supernatant was collected and exosomes isolated and characterized (See Zen Bio, NC method below).

Exosome Isolation (conditioned tissue culture medium, urine), characterization and RNA isolation: A 4° C. sample was centrifuged at 3,000×g for 20 min at room temperature in a swinging bucket rotor to remove large cells and debris. The clarified supernatant was collected and ultracentrifuged at 100,000×g for 2 hours, in a fixed angle rotor at 4° C., to pellet exosomes. The exosome pellet was resuspended in a minimum volume of DPBS (approximately 120 uL/ultracentrifugation tube). Exosomes were characterized using a Thermo NanoDrop spectrophotometer for protein determination and approximate RNA concentration by direct absorbance; exosomes were not lysed, stained, or RNA extracted prior to taking these measurements. Particle diameter and concentration was assessed by tunable resistive pulse sensing (TRPS; (qNano, Izon Science Ltd) using a NP150 nanopore membrane at a 47 mm stretch. The concentration of particles was standardized using multi-pressure calibration with carboxylated polystyrene beads of a defined size (nm diameter) and at a defined concentration (particles/mL).

For RNA isolation the Norgen Preserved Blood RNA Purification Kit I was used according to manufacturer's directions. Briefly, for every 100 μl of exosome preparation, 300 μL of lysis solution was added. After a short vortex, 400 μL of 95-100% ethanol was added. 600 μl of the lysate containing ethanol was loaded onto the column and centrifuged for 1 minute at >3,500×g (˜6,000 RPM). The column was washed three times with wash solution and centrifuged each time for 1 minute (14,000×g). The column was spun for an additional two minutes in order to thoroughly dry the resin and the column contents eluded with 50 μl of Elution Solution by centrifuging for 2 minutes at 200×g (˜2,000 RPM), followed by 1 minute at 14,000×g.

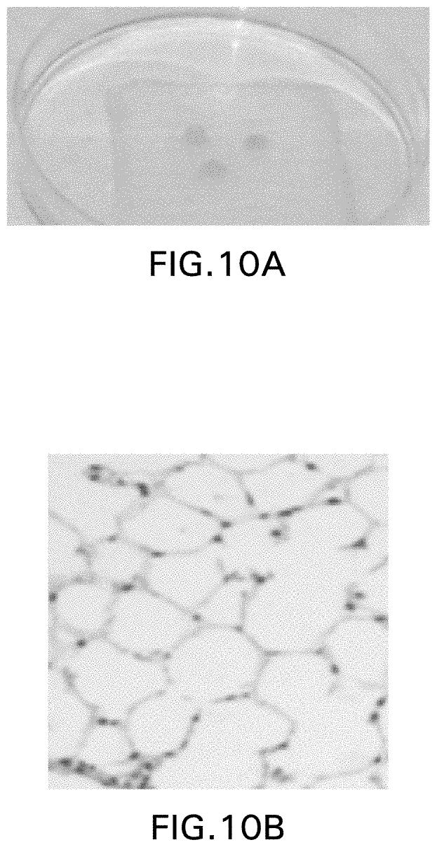

Lung Punch: To test the effect of exosomes on fibrotic pathways, we used ex vivo lung punches obtained from the lungs of 17-18-month-old male C57BL/6 mice (FIG. 10A). Naïve exosomes (40 ug) and exosomes labeled with nanoparticles were injected around the punch followed by collection four days later. Punches were embedded, cut and stained with trichrome. The normal histology of the punch was preserved as shown by light and electron microscopic examination after PBS injection (FIG. 10B and FIG. 10C).

Transmission Electron Microscopy: For conventional transmission electron microscopy (TEM), exosome pellets were placed in a droplet of 2.5% glutaraldehyde in PBS buffer at pH 7.2 and fixed overnight at 4° C. The samples were rinsed in PBS (3 times, 10 minutes each) and post-fixed in 1% osmium tetroxide for 60 minutes at room temperature. The samples were then embedded in 10% gelatin, fixed in glutaraldehyde at 4° C. and cut into several blocks (smaller than 1 mm³). The samples were dehydrated for 10 minutes per step in increasing concentrations of alcohol (30%, 50%, 70%, 90%, 95% and 100%×3). Next, pure alcohol was replaced with propylene oxide, and the specimens were infiltrated with increasing concentrations (25%, 50%, 75% and 100%) of Quetol-812 epoxy resin mixed with propylene oxide for a minimum of 3 hours per step. The samples were embedded in pure, fresh Quetol-812 epoxy resin and polymerized at 35° C. for 12 hours, 45° C. for 12 hours, and 60° C. for 24 hours. Ultrathin sections (100 nm) were cut using a Leica UC6 ultramicrotome and post-stained, first with uranyl acetate for 10 minutes and then with lead citrate for 5 minutes at room temperature, prior to observation using the FEI Tecnai T20 transmission electron microscope (Hillsboro, Tex., USA) operated at 120 kV.

Punch TEM: Individual punches were fixed in 2% glutaraldehyde in 0.05M phosphate buffer and 100 mM sucrose, post-fixed overnight in 1% osmium tetroxide in 0.1M phosphate buffer, dehydrated through a series of cold graded ethanol, and embedded in a mixture of EM-bed/Araldite (Electron Microscopy Sciences). 1 μm thick sections were stained with Richardson's stain for observation under a light microscope. 100 nM sections were cut on a Leica UC7 ultramicrotome and stained with uranyl acetate and lead citrate. The grids were viewed t 80 kV in a JEOL JEM-1400 transmission electron microscope and images captured by an AMT BioSprint 12 digital camera.

Biodistribution: Mice were fed an alfalfa-free diet to prevent auto fluorescence of tissues (Perkin-Elmer personal communication). Exosomes were labeled with ExoGlow™-Vivo EV Labeling Kit (System Biosciences, Palo Alto, Calif.). Labeled exosomes or PBS containing exosomes were injected via tail vein into 17-18 month-old C57BL/6 mice. Mice were anesthetized with isoflurane and imaged using an IVIS Spectrum In Vivo Imaging System (PerkinElmer). The mice were imaged at 5, 10, 15, 30, 60, 90 mins, 2, 4, 6, and 24 hours as well as at 2, 3 and 19 days post injection. Imaging data was analyzed using Living Image Software (Perkin Elmer). Control experiments were conducted with dye alone.

Gold nanoparticle labelling of exosomes: Gold nanoparticles (nanospheres) modified with branched polyethylenimine (BPEI) of 10 nm size were used (nanoComposix, co). The original concentration of gold nanoparticles purchased was 1 mg/mL (5.7E+13 particles/mL). We performed experiments to optimize the ratio between the exosome particles and the modified gold nanoparticles with the final prep containing 10*8 exosomes: 0.001 mg of the modified gold nanoparticles. Briefly, gold nanosuspensions were sonicated prior to the experiment. The concentration of gold nanoparticles was adjusted based on the concentration of each exosome prep and the desired volume for injection. The mixture was vortexed and then placed in a thermomixer (Eppendorf ThermoMixer F1.5) at 37° C. and spun at 300 rpm. After 3 hours, the mixture was vortexed, allowed to sit at room temperature for 15 mins, and placed at 4° C. until the next day.

Transmission electron microscopy: For conventional transmission electron microscopy (TEM), exosome pellets were placed in a droplet of 2.5% glutaraldehyde in PBS buffer at pH 7.2 and fixed overnight at 4 C. The samples were rinsed in PBS (3 times, 10 minutes each) and post-fixed in 1% osmium tetroxide for 60 minutes at room temperature. The samples then were embedded in 10% gelatin, fixed in glutaraldehyde at 4 C and cut into several block (smaller than 1 mm³). The samples were dehydrated for 10 minutes per step in increasing concentrations of alcohol (30%, 50%, 70%, 90%, 95%, and 100%×3). Next, pure alcohol was replaced with propylene oxide, and the specimens were infiltrated with increasing concentrations (25%, 50%, 75% and 100%) of Quetol-812 epoxy resin mixed with propylene oxide for a minimum of 3 hours per step. The samples were embedded in pure, fresh Quetol-812 epoxy resin and polymerized at 35 C for 12 hours, 45 C for 12 hours, and 60 C for 24 hours. Ultrathin sections (100 nm) were cut using a Leica UC6 ultramicrotome and post-stained, first with uranyl acetate for 10 minutes and then with lead citrate for 5 minutes at room temperature, prior to observation using a JEOL JEM-1400 transmission electron microscope operated at 120 kV.

Ex Vivo lung punches: Cannulated lungs from 17-18 month-old male mice were filled with warm low melting point agarose (3%, Sigma-Aldrich, St. Louis, Mo.). Lung segments were cooled on ice for 30 min to allow solidification of the agarose, punched to a diameter of 4 mm using a biopsy punch (Acuderm) and transferred to an air-liquid interface with phenol red free minimal essential media (MEM) without serum. Human urine-derived or cell derived exosomes were injected into the lung punch with 29G needles in a volume of 100 μl. The 100 ul volume of injection was divided equally into four injection sites on the punch. Punches were incubated at 37° C. in a humidified atmosphere of 5% CO₂ for 4 days. Parallel punches (injected and naïve) from the same experiment were prepared for histology, immunofluorescence staining, RNA and protein purification. The normal histology of the punch was preserved after injection with PBS at four sites. For human lung, we injected a bronchial branch with 2% agarose and performed punch experiments as described above. Selected punches were injected with nanoparticle containing exosomes for EM experiments and collected within 24 hours of injection. A series of punches were collected that did not receive any injections or were injected with nanoparticles alone. Four days after injection punches were fixed in 10% formalin (Sigma-Aldrich), processed for paraffin embedding and stained with trichrome to assess collagen content.

Punch TEM: Individual punches were fixed in 2% glutaraldehyde in 0.05M phosphate buffer and 100 mM sucrose, post-fixed overnight in 1% osmium tetroxide in 0.1 M phosphate buffer, dehydrated through a series of cold graded ethanol, and embedded in a mixture of EM-bed/Araldite (Electron Microscopy Sciences). 1 μm thick sections were stained with Richardson's stain for observation under a light microscope. 100 nm sections were cut on a Leica UC7 ultramicrotome and stained with uranyl acetate and lead citrate. The grids were viewed at 80 kV in a JEOL JEM-1400 transmission electron microscope and images captured by an AMT BioSprint 12 digital camera.

Immunofluorescence staining of lung punches: Formalin fixed paraffin embedded punches were processed as previously described for tissue. Fluorescent staining was performed using α-SMA (Abcam, Cambridge, Mass.) or anti-prosurfactant protein C (SPC, Abcam) and DAPI (Vector, Burlingame Ca).

Real-Time PCR: Total RNA was extracted from lung tissue homogenates. Amplification and measurement of target RNA was performed on the Step 1 real time PCR system as previously described. αᵥ-integrin, collagen type I and TGF-β were measured using RNA extracted from lung punches or lung tissue. TGF-β and IGF-1 mRNA expression was measured using RNA extracted from exosomes. The TaqMan rRNA control reagents kit (Life Technologies) was used to detect 18S rRNA gene, an endogenous control, and samples were normalized to the 18S transcript content as previously described. For miRNA analyses, cDNA was generated using qScript™ microDNA cDNA Synthesis Kit (Quanta Biosciences, Beverly, Mass.) according to manufacturer's instructions. Amplification of all miRNAs was performed using specific primers, let-7d, miR-29a-5p, miR-34a-5p, miR-142-3p, miR-199a-3p, and miR-181b (IDT, Coralville, Iowa) using Real-Time SYBR Green qRT-PCR Amplification kit (Quanta Biosciences, Beverly, Mass.). U6 expression was used as a control for miRNA analyses, and relative expression was calculated using the comparative C(T) method.

Western Blot: Punches or lung pieces were homogenized and western analysis was performed as previously described using the Invitrogen mini-cell gel surelock cell module Xcell II vertical surelock box (Thermofisher, Waltham, Mass.). For CD63, Hsp70, pAKT, AKT, c-Jun, Caveolin-1, ERα and β-actin, 5 to 25 μg of protein lysate was fractionated on 10% polyacrylamide gels. Immunoreactive bands were determined by exposing nitrocellulose blots to a chemiluminescence solution (Denville Scientific Inc.; Metuchen, N.J.) followed by exposure to Amersham Hyperfilm ECL (GE Healthcare Limited; Buckinghamshire, UK). To determine the relative amounts of protein densitometry Image J version 1.48v (National Institutes of Health; Bethesda, Md.) was utilized. All values from western blots were standardized to the corresponding β-actin band prior to comparative analyses.

### MMP Activity:

MMP activity was assessed in lung punches and lung tissue using a previously described method. Briefly, Novex® 10% zymogram gels (Life Technologies) were incubated for 24 hours in a gelatinase solution, which allows the determination of total proteolytic MMP activities without interference from associated tissue inhibitors. Relative MMP activity was determined by densitometry using Image J (NIH).

Ex vivo human skin wound model to evaluate functional effects of urine-derived exosomes: Human skin samples were obtained from healthy subjects following panniculectomy (abdominal skin; median age 44 years old). Informed consent was obtained per the requirements of the Institutional Review Board at the University of Miami (IRB protocol #20070922). Under sterile conditions, subcutaneous fat was trimmed from skin prior to generating wounds. A 3 mm punch (Acuderm) was used to make wounds in the epidermis through the reticular dermis and 3 mm discs of eipidermis were excised. Skin discs (8 mm), with the 3 mm epidermal wound in the middle, were excised using a 6 mm biopsy punch (Acuderm). Wounded skin specimens were immediately transferred to anir-liquid interface with DMEM medium (BioWhittaker) supplemented with antibiotics-antimycotics and 10% fetal bovine serum (Gemini Bio—Products). The skin samples were incubated at 37 C in a humidified atmosphere of 5% CO2 for 4 days. Tissues were fixed in 10% formalin (Sigma-Aldrich), processed for paraffin embedding and stained with hematoxylin and eosin to follow the rate of healing.

### Animal Model:

17-18 month-old male C57BL/6 mice obtained from Jackson Laboratories were housed under specific pathogen-free conditions with food and water ad libitum. All experiments and procedures were approved by the Institutional Animal Care and Use Committee at the Leonard M. Miller School of Medicine at the University of Miami (Miami, Fla.), a facility accredited by the American Association for the Accreditation of Laboratory Animal Care. Following treatments all mice were housed one per cage until sacrifice. Sample size was based on our published data (72). Starting with 8-10 mice per group we have >90% statistical power to detect a Cohen's effect size d>1.75 standard deviations.

Bleomycin (“Bleo”) administration: After the administration of anesthesia, Bleomycin sulfate (Sigma-Aldrich Corp; St. Louis, Mo.) dissolved in 50 μl sterile saline at 2.5 U per kg of bodyweight was administered by direct intratracheal instillation via intubation. Control mice received 50 μl of sterile saline using the same method. Mice were weighed and sacrificed at 21 days post-Bleo administration.

### Exosome Injections and Time Course:

Urine-derived or cell-derived exosomes were thawed in a 37° C. water bath and washed in PBS to remove the cell freezing solution immediately prior to injection. Twenty-four hours following Bleo administration, each animal received 100 μl either PBS (control) or 40 ug of exosomes in 100 μl of PBS by tail vein injection over a one minute period. This amount was calculated based on the amount of exosomes derived from 10⁵ cells (number of cells utilized in whole cell experiments). Some mice received Bleo+vehicle injection. Treatments were assigned by simple randomization and the technician was blinded to the treatments. Dose response experiments were conducted with 20 and 40 ug of exosomes. A separate set of naïve mice received the same exosome injections and were sacrificed at 21 days post injection.

### Marine Lung Tissue Analysis/Immunohistochemistry:

Left lung lobes were harvested for protein, zymography, and mRNA analysis. For morphometry and histology studies, right lung lobes were inflated with 10% neutral buffered formalin (NBF) under 25 cm H₂O pressure. The lungs were postfixed by immersion in 10% NBF for 24 hours and then transferred to PBS at 4° C. Samples were paraffin-embedded and 4 m sections were obtained for hematoxylin-eosin and Masson's Trichrome staining, Ashcroft scoring:

Pulmonary fibrosis was assessed by a pulmonary pathologist blinded to the experimental groups using the semi-quantitative Ashcroft method on Masson's Trichrome-stained slides at 20× magnification. Individual fields were assessed by systematically moving over a 32-square grid; each field was assessed for fibrosis severity and assigned a score on a scale of 0 (normal lung) to 8 (total fibrosis of the field) and an average was obtained for each slide.

[Collagen content assessment by hydroxyproline content: Hydroxyproline content was determined according to the manufacturer's instructions (Hydroxyproline Assay Kit; Sigma-Aldrich, St. Louis, Mo.). Briefly, 2 mg lung fragments were weighed and homogenized in 100 μl of distilled water. An equal volume of 10 N HCl was added to the samples before drying at 49° C. for 3 hours. 50 μl of sample was loaded in the plate and incubated overnight at 37° C. A hydroxyproline standard curve was prepared according to a standard solution (0-1 ug/well). Hydroxyproline content was read at 557 nm, using the SoftMax Pro Software (Molecular Devices Corp; Sunnyvale, Calif.).

Statistical analysis: Mean and SEM were determined using GraphPad Prism 9.0 (GraphPad Software, San Diego, Calif.). All values are expressed as mean±SEM. Differences between experimental groups were assessed by using Kruskal-Wallis test and Mann-Whitney test for single comparisons. Given limited sample sizes in some experiments, data were determined to be normally distributed using Kolmogorov-Smirnov test and tested by one-way analysis of variance (ANOVA) and Tukey multiple comparison. Results were considered statistically significant at P<0.05. ANOVA was also used to analyse rate of epithelialization among treatment groups; p<0.05 was considered significant.

### Results

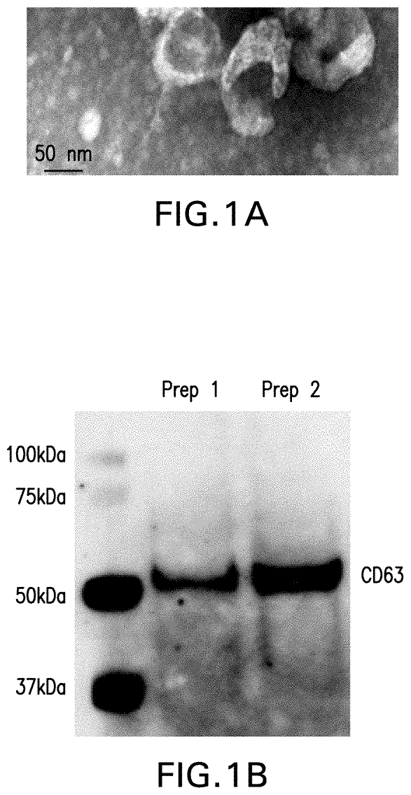

Exosome isolation and characterization: Urine samples were collected from individuals diagnosed with IPF (A group) or non-CF bronchiectasis (B group) or asthma (C group, Table 1) and normal age and sex-matched controls (Table 2, IRB #20060249). Control urines were obtained from subjects that did not have underlying kidney, heart, or lung comorbidities and had normal albumin creatinine ratios sampled in an aliquot of each urine. IPF and control exosomes were isolated from myofibroblasts and fibroblasts (Table 3). Human urine, blood and cell derived exosome isolation were performed by Zen-Bio Inc. (Durham, N.C.). Exosome size from urine, blood and cells was approximately 30-150 um. EM performed on isolated exosomes (FIG. 1A) confirmed a size and shape previously reported for exosomes (23). The presence of exosome markers CD1, CD9 and CD63 was determined by MACSPlex analysis and CD63 (FIG. 1B) was confirmed by Western analysis. Other exosome markers present were CD9 and CD81.

TABLE 1
Male IPF (A group), non-CF bronchiectasis (B group)
or asthma (C group) urine-derived exosomes.

| | Age of | | | | | | | |
|---|---|---|---|---|---|---|---|---|
| Subject | subject at | | FEV1 | FVC | FEV/FVC | FEV/FVC | DLCO (% | |
| Number | collection | Ethnicity | (liters) | (liters) | (%) | (predicted) | reference) | |
| A4 | 72 | Caucasian | 2.93 | 3.75 | 78 | 77 | 11.8 | (47) |
| A6 | 79 | Caucasian | 2.36 | 2.91 | 81 | 67 | 16.2 | (101) |
| A26 | 69 | Hispanic | 1.67 | 1.98 | 84 | 72 | NT | |
| A35 | 69 | Hispanic | 1.71 | 2.22 | 70 | 73 | NT | |
| A37 | 69 | Hispanic | 2.7 | 3.21 | 84 | 77 | 10.5 | (44) |
| A62 | 67 | Hispanic | 1.94 | 2.33 | 82 | 77 | 8.7 | (33) |
| A74 | 75 | Hispanic | 2.48 | 2.77 | 80 | 74 | 9.9 | (49) |
| A77 | 55 | Hispanic | 1.14 | 1.27 | 90 | 78 | 6.7 | (32) |
| A80 | 70 | Caucasian | 2.09 | 2.62 | 78.9 | 87.49 | 11.8 | (48) |
| A83 | 68 | Hispanic | 2.39 | 2.69 | 86 | 75 | 10.5 | (49) |
| A84 | 67 | Caucasian | 1.78 | 2.12 | 84 | 84 | 14.1 | (57) |
| A88 | 66 | Hispanic | 1.36 | 1.39 | 98 | 75 | 2.6 | (11) |
| A90 | 67 | Caucasian | 2.48 | 2.85 | 88.9 | 74 | 12.8 | (45) |
| A103 | 72 | Hispanic | 1.95 | 2.64 | 74 | 75 | 13.4 | (59) |
| A104 | 76 | Caucasian | 2.95 | 3.27 | 90 | 65 | 14.6 | (58) |
| A105 | 62 | Hispanic | 1.47 | 1.69 | 87 | 78 | 11.7 | (41) |
| B1 | 73 | Caucasian | 1.04 | 2.06 | 50 | 79.91 | 9.6 | (48) |
| B10 | 70 | Hispanic | 1.33 | 2.89 | 46 | 74 | 21.29 | (69.5) |
| B13 | 86 | Hispanic | 2.54 | 3.59 | 70.7 | 74 | 23.08 | (76.78) |
| C1 | 36 | Hispanic | 3.67 | 4.75 | 77.16 | 82.89 | 31.49 | (92) |
| C4 | 61 | Caucasian | 3.38 | 4.72 | 71.65 | 79.09 | 27.77 | (96) |
| C7 | 44 | Caucasian | 3.08 | 4.57 | 67.3 | 70 | 27.83 | (96) |

NT = not tested.

TABLE 2
Male control (D group) urine derived exosomes (Age of
subject at collection). No evidence of documented lung
disease or abnormal pulmonary function tests (PFTs).

| | Age of subject at | |
|---|---|---|
| Subject number | collection | Ethnicity |
| C8 | 66 | Caucasian |
| D9 | 70 | Caucasian |
| D12 | 77 | Hispanic |
| D28 | 77 | Caucasian |
| D31 | 55 | Hispanic |
| D32 | 73 | Caucasian |
| D38 | 72 | Hispanic |
| D41 | 65 | Caucasian |
| D50 | 75 | Hispanic |
| D101 | 67 | Caucasian |

TABLE 3
Myofibroblast and control fibroblast-derived
exosomes (age of subject at collection)

| Male myofibroblasts IPF | Male fibroblast Control |
|---|---|
| (Age of subject at collection) | (Age of subject at collection) |
| 1 (52) | 5 (70) |
| 2 (83) | 6 (69) |
| 3 973) | 7 967) |
| 4 974) | |

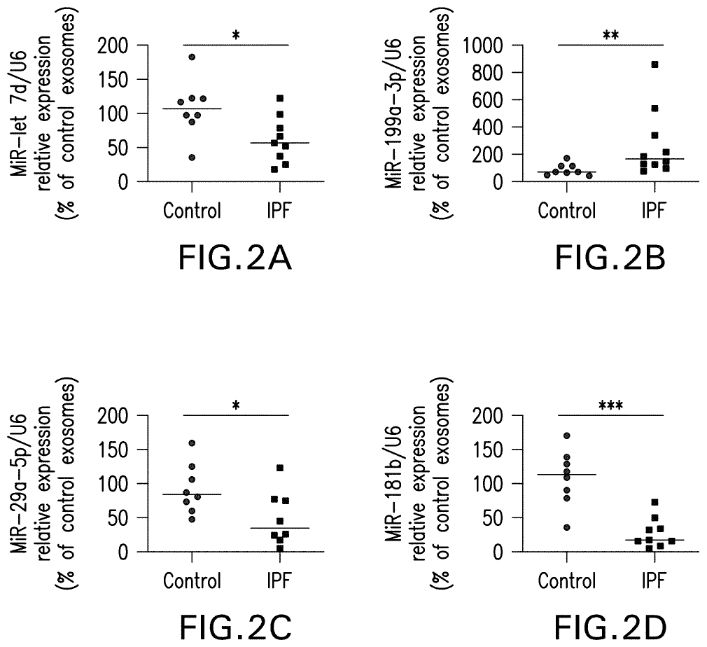

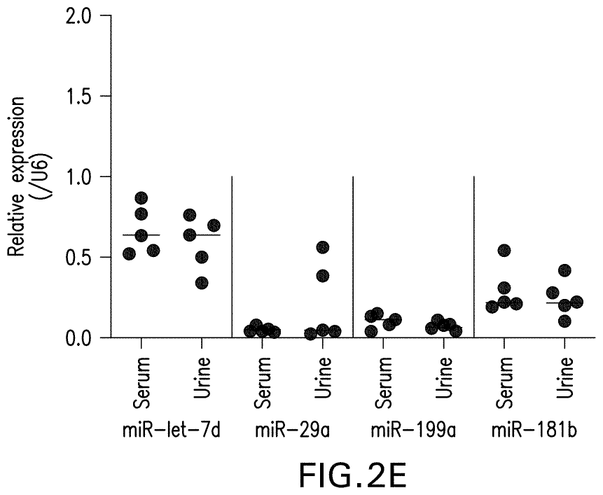

Determination of miRNAs expressed in urine- and serum-derived exosomes: to our knowledge, determination of the cargo of U-IPFexo has not previously been reported. U-IPFexo relative expression analysis revealed dysregulated expression of miR-let-7D, miR-199, miR-29 and miR-181 (FIG. 2A, FIG. 2B, FIG. 2C, FIG. 2D) compared to urine-derived control exosomes. We compared serum-derived exosome miRNA to urine-derived exosomal miRNA from the same patient and found no difference in relative expression of selected miRNAs (FIG. 2E). These selected miRNAs have also been shown to be altered in lung tissue and serum from individuals with IPF (Cassanova, N. et al. Translational Research (2021) 228: 1-12; Omote, N. and Sauler, M. Frontiers in Medicine (2020) 7 (908); Ortiz-Quinntero, B. et al. Cells (2020)). These data suggest that cargo carried in urine exosomes from individuals with IPF may parallel cargo of serum-derived exosomes in the same individual.

Detection of expression of mRNA from exosomes: Isolated exosomes were subject to PCR to detect potential mRNA expression that is reflective of fibrotic pathways. As expected in pure exosome preparations (Batagov, A O and Kurochkin, IV (20013) Biology Direct (2013) 8: 12), we did not detect mRNA expression of TGFβ, IGF or ER subtypes from normal or diseased exosomes.

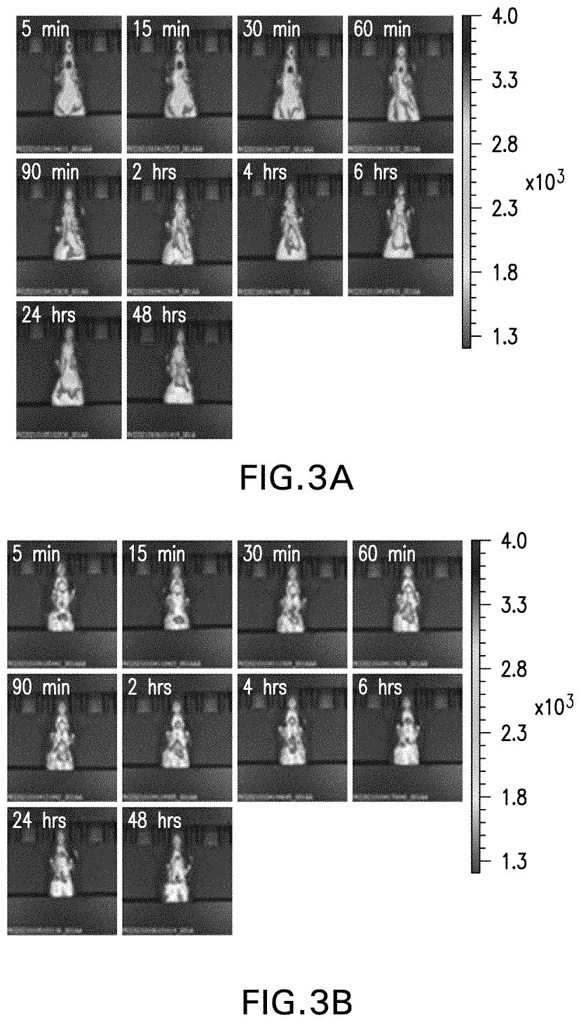

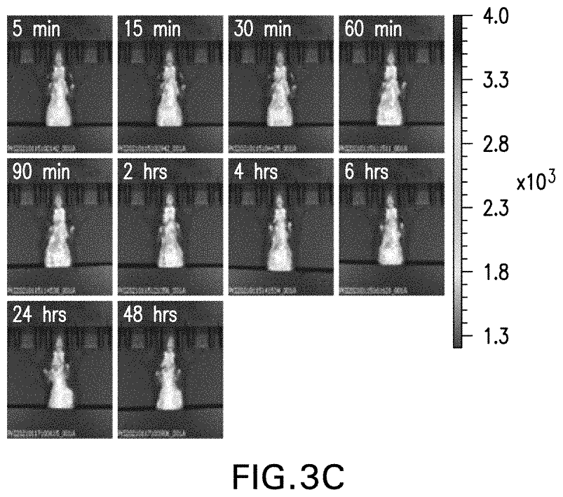

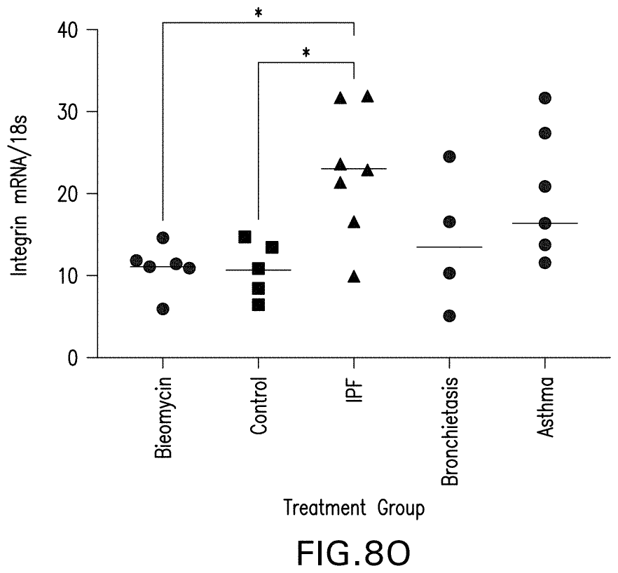

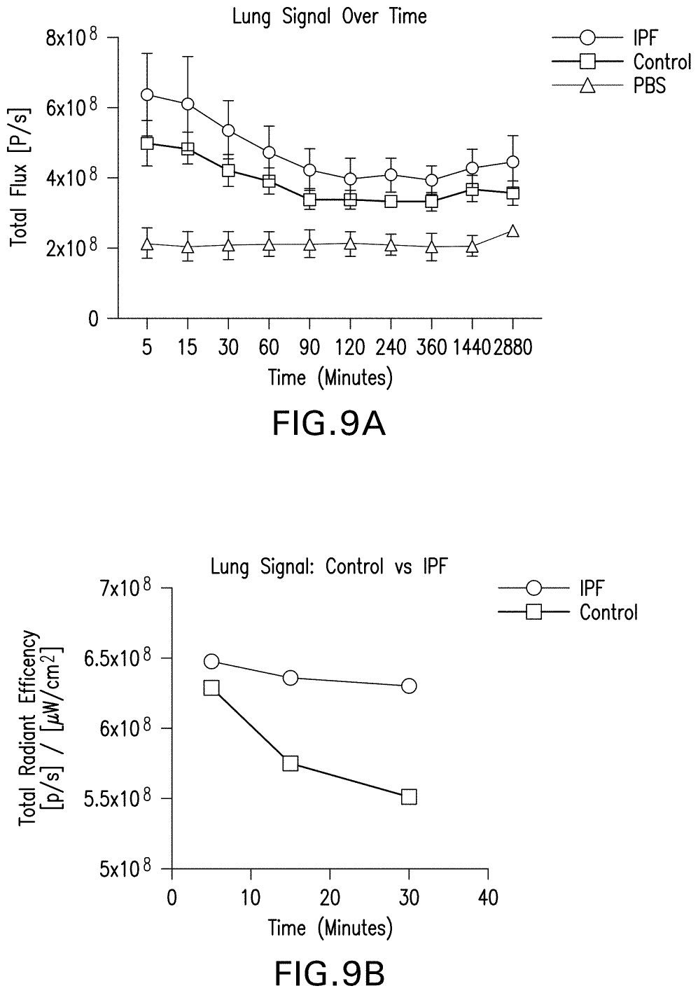

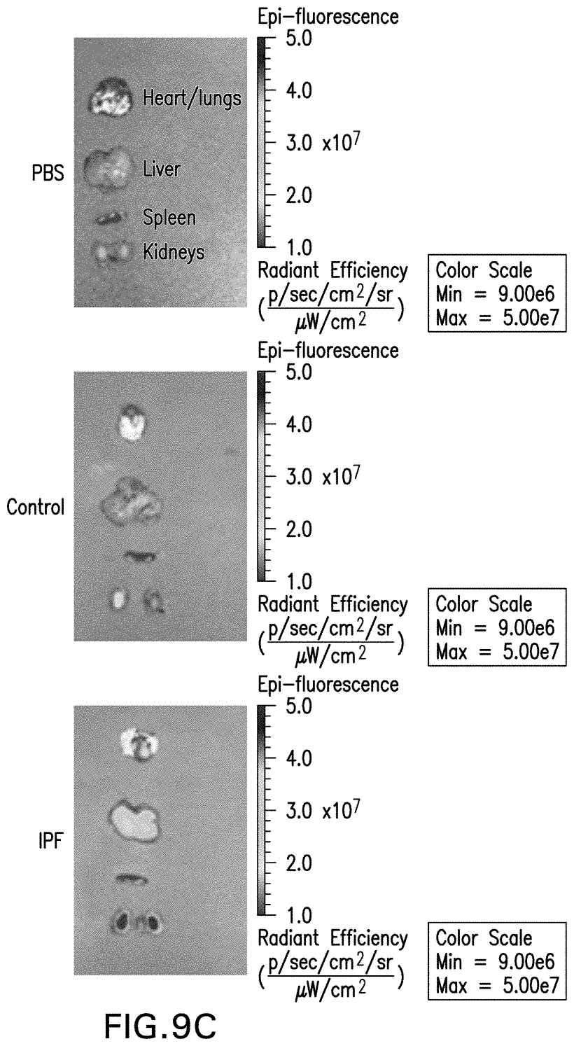

Tracking exosomes reveals a rapid systemic distribution: We assessed exosome signal and time course in 17-18 month-old male C57BL/6 mice using an in vivo bioluminescent imaging system. Regardless of source (U-IPFexo or control exosomes), exosomes were located the lung within 5 minutes post-tail vein injection. Images were taken at 5 min, 15 min 30 min, 60 min, 90 min and 2, 4, 6, 24 and 48 hours (FIG. 3) in all preparations. No adverse events were noted in mice followed for 20 days. All mice injected with U-IPFexo had a higher lung signal over time compared to mice injected with urine-derived age and sex-matched controls (FIG. 9A), suggesting that these exosomes may be retained longer in the lung than control exosomes. A set of mice sacrificed at 30 min or 48 hours to quantify signals in heart/lung, spleen, liver and kidney (FIG. 9B) showed that the lung signal evident at 30 min was not evident at 48 hours as seen by IVIS suggesting clearance of the exosomes. Mice injected with PBS had no signal (FIG. 9C).

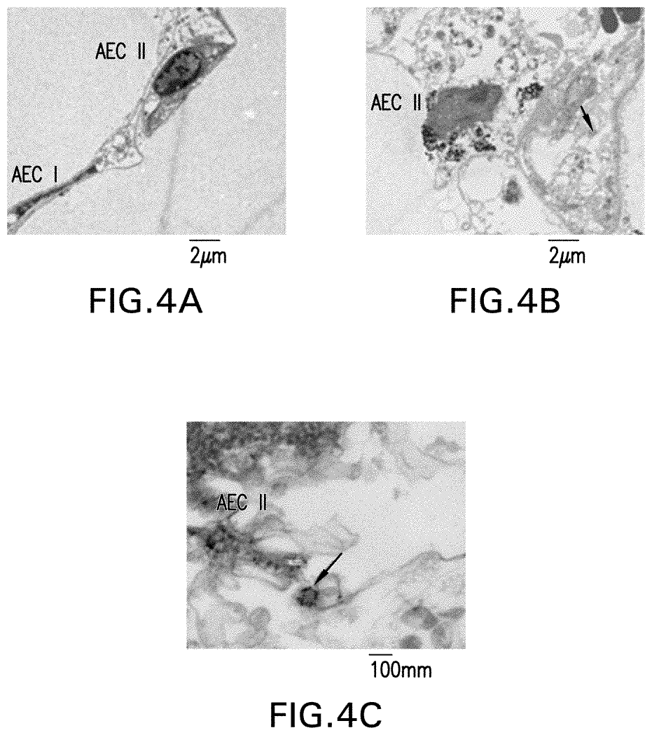

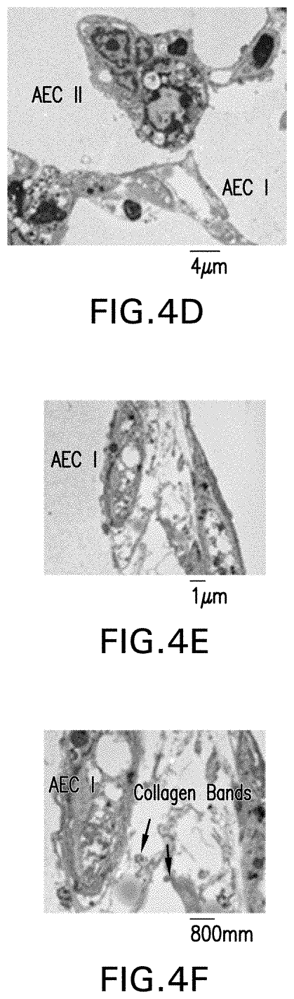

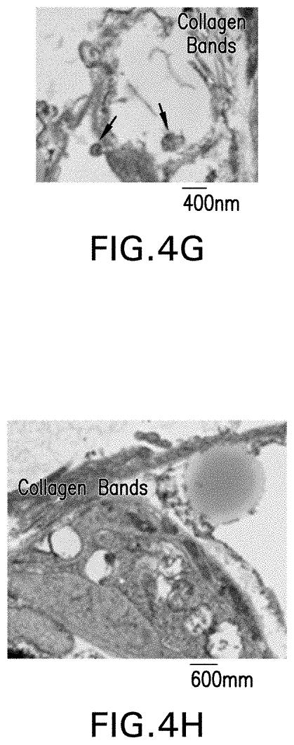

Transmission Electron Microscopy (TEM) reveals uptake of exosomes into alveolar epithelial cells (AEC): Exosomes were labeled with gold nanoparticles at varying ratios and processed for EM to determine the optimum ratio of nanoparticles to exosomes. Utilizing TEM, exosomes labeled with nanoparticles were visualized in mouse lung punches (FIG. 10) injected with urine-derived control exosomes (FIG. 4A, FIG. 4B, FIG. 4C) or U-IPFexo (FIG. 4D, FIG. 4E, FIG. 4F, FIG. 4G, FIG. 411). FIG. 10A, FIG. 10B, FIG. 10C and FIG. 10D show histology and trichrome staining of lung punches from C57BL6 mice. Lung punches from control lungs shown in tissue culture dish (FIG. 10A) have normal histology (FIG. 10B, Trichrome staining 10×mag) and structure by TEM (FIG. 10C, 500 mag). Histology of non-injected lung punch (FIG. 10D). TEM revealed increased collagen bands in those punches injected with U-IPFexo (FIG. 411), Collagen bands were not as evident in the control punches as compared with U-IPF samples due to sampling limitation, however we observed collagen bundles in both normal and U-IPF treated samples. Lung structure was preserved in both normal and U-IPF treated samples.

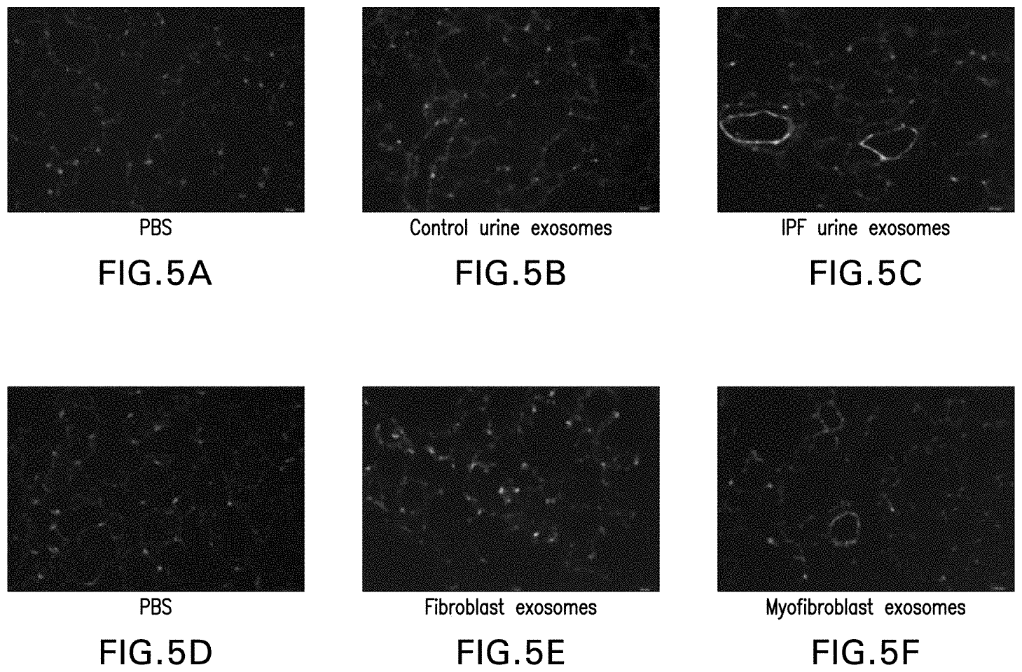

Immunofluorescent staining of punches revealed decreased surfactant protein C positive cells (AEC II) after treatment with IPF derived exosomes (FIG. 5C) compared to treatment with control exosomes (FIG. 5B). Taken together these data suggest that exosomes are taken up by the alveolar cells and that IPFexo may promote disease by inhibiting the reparative AEC II cells.

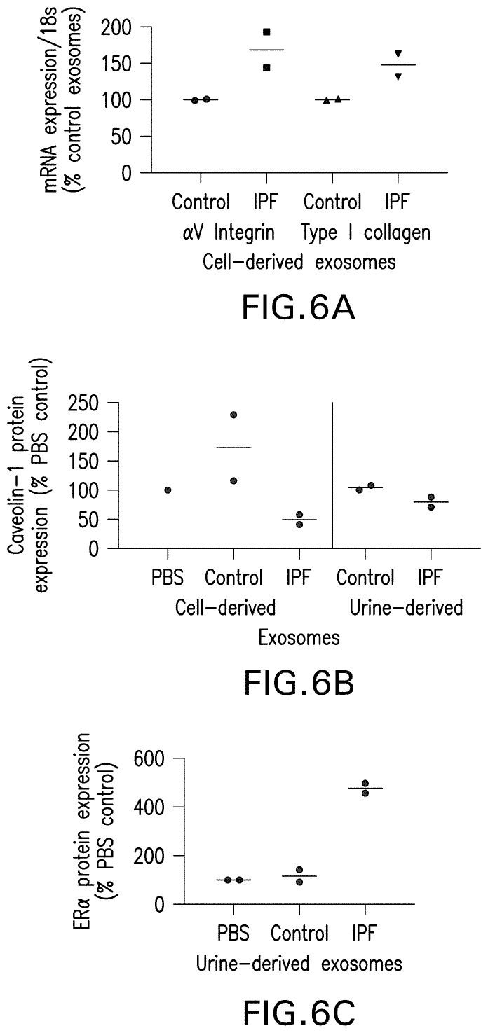

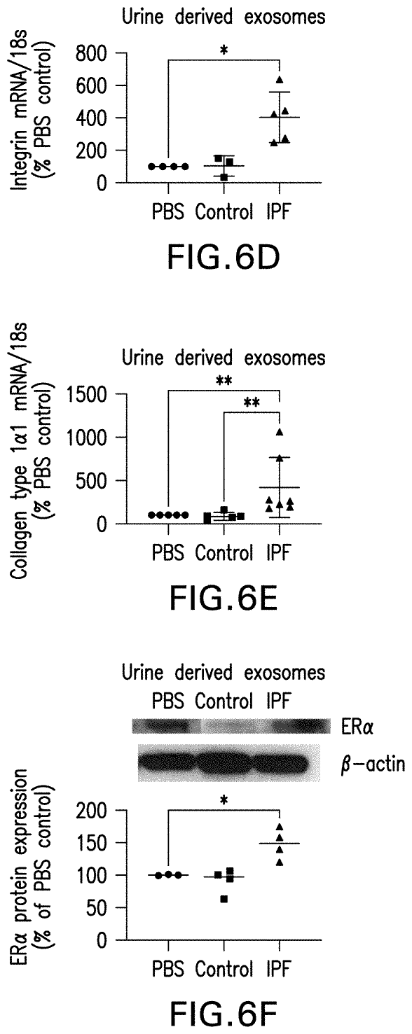

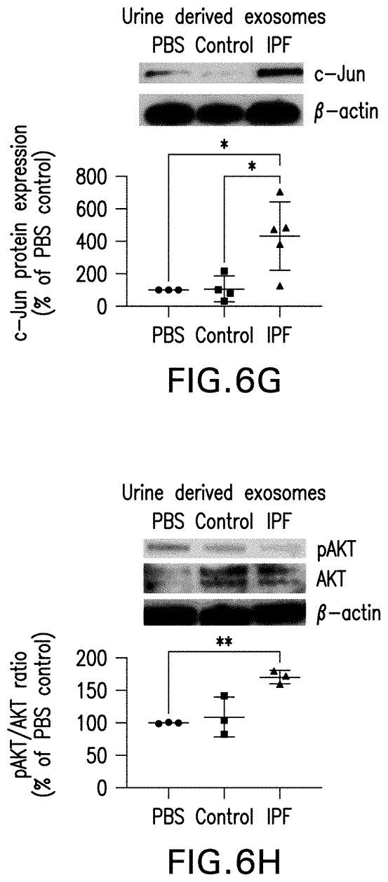

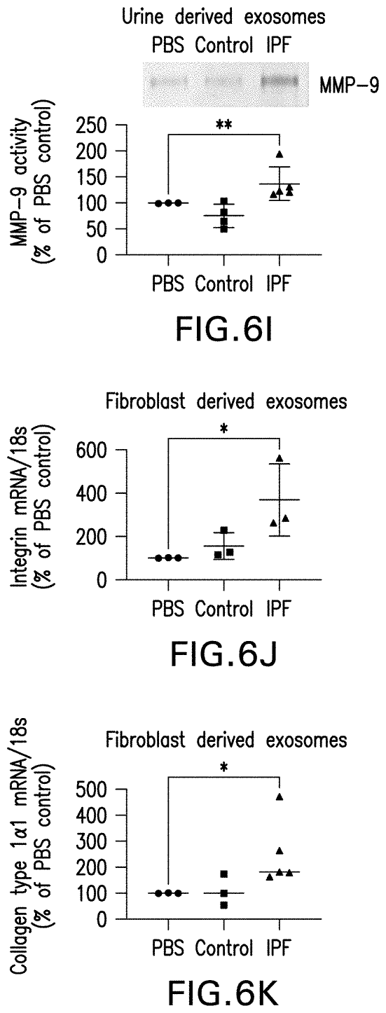

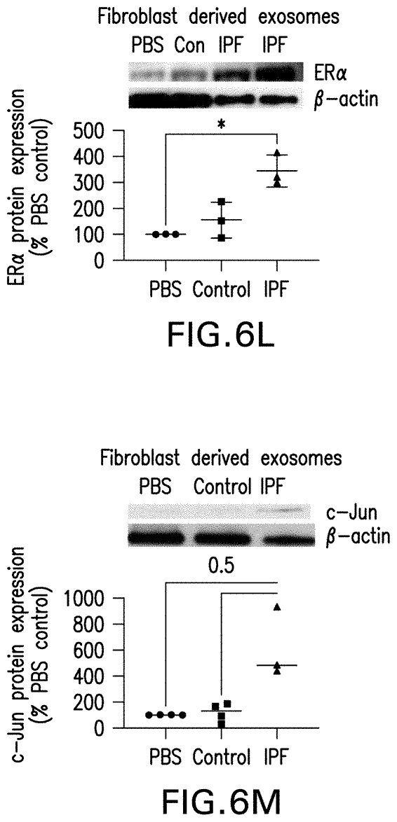

U-IPFexo stimulate a fibrotic pathway response in human lung punches ex vivo: To determine the effects of exosomes on control human lung tissue, we performed ex vivo punch experiments utilizing lungs deemed unsuitable for transplant due to trauma (n=2; FIG. 6). αᵥ-integrin and collagen type I mRNA increased after injection with MF-IPFexo compared to control (FIG. 6A). Consistent with our published data in the lungs of mice with Bleo-injury and lung tissue from individuals with IPF (27, 28), Cav-1 protein decreased (FIG. 6B) and ERα protein expression increased (FIG. 6C) after U-IPFexo were injected into human lung punches. We conducted additional experiments using lung tissue obtained from 18 month-old male C57BL/6 mice (n=3 technical replicates/individual exosome isolate). Markers for fibrosis (αv-integrin, collagen type I mRNA expression) and other downstream fibrotic pathways (c-Jun, AKT expression and MMP-9 activity) were only activated by the U-IPFexo and not control exo or PBS alone (FIG. 6D, FIG. 6E, FIG. 6F, FIG. 6G, FIG. 6H). We found a 2.5 fold increase in TGFβ mRNA expression in punches that were injected with IPF exosomes compared to those injected with PBS or control exosome injections (p<0.05). Similar data were obtained from analysis of punches injected with MF-IPFexo (FIG. 6J, FIG. 6K, FIG. 6L). mRNA expression of αv-integrin and collagen type I mRNA were increased in punches injected with MF-IPFexo compared with punches injected with age and sex-matched control fibroblast exosomes (FIG. 6J, FIG. 6K). ERα protein expression shown to be upregulated in the lung tissue and myofibroblasts isolated from individuals with IPF (27) was increased after injection of U-IPFexo (1.4 fold increase over control, p=0.05) and MF-IPFexo (>3 fold) into lung punches (FIG. 6F, FIG. 6L) compared to control exosomes. Finally, increased c-Jun expression was noted after injection of IPF exosomes but not after injection with control exosomes. We conclude that U-IPFexo stimulate a fibrotic pathway response similar to that stimulated by MF-IPFexo.

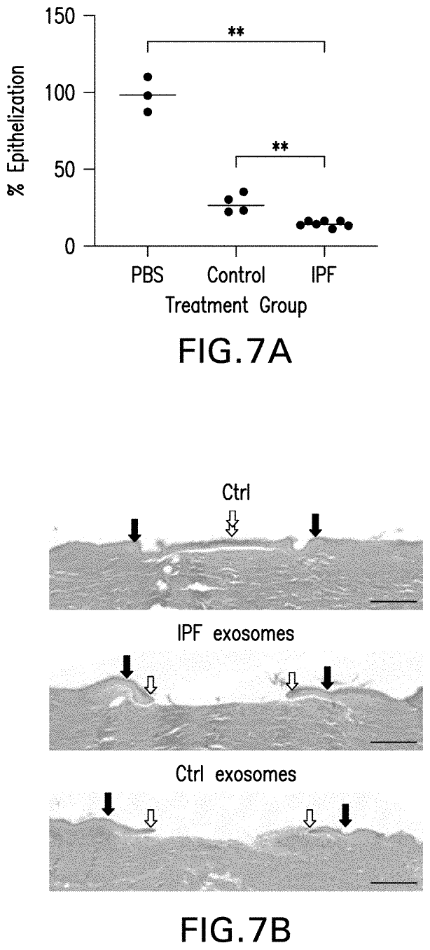

U-IPFexo impair tissue repair of human skin ex vivo: We used the human ex vivo skin wound healing assay to test whether would impair closure of wounds. U-IPFexo treatment delayed wound healing mirroring our previous data showing that mice who develop pulmonary fibrosis after treatment with Bleo also demonstrate a delay in wound healing (28). We found that U-IPFexo decreased wound closure compared to control exosomes or PBS (FIG. 7A and FIG. 7B, 27±6.3% vs. 14±0.7%, vs 95%, n=3 technical replicates from 2-3 biological exosome isolates/group), suggesting that exosomes carry a disease phenotype that can impair tissue repair. As expected, due to age of control exosomes, we noted less effective wound healing compared to the PBS control.

U-IPFexo regulate miRNA expression and MMP-9 activity: We measured miRNAs in exosome treated lung punches to determine whether expression changes found in the punches correlated with data published from lung tissue/cells isolated from individuals with IPF (25). We found decreased expression of miR-29 and miR-let-7d in punches treated with U-IPFexo (Table 4) or MF-IPFexo. MiR-199 was upregulated in those punches injected with U-IPFexo, but remained unchanged by MF-IPFexo. This was surprising since we and others have previously shown an increase in miR-199 expression in IPF lung tissue and cells and that increased miR-199 correlates with downregulated Cav-1 expression (29, 30). Additional miRNAs were assessed that were reported dysregulated in tissues, serum or sputum of individuals with IPF. Punch expression of miR-34a, and miR-142 was regulated in a manner similar to expression found in isolated exosomes after treatment with either U-IPFexo or MF-IPFexo, supporting the relevance of the cargo carried in urine of individuals with IPF (31).

TABLE 4
Table 4

| Punch miRNA | | | | | | |
|---|---|---|---|---|---|---|
| expression (% PBS) | Let-7d | miR-29 | miR-181b | miR-199 | miR-34a | miR-142 |
| Urine exosomes (n = 6-8/group) | | | | | | |
| PBS | 100 ± 0.5 | 100 ± 0.3 | 100 ± 0.2 | 100 ± 0.1 | 100 ± .3.1 | 100 ± 0.2 |
| Control exo | 74 ± 6.7 | 96 ± 13.3 | 137 ± 18 | 92 ± 5 | 88 ± 15 | 115 ± 15 |
| Urine-IPFexo | 32 ± 6.5@ | 55 ± 12.1+*& | 52 ± 17+ | 169 ± 18+& | 131 ± 10+*+ | 103 ± 18^{NS} |
| Fibroblast exosomes (n = 3-4/group) | | | | | | |
| PBS | 100 ± 0.1 | 100 ± 0.3 | 100 ± 0.5 | 100 ± 0.5 | 100 ± 0.2 | 100 ± 0.2 |
| Control exo | 114 ± 5 | 110.8 ± 9.2 | 110 ± 8.2 | 53 ± 5 | 101 ± 16.5 | 78 ± 14 |
| MF-IPFexo | 57 ± 5.9@ | 56 ± 7.9*& | 42 ± 12.7@ | 115 ± 14^{NS} | 63 ± 4.6^{NS} | 47 ± 12^{NS} |

+P = 0.05 compared to PBS,
@<0.01 compared to PBS and control exosomes,
*P < 0.05 compared to control exosomes,
&P < 0.01 compared to control exosomes,
P values were calculated by Mann-Whitney U test.
NS = not significant

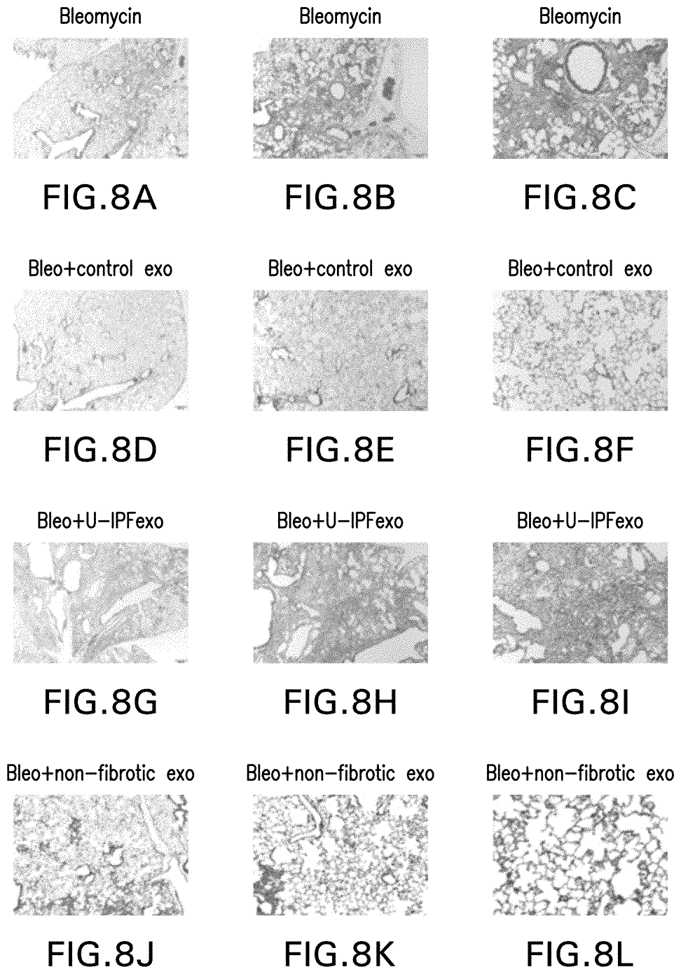

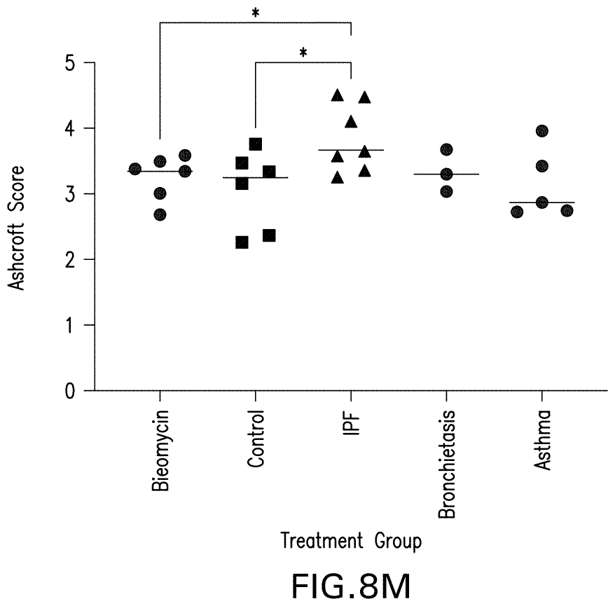

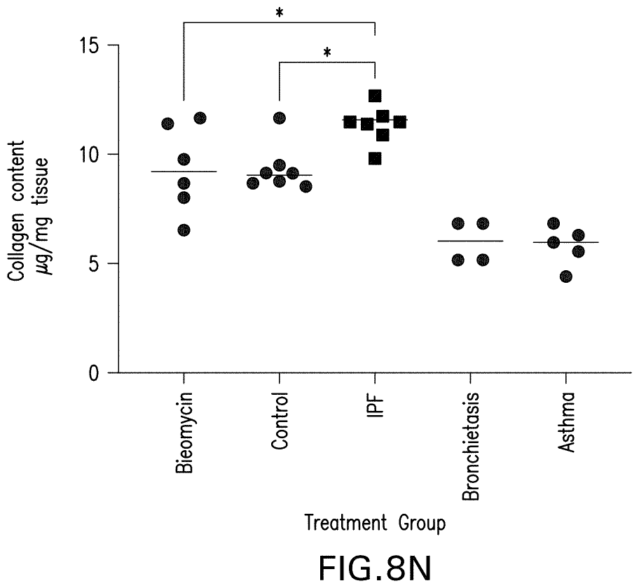

U-IPFexo impact lung fibrosis in vivo: Analysis of lung tissue obtained from Bleo treated mice sacrificed at day 21 showed a higher Ashcroft score (FIG. 8M) and collagen content (FIG. 8N) in those mice receiving U-IPFexo compared to control exosomes or Bleo alone. αV-integrin (FIG. 8O) mRNA expression was higher in lungs of mice that received U-IPFexo compared to Bleo alone. We noted higher TGFβ mRNA expression in the lung tissue of mice injected with U-IPFexo compared to Bleo treatment alone (p>0.001, 23.8±3.9 vs 11.66±0.6). Lungs obtained from mice treated with urine exosomes from either asthmatics or subjects with bronchiectasis also exhibited increased TGFβ mRNA expression. MMP-2 was induced 2.5-fold by U-IPFexo (p<0.05) compared to Bleo treatment, control exo or other exo. When Bleo injury was followed by injection of U-IPFexo, fibrosis was more homogeneous and widespread compared to Bleo treatment alone. Mice treated with IPF exosomes lost 35±2.6% BW compared to mice treated with Bleo (29±2.6% of BW; p=0.05 compared to IPF) or mice receiving control exo (19±3.2% of BW; p<0.05 compared to Bleo, p<0.01 compared to IPF). Bleo-treated Mice that received urine derived urine derived exosomes from individuals with non-CF bronchiectasis lost more weight (39±5.7% BW; p<0.05) than Bleo treated mice. The Bleo treated mice receiving urine derived exosomes from individuals with asthma lost a similar amount of weight as the Bleo treated mice (27±3.5% of BW). We also found that miR let-7d, and -142 expression decreased while miRNA miR-34a, and -199 were increased in lung tissue from Bleo mice treated with U-IPFexo (Table 5) but not urine exosomes derived from either asthmatics or subjects with bronchiectasis (Table 5). Although Ashcroft scoring and histology did not demonstrate changes consistent with fibrosis in lung tissue collected from naïve animals treated with PBS or treated

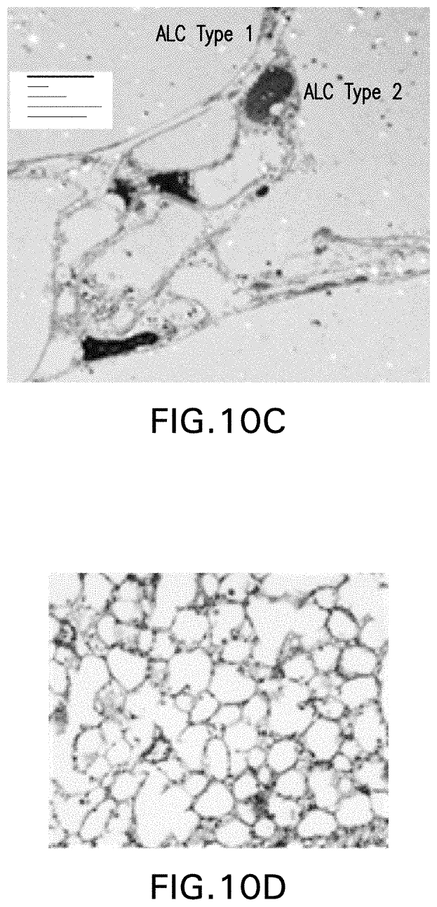

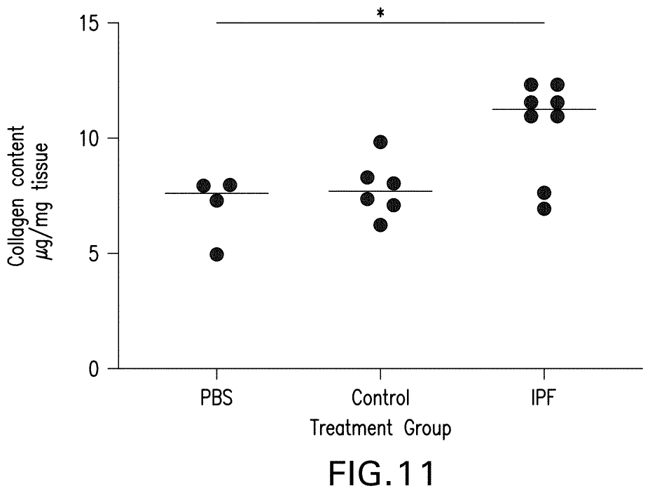

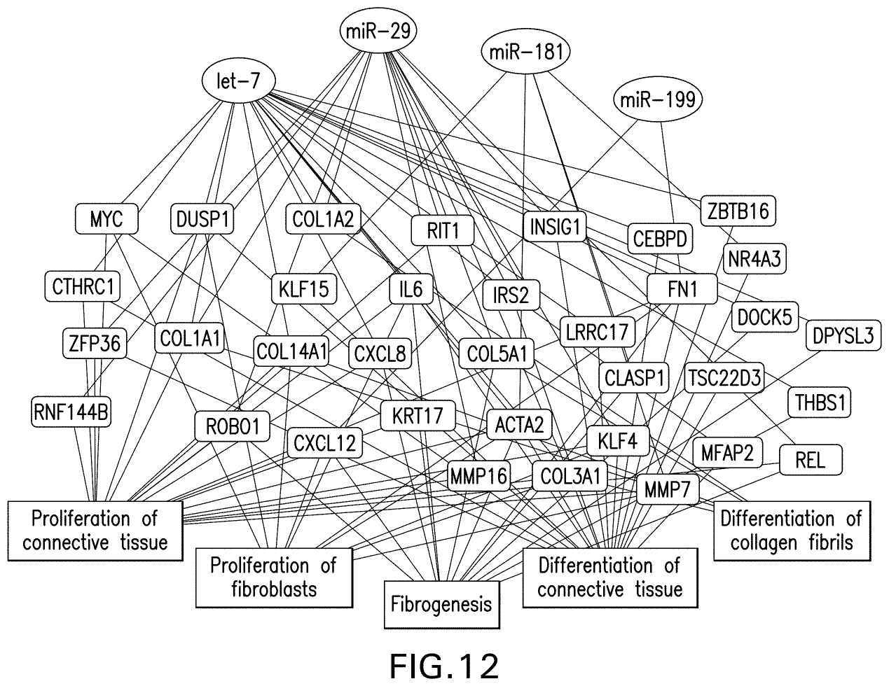

with U-IPFexo, increased collagen content (FIG. 11), dysregulated miR-34a, miR-199, miR29, and miR-142 expression (Table 5) were detected in lung tissue obtained from these mice.

Table 5 shows lung tissue microRNA expression/U6.

| | | | | | | |
|---|---|---|---|---|---|---|
| Lung Tissue | | | | | | |
| microRNA | | | | Mir-199 | Mir-34a | Mir-142 |
| expression/U6 | miR-let-7d | Mir-29 | miR-181b | fibrotic | fibrotic | antifibrotic |

| | | | | | | |
|---|---|---|---|---|---|---|
| PBS | 0.18 ± 0.03 | 2.1 ± 0.4 | ±0.26 ± 0.06 | 0.03 ± 0.001 | 0.07 ± 0.02 | 0.23 ± 0.016 |
| Control U-exo | 0.3 ± 0.01## | 2.8 ± 0.7 | 0.3 ± 0.07 | 0.06 ± 0.03 | 0.09 ± 0.01 | 0.19 ± 0.03 |
| U-IPF-exo | 0.14 ± .002 | 0.48 ± 0.1*& | .015 ± 0.05 | 0.05 ± 0.005# | 0.13 ± 0.03* | 0.16 ± 0.02* |
| Bleomycin | 0.057 ± 0.009\hat{ }\hat{ } | 0.17 ± 0.02 | 0.009 ± 0.001 | 0.19 ± 0.02 | 0.18 ± 0.05 | 0.23 ± 0.04 |
| (Bleo) | | | | | | |
| Bleo + | 0.24 ± 0.05# | 0.17 ± 0.02 | 0.008 ± 0.001 | 0.35 ± 0.11 | 0.18 ± 0.02 | 0.29 ± 0.05 |
| Control | | | | | | |
| U-exo | | | | | | |
| Bleo + | 0.10 ± 0.011*$$\hat{ }\hat{ } | 0.2 ± 0.17 | 0.004 ± 0.001# | 1.68 ± 0.44#*$$\hat{ }\hat{ } | 0.43 ± 0.08#*$$\hat{ } | 0.16 ± 0.01&$\hat{ } |
| U-IPFexo | | | | | | |
| Bleo + | 0.17 ± 0.03 | 0.14 ± 0.03 | ND | 0.13 ± 0.35 | 0.06 ± 0.02 | 0.3 ± 0.07 |
| U- | | | | | | |
| bronchiectasis | | | | | | |
| exo | | | | | | |
| Bleo + | 0.39 ± 0.1 | 0.11 ± 0.01 | ND | 0.06 ± 0.01 | 0.06 ± 0.02 | 0.29 ± 0.04 |
| U-asthma exo | | | | | | |

*P < 0.05 compared to control exo,
&P < 0.001 compared to control exosomes;
#P < 0.05 compared to Bleo,
##P < 0.001 compared to Bleo,
$P < 0.05,
$$P < 0.01 compared to bronchiectasis exo
\hat{ }P < 0.01
\hat{ }\hat{ }P < 0.001 compared to asthma exo.
P values were calculated by one way analysis of variation (ANOVA) and Mann-Whitney U test.
ND = not detected.

Discussion

EVs isolated from various tissues, cells and body fluids contain bioactive molecules such as proteins, lipids, and RNAs derived from the cell of origin (32), (33). Most recently, studies on exosomes have focused on miRNAs, small noncoding RNA molecules able to influence protein expression. Since exosomes act as an interface for cell-cell communication, we investigated the ability of “diseased” exosomes to promote fibrotic lung disease and impair wound healing. Our study is the first to report that U-IPFexo confer a disease phenotype in vivo and in ex vivo lung punches and impair ex vivo wound healing in mouse and human tissue. In a similar manner, MF-IPFexo activated markers of fibrosis in ex vivo lung punches compared to punches treated with lung fibroblast-derived exosomes isolated from the lung tissue of age and sex-matched individuals without fibrotic lung disease.

Changes in miRNAs have been implicated in gene expression associated with the development of IPF (15, 16, 18). We found dysregulated expression of miRNAs; let-7, miR-29a, miR-181b and miR-199 in the isolated U-IPFexo compared to control exosomes. These miRNAs are known to regulate expression of pro-fibrotic, inflammatory and ECM encoding genes (16, 19, 34-37) and found in lung, serum and sputum of individuals with IPF (15, 18, 19, 27, 38, 39).

Micro RNA Let-7 regulates ERα protein expression as well as the regulation of downstream fibrotic inducers including TGF-β cell signaling pathways (27). Dysregulation of miRNA let-7 contributes to endothelial/epithelial to mesenchymal transition (Endo/EMT) in the lung (18, 19), heart (37) and kidney (40), often leading to fibrosis. Njock et al. reported a positive correlation between diffusing capacity of the lungs for carbon monoxide/alveolar volume (DLCO/VA) and presence of let-7d (16). Dysregulation of miR-29 in the lung alters expression of MMP-2 and collagen1α1, while miR-199 regulates caveolin-1 (18, 28, 41-43). Rescue of miR-181b, an inhibitor of NF-κB targets, suppressed TGFβ in burns and in a Bleo model of IPF (44, 45). Increasing miR-181b expression mitigated TGF-β1 induced EMT in vitro and alleviated alveolar septal thickening and decreased collagen and MMP expression in vivo (45).

Collectively these data suggest the exosomes can deliver expression of dysregulated miRNAs and may promote a disease phenotype. When distinct regions were histologically analyzed from the same IPF lung and the histology labeled as minimal, moderate and end-stage disease severity divergent sets of genes and miRNA expression were dependent on the severity of diseased tissue (38). MicroRNA let-7, a critical marker in our studies, was associated with end-stage fibrotic lung disease. In a recent study, PBMC miRNA expression correlated with survival in individuals with IPF (24), supporting the disease conferring role of miRNA expression as a potential signature of disease. In the present study, we compared expression of miRNAs from urine-derived exosomes and serum-derived exosomes isolated from the same individuals with IPF. We acknowledge that the absence of differences in miRNA expression between urine and serum-derived exosomes may reflect our small sample size. These data warrant a larger study, that is currently ongoing.

To demonstrate delivery of exosomes to the lungs, we performed biodistribution experiments using U-IPFexo and urine exosomes from control subjects (without lung disease). The urine-derived exosomes were located in the lungs of 18-month old male mice within 5 minutes followed by a diminished signal at 24-48 hours; similar transit time was recorded whether the urine exosomes were prepared from individuals with IPF or controls. Additional studies showed that an exosome signal was no longer visualized in the mouse at 19 days post-injection. Our studies uses ExoGlow™-Vivo that employs an amine binding dye that emits in the near infrared (NIR) range instead of infrared lipophilic cyanine dye, DiIC18 (DiD) to label urine derived exosomes (5). Since the ExoGlow dye is non-lipophilic, it provides a more specific signal when used in vivo. Similar transit times have been reported using DiD to label bone marrow mesenchymal stem cell-derived exosomes showing delivery after intravenous infusion to the lung at 5 minutes and to the liver and spleen at 24 hours (46). Exosomes derived from breast cancer cells have a similar profile of biodistribution: lung, >liver>spleen, kidney>heart>bone marrow (47).

To study cellular and tissue interplay in the lung (48, 49) we evaluated how the exosomes integrated in ex vivo lung punches by loading exosomes with nanoparticles and performing TEM (50, 51). We found that the punch architecture was preserved after injection of exosomes and that the urine-derived exosomes were integrated into AEC I or AEC II cells. Our studies do not distinguish whether homing of the “diseased” exosomes favored a specific cell type compared to injection with control exosomes. These extensive studies are ongoing in our laboratory.

Further studies using ex vivo lung punches from human non-fibrotic lung tissue and naïve 18-month old male mice noted minimal effect on the histology of the lung punch after treatment with PBS or urine-derived exosomes isolated from age and sex-matched controls without lung disease. We found that treatment with U-IPFexo increased expression of fibrotic markers and pathways (αv-integrin, collagen type I and TGFβ mRNA expression, c-Jun protein expression and MMP-9 and AKT activation). Additionally, there was a notable decrease in SPC positive cells in punches (AEC II cells) receiving U- or MF-IPFexo. Since AEC II are regarded as the progenitor population of the alveolus responsible for injury repair and homeostatic maintenance, these data suggest that IPFexo may be inhibiting the repair mechanism.

We have previously reported an increase of ERα protein expression in cells and tissue isolated from lungs of male individuals with IPF (27). Studies suggest that activation of ERα may be profibrotic in multiple organs including the lung (52, 53). In the current investigation we found at least ˜1.4-fold increase in ERα protein expression in human and mouse punches treated with U-IPFexo or MF-IPFexo. Punches injected with MF-IPFexo also induced other components of fibrotic pathways consistent with data derived from punches injected with U-IPFexo. Furthermore, U-IPFexo impaired the wound healing response, supporting our hypothesis that exosome cargo delivered signals impairing wound repair in skin. This suggests that the systemic feature of IPF exosomes may predispose impaired tissue healing.

We recognize that under our experimental conditions, lung punches lack immune recruitment, systemic perfusion and are under potentially hypoxic conditions. Therefore, we sought to determine if these exosomes would confer lung injury in vivo under normoxic conditions in old male C57BL/6 mice. We injected U-IPFexo into naive 18-month-old C57BL/6 mice and collected the lungs 21 days post-exosome treatment. Although there was no change in Ashcroft score, we noted increased collagen content (measured by hydroxyproline) and evidence of inflammatory changes, including increased macrophages in lung tissue from mice treated with U-IPFexo compared to tissue from mice treated with control exosomes. Another group of mice were treated with Bleo. Ashcroft score, collagen content, αV-integrin and TGFβ mRNA expression increased in the lungs of mice treated with Bleo+U-IPFexo compared to mice treated with Bleo+vehicle and Bleo+control exosomes, suggesting that U-IPFexo escalated fibrotic pathways induced by Bleo in old male mice. There was no increase of TGFβ in lungs of mice treated with non-fibrotic exo. As reviewed, TGFB is ubiquitous in chronic inflammatory lung diseases including COPD, IPF and asthma and may contribute to an immune-suppressed state (Thomas, B J et al. Am. J. Respir. Cell Mol. Biol. (2016) 55(6): 759-66).

We also found an expected increase in MMP-2 activity in the lungs of U-IPFexo treated mice, as MMP-2 increases migration and invasiveness of lung myofibroblasts during Bleo-induced pulmonary fibrosis (54). This was in contrast to urine derived control-exosomes or exosomes from urine of individuals with bronchiectasis or asthma. BW, historically shown to decrease with Bleo treatment (55) was decreased in mice receiving Bleo+U-IPFexo compared to Bleo alone or Bleo+control exosomes. Loss of BW is associated with worse survival in individuals with IPF (21, 22). Our studies also found similar changes in miRNA expression as reported in prior studies from patients with IPF (14-18, 25-27, 31, 41, 56-58).

Our study demonstrates that U-IPFexo preparations contain disease invoking cargo. Two recent studies by Martin-Medina et al (59) and Parimon et al. (17) showed that antifibrotic microRNAs (miR 144-3p, miR144-3p, miR 34-5p, miR 503-5) packaged into EVs regulate epithelial plasticity and potential pulmonary fibrosis and reported an increase in the number of EV in BALF (bronchoalveolar lavage fluid) from Bleo-treated mice as well as from patients with IPF that function as carriers for signaling mediators, such as WNT5A. Both studies reported that isolated EV preparations consisted predominantly of exosomes, although microvesicles were present, unlike our preparations that did not contain microvesicles. Neither study investigated urine-derived exosomes. In the described study, lung tissue isolated from naïve or Bleo-treated mice injected with U-IPFexo also exhibited the same changes in miRNA expression as found in ex vivo lung punches. The present study illustrates the correlation between changes in mRNA and protein expression (TGFβ, MMPs) and dysregulated miRNA expression suggesting that a miRNA signature could function as a potential biomarker for fibrotic lung disease. Reproducible biomarkers would enable early diagnosis and non-invasive detection methods are currently under investigation for multiple diseases (60).

We further analyzed lungs from Bleo mice that were treated with exosomes derived from subjects with asthma or bronchiectasis, non-fibrotic lung diseases. We did not find an increase in the fibrotic markers studied and in fact noted a decrease in collagen content and integrin in those lungs analyzed. We recognize that this small set of mice will need to be expanded in future experiments and also noted that the subjects with asthma were not age matched to the control or IPF subjects. However, the decreased expression of miR-34a and miR-199, fibrotic inducing microRNA highlight the differences in cargo content between exosomes from various lung diseases (61, 62).

In an effort to detect expression of other potential fibrotic pathways, we analyzed the urine-exosomes for mRNA expression of TGFβ, IGF, and Cav-1 (63-65). Because exosomes contain non-coding and other small coding RNAs, derived from transfer RNA (tRNA), ribosomal RNA (rRNA), small nuclear RNA (snRNA) and small nucleolar RNA (snoRNA) (66-69), we attempted to characterize the more easily studied expression of mRNA and mRNA fragments (70, 71). As expected, we found no evidence of mRNA expression for our selected targets (supplement). However, the presence of 3′ UTR mRNA fragments which could bind miRNA and allow for RNA transcription to proceed in target cells ultimately altering the phenotype of otherwise healthy cells has been reported (71).

Our study provides the first evidence of circulating cargo found in U-IPFexo that confers disease in two target organs (lungs and skin) and suggests a potential mechanism for initiation and/or progression of disease. Urine-derived exosomes potentially represent a novel way to identify biomarkers for lung and fibrotic diseases. Our data further suggest that increased miRNA profiling of urine-derived exosomes as biomarkers may lead to noninvasive assessments for earlier diagnosis. Overlap of miRNA expression yielding a fibrotic profile to produce a personalized panel ofurine-derived miRNAs or fibro miRs to detect IPF and other lung and fibrotic diseases may be a diagnostic and prognostic tool.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

### REFERENCES

1. J. F. Cordier, V. Coffin, Neglected evidence in idiopathic pulmonary fibrosis: from history to earlier diagnosis. Eur Respir. 1, 42, 916-923 (2013).
2. D. J. Lederer, F. J. Martinez, Idiopathic Pulmonary Fibrosis. NEngl. 1, Med 379, 797-798 (2018).
3. L. Richeldi, H. R. Collard, M. G. Jones, Idiopathic pulmonary fibrosis. Lancet 389, 1941-1952 (2017).
4. F. J. Martinez et al., Idiopathic pulmonary fibrosis. Nat Rev Dis Primers 3, 17074 (2017).
5. G. Raghu, S. Y. Chen, Q. Hou, W. S. Yeh, H. R. Collard, Incidence and prevalence of idiopathic pulmonary fibrosis in US adults 18-64 years old. Eur Respir. 1, 48, 179-186 (2016).
6. L. Hecker, Mechanisms and consequences of oxidative stress in lung disease: therapeutic implications for an aging populace. Am 0.1. Physiol Lung Cell Mol Physiol 314, L642-L653 (2018).
7. T. Kinoshita, T. Goto, Molecular Mechanisms of Pulmonary Fibrogenesis and Its Progression to Lung Cancer: A Review. International journal of molecular sciences 20, 1461 (2019).
8. A. Tzouvelekis, H.-M. Jose, S. Koji, D. Bourns, Biomarkers in the Evaluation and Management of Idiopathic Pulmonary Fibrosis. Current Topics in Medicinal Chemistry 16, 1587-1598 (2016).
9. F. Drakopanagiotakis, L. Wujak, M. Wygrecka, P. Markart, Biomarkers in idiopathic pulmonary fibrosis. Matrix Biology 68-69, 404-421 (2018).
10. M. H Rashed et al., Exosomes: From Garbage Bins to Promising Therapeutic Targets. International journal of molecular sciences 18, 538 (2017).
11. M. Yaliez-Mó et al., Biological properties of extracellular vesicles and their physiological functions. Journal of extracellular vesicles 4, 27066-27066 (2015).
12. S. Keller, J. Ridinger, A.-K. Rupp, J. W. G. Janssen, P. Altevogt, Body fluid derived exosomes as a novel template for clinical diagnostics. Journal of translational medicine 9, 86-86 (2011).
13. G. Raposo, W. Stoorvogel, Extracellular vesicles: exosomes, microvesicles, and friends. J Cell Biol 200, 373-383 (2013).
14. J. Guiot et al., Exosomal miRNAs in Lung Diseases: From Biologic Function to Therapeutic Targets. J Clin Med 8, (2019).
15. G. Yang et al., Discovery and validation of extracellular/circulating microRNAs during idiopathic pulmonary fibrosis disease progression. Gene 562, 138-144 (2015).
16. M.-S. Njock et al., Sputum exosomes: promising biomarkers for idiopathic pulmonary fibrosis. Thorax 74, 309 (2019).
17. T. Parimon et al., Syndecan-1 promotes lung fibrosis by regulating epithelial reprogramming through extracellular vesicles. JCI Insight 5, (2019).
18. K. V. Pandit, J. Milosevic, MicroRNA regulatory networks in idiopathic pulmonary fibrosis. Biochem Cell Biol 93, 129-137 (2015).
19. S. Rajasekaran, P. Rajaguru, P. S. Sudhakar Gandhi, MicroRNAs as potential targets for progressive pulmonary fibrosis. Front Pharmacol 6, 254 (2015).
20. A. C. Habermann et al., Single-cell RNA-sequencing reveals profibrotic roles of distinct epithelial and mesenchymal lineages in pulmonary fibrosis. bioRxiv, 753806 (2019).
21. Y. Nakatsuka et al., The Clinical Significance of Body Weight Loss in Idiopathic Pulmonary Fibrosis Patients. Respiration 96, 338-347 (2018).
22. N. Mogulkoc et al., Does body mass index have prognostic significance for patients with idiopathic pulmonary fibrosis? European Respiratory Journal 52, PA2210 (2018).
23. S. Gurunathan, M.-H. Kang, M. Jeyaraj, M. Qasim, J.-H. Kim, Review of the Isolation, Characterization, Biological Function, and Multifarious Therapeutic Approaches of Exosomes. Cells 8, 307 (2019).
24. N. G. Casanova et al., MicroRNA and protein-coding gene expression analysis in idiopathic pulmonary fibrosis yields novel biomarker signatures associated to survival. Translational Research 228, 1-12 (2021).
25. N. Omote, M. Sauler, Non-coding RNAs as Regulators of Cellular Senescence in Idiopathic Pulmonary Fibrosis and Chronic Obstructive Pulmonary Disease. Frontiers in Medicine 7, (2020).
26. B. Ortiz-Quintero et al., Circulating microRNA Signature Associated to Interstitial Lung Abnormalities in Respiratory Asymptomatic Subjects. Cells 9, (2020).
27. S. Elliot et al., MicroRNA let-7 Downregulates Ligand-Independent Estrogen Receptor-mediated Male-Predominant Pulmonary Fibrosis. Am JRespir Crit Care Med 200, 1246-1257 (2019).
28. G. A. Rubio et al., Mesenchymal stromal cells prevent Bleomycin-induced lung and skin fibrosis in aged mice and restore wound healing. J Cell Physiol 233, 5503-5512 (2018).
29. J. F. Aranda, A. Canfran-Duque, L. Goedeke, Y. Suarez, C. Fernandez-Hernando, The miR-199-dynamin regulatory axis controls receptor-mediated endocytosis. J Cell Sci 128, 3197-3209 (2015).
30. G. A. Rubio, P. Catanuto, M. K. Glassberg, J. I. Lew, S. J. Elliot, Estrogen receptor subtype expression and regulation is altered in papillary thyroid cancer after menopause. Surgery 163, 143-149 (2018).
31. S. K. Shetty et al., p53 and miR-34a Feedback Promotes Lung Epithelial Injury and Pulmonary Fibrosis. Am J Pathol 187, 1016-1034 (2017).
32. F. Collino et al., Microvesicles derived from adult human bone marrow and tissue specific mesenchymal stem cells shuttle selected pattern of miRNAs. PLoS One 5, e 1 1 803 (2010).
33. F. J. Vizoso, N. Eiro, S. Cid, J. Schneider, R. Perez-Fernandez, Mesenchymal Stem Cell Secretome: Toward Cell-Free Therapeutic Strategies in Regenerative Medicine. Int JMol Sci 18, (2017).
34. J. E. McDonough et al., Gene correlation network analysis to identify regulatory factors in idiopathic pulmonary fibrosis. Thorax 74, 132-140 (2019).
35. P. Minnis et al., Serum exosomes from IPF patients display a fibrotic miRNA profile that correlates to clinical measures of disease severity. European Respiratory Journal 46, PA3845 (2015).
36. A. Noetel, M. Kwiecinski, N. Elfimova, J. Huang, M. Odenthal, microRNA are Central Players in Anti- and Profibrotic Gene Regulation during Liver Fibrosis. Front Physiol 3, 49 (2012).
37. J. Wang, O. W. Liew, A. M. Richards, Y.-T. Chen, Overview of MicroRNAs in Cardiac Hypertrophy, Fibrosis, and Apoptosis. International journal of molecular sciences 17, 749 (2016).
38. J. E. McDonough et al., Transcriptional regulatory model of fibrosis progression in the human lung. JCI Insight 4, (2019).
39. K. V. Pandit et al., Inhibition and Role of let-7d in Idiopathic Pulmonary Fibrosis. American Journal of Respiratory and Critical Care Medicine 182, 220-229 (2010).
40. S. P. Srivastava, J. E. Goodwin, K. Kanasaki, D. Koya, Inhibition of Angiotensin-Converting Enzyme Ameliorates Renal Fibrosis by Mitigating DPP-4 Level and Restoring Antifibrotic MicroRNAs. Genes (Basel) 11, (2020).
41. L. Cushing et al., miR-29 is a major regulator of genes associated with pulmonary fibrosis. Am JRespir Cell Mol Biol 45, 287-294 (2011).
42. R. L. Montgomery et al., MicroRNA mimicry blocks pulmonary fibrosis. EMBO Mol Med 6, 1347-1356 (2014).
43. T. Yang et al., miR-29 mediates TGFbetal-induced extracellular matrix synthesis through activation of PI3K-AKT pathway in human lung fibroblasts. J Cell Biochem 114, 1336-1342 (2013).
44. X. Sun et al., MicroRNA-18 lb regulates NF-kappaB-mediated vascular inflammation. J Clin Invest 122, 1973-1990 (2012).
45. L. Song, D. Li, Toll like receptor 4 activation upregulates miR-18 lb attenuating pulmonary fibrosis via targeting TGFBR1. European Respiratory Journal 46, OA2933 (2015).
46. C. Grange et al., Biodistribution of mesenchymal stem cell-derived extracellular vesicles in a model of acute kidney injury monitored by optical imaging. Int JMol Med 33, 1055-1063 (2014).
47. S. W. Wen et al., The Biodistribution and Immune Suppressive Effects of Breast Cancer-Derived Exosomes. Cancer Research 76, 6816 (2016).
48. F. E. Uhl et al., Preclinical validation and imaging of Wnt-induced repair in human 3D lung tissue cultures. Eur Respir J 46, 1150-1166 (2015).
49. K. Zscheppang et al., Human Pulmonary 3D Models For Translational Research.

Biotechnol J13, (2018).
50. O. Betzer et al., In Vivo Neuroimaging of Exosomes Using Gold Nanoparticles. ACS Nano 11, 10883-10893 (2017).
51. E. Zeringer et al., Methods for the extraction and RNA profiling of exosomes. World J Methodol 3, 11-18 (2013).
52. A. Pedram, M. Razandi, F. O'Mahony, D. Lubahn, E. R. Levin, Estrogen receptor-beta prevents cardiac fibrosis. Mol Endocrinol Baltim Md. 24, (2010).
53. H. Zhao et al., Shift from androgen to estrogen action causes abdominal muscle fibrosis, atrophy, and inguinal hernia in a transgenic male mouse model. Proceedings of the National Academy of Sciences 115, E10427-E10436 (2018).
54. B. Singh, R. K. Kasam, V. Sontake, T. A. Wynn, S. K. Madala, Repetitive intradermal Bleomycin injections evoke T-helper cell 2 cytokine-driven pulmonary fibrosis. Am 0.1. Physiol Lung Cell Mol Physiol 313, L796-L806 (2017).
55. P. M. Cowley, C. R. Roberts, A. J. Baker, Monitoring the Health Status of Mice with Bleomycin-induced Lung Injury by Using Body Condition Scoring. Comp Med 69, 95-102 (2019).
56. S. Disayabutr et al., miR-34 miRNAs Regulate Cellular Senescence in Type II Alveolar Epithelial Cells of Patients with Idiopathic Pulmonary Fibrosis. PLoS One 11, e0158367 (2016).
57. T. Argyris, H.-M. Jose, S. Koji, B. Demosthenes, Biomarkers in the Evaluation and Management of Idiopathic Pulmonary Fibrosis. Current Topics in Medicinal Chemistry 16, 1587-1598 (2016).
58. C. L. Lino Cardenas, N. Kaminski, D. J. Kass, Micromanaging microRNAs: using murine models to study microRNAs in lung fibrosis. Drug Discovery Today: Disease Models 10, e145-el51 (2013).
59. A. Martin-Medina et al., Increased Extracellular Vesicles Mediate WNTSA Signaling in Idiopathic Pulmonary Fibrosis. Am JRespir Crit Care Med 198, 1527-1538 (2018).
60. A. L. Tatler, Recent advances in the non-invasive assessment of fibrosis using biomarkers. Curr Opin Pharmacol 49, 110-115 (2019).
61. J. Weidner et al., Spotlight on microRNAs in allergy and asthma. Allergy 76, 1661-1678 (2021).
62. K. Usman, A. Hsieh, T. L. Hackett, The Role of miRNAs in Extracellular Matrix Repair and Chronic Fibrotic Lung Diseases. Cells 10, (2021).
63. N. G. Frangogiannis, Transforming growth factor-0 in tissue fibrosis. Journal of Experimental Medicine 217, (2020).
64. S. M. Garrett, E. Hsu, J. M. Thomas, J. M. Pilewski, C. Feghali-Bostwick, Insulin-like growth factor (IGF)-II-mediated fibrosis in pathogenic lung conditions. PLoS One 14, e0225422 (2019).
65. S. A. Wicher, Y. S. Prakash, C. M. Pabelick, Caveolae, caveolin-1 and lung diseases of aging. Expert Rev Respir Med 13, 291-300 (2019).
66. J. Perez-Boza, M. Lion, I. Struman, Exploring the RNA landscape of endothelial exosomes. RNA (New York, N.Y.) 24, 423-435 (2018).
67. J. Lv et al., Identification and characterization of long intergenic non-coding RNAs related to mouse liver development. Mol Genet Genomics 289, 1225-1235 (2014).
68. L. Qu et al., Exosome-Transmitted lncARSR Promotes Sunitinib Resistance in Renal Cancer by Acting as a Competing Endogenous RNA. Cancer Cell 29, 653-668 (2016).
69. C. Berrondo et al., Expression of the Long Non-Coding RNA HOTAIR Correlates with Disease Progression in Bladder Cancer and Is Contained in Bladder Cancer Patient Urinary Exosomes. PLoS One 11, e0147236 (2016).
70. P. W. Peake et al., A comparison of the ability of levels of urinary biomarker proteins and exosomal mRNA to predict outcomes after renal transplantation. PLoS One 9, e98644 (2014).
71. A. O. Batagov, I. V. Kurochkin, Exosomes secreted by human cells transport largely mRNA fragments that are enriched in the 3′-untranslated regions. Biology direct 8, 12-12 (2013).
72. J. Tashiro et al., Therapeutic benefits of young, but not old, adipose-derived mesenchymal stem cells in a chronic mouse model of Bleomycin-induced pulmonary fibrosis. Transl Res 166, 554-567 (2015).
73. M. Karl, M. Berho, J. Pignac-Kobinger, G. E. Striker, S. J. Elliot, Differential effects of continuous and intermittent 17beta-estradiol replacement and tamoxifen therapy on the prevention of glomerulosclerosis: modulation of the mesangial cell phenotype in vivo. The American journal of pathology 169, 351-361 (2006).
74. M. Potier et al., Estrogen-related abnormalities in glomerulosclerosis-prone mice: reduced mesangial cell estrogen receptor expression and prosclerotic response to estrogens. The American journal of pathology 160, 1877-1885 (2002).
75. T. D. Schmittgen, K. J. Livak, Analyzing real-time PCR data by the comparative C(T) method. Nat Protoc 3, 1101-1108 (2008).
76. M. K. Glassberg et al., 1 7beta-estradiol replacement reverses age-related lung disease in estrogen-deficient C57BL/6J mice. Endocrinology 155, 441-448 (2014).
77. T. Ashcroft, J. M. Simpson, V. Timbrell, Simple method of estimating severity of pulmonary fibrosis on a numerical scale. Journal of clinical pathology 41, 467-470 (1988).

What is claimed is:

1. A method for noninvasively diagnosing and staging a progressive chronic lung disease characterized by disease related lung dysfunction comprising:
(a) quantifying lung function of a subject with a progressive chronic lung disease characterized by disease-related lung dysfunction;
(b) diagnosing and determining a stage of the chronic lung disease by:
(i) collecting a biological sample from the subject with the lung disease and from a healthy control,
(ii) centrifuging the biological sample at low speed to form a clarified supernatant;
(iii) ultracentrifuging the clarified supernatant to pellet the purified enriched population of exosomes; and
(iv) characterizing the purified enriched population of exosomes, wherein.
(1) the population of exosomes expresses two or more exosome biomarkers selected from the group consisting of CD9, CD63, CD81, or HSP70;
(2) size of the exosomes in the population of exosomes ranges from 30 m to 150 μm, inclusive;
(3) the population of exosomes comprises a total protein of at least 100-200 μg, inclusive;
(4) the population of exosomes comprise a total RNA content of at least 100-200 ng, inclusive;
(5) number of exosomes in the population of exosomes comprises at least 10E10 particles; and
(6) the population of exosomes comprises a cargo comprising dysregulated expression of two or more microRNAs selected from miR-199, miR Let-7a, miR Let-7b, mir-Let-7d, miR-10a, miR-21, miR-29a, miR-34, miR-101, miR-125, miR-145, miR-146a, miR-181a, miR-181b, miR-181c, miR-199, and miR-142,
wherein expression of the two or more microRNAs, compared to a healthy control, comprises a signature of a fibrotic lung disease; and
(c) medically managing the diagnosed fibrotic lung disease as early as possible in the course of progression of the disease to reduce or slow its progression by
(1) treating the diagnosed fibrotic lung disease; and
(2) monitoring over time the population of exosomes derived from the biological sample of the subject compared to a population of exosomes derived from a biological sample derived from a healthy control.

2. The method according to claim 1, wherein the lung function of the subject is quantified by determining forced expiratory volume (FEV1); forced vital capacity (FVC) and FEV/FVC %.

3. The method according to claim 1, wherein the biological sample is a body fluid and the body fluid is amniotic fluid, blood, breast milk, saliva, urine, bile, pancreatic juice, cerebrospinal fluid or a peritoneal fluid.

4. The method according to claim 3, wherein the body fluid is serum or urine

5. The method according to claim 4, wherein the body fluid is serum.

6. The method according to claim 4, wherein the body fluid is urine.

7. The method according to claim 1, wherein the biological sample is obtained from a mammal.

8. The method according to claim 1, wherein the biological sample is obtained from a non-human mammal or a human subject.

9. The method according to claim 1, wherein the healthy control is age and sex matched to the subject.

10. The method according to claim 1, wherein the chronic lung disease if left untreated comprises one or more of a progressive injury, progressive inflammation, progressive fibrosis or a combination thereof.

11. The method according to claim 10, wherein the fibrotic lung disease is interstitial pulmonary fibrosis (IPF).

12. The method according to claim 11,
(a) wherein dysregulated miRNA expression of miR-199 and miR-34a in the population of exosomes derived from the biological sample of the subject compared to a population of exosomes derived from a biological sample derived from a healthy control is fibrotic; or
(b) wherein dysregulated miRNA expression of miR-142 in the population of exosomes derived from the biological sample of the subject compared to a population of exosomes derived from a biological sample derived from a healthy control is antifibrotic; or
(c) wherein the dysregulated expression of one or more of miR let-7D, miR-29, or miR-181 in the population of exosomes derived from the biological sample of the subject compared to a population of exosomes derived from a biological sample derived from a healthy control is decreased, compared to the population of exosomes derived from the healthy control; or
(d) wherein dysregulated expression of miR-199 in the population of exosomes derived from the biological sample of the subject compared to a population of exosomes derived from a biological sample derived from a healthy control is increased and the expression of one or more of miR-let-7d, miR-29, miR-142, miR-181 in the population of exosomes derived from the biological sample of the subject compared to a population of exosomes derived from a biological sample derived from a healthy control is decreased, or
(e) wherein reduced or absent miR Let-7 expression in the population of exosomes derived from the biological sample of the subject compared to a population of exosomes derived from a biological sample derived from a healthy control compared to a healthy control is associated with early stage fibrotic lung disease; or
(f) wherein micro RNA expression in the population of exosomes derived from the biological sample of the subject compared to a population of exosomes derived from a biological sample derived from a healthy control comprises increased miR-Let-7 expression compared to a healthy control and is associated with end-stage fibrotic lung disease.

13. The method according to claim 1, wherein the medical managing modulates one or more of an injury, inflammation, an excess accumulation of extracellular matrix, cell senescence; or a pathway comprising fibrogenic signaling.

14. The method according to claim 13, wherein markers for fibrosis and downstream fibrotic pathways comprise αᵥ-integrin, collagen type I mRNA expression, c-Jun, AKT expression and MMP-9 activity.

15. The method according to claim 13, wherein the pathway comprising fibrogenic signaling is one or more of a Smad pathway, a mitogen-activated protein kinase pathway, a phosphoinositide 3-kinase pathway; a canonical Wnt-β catenin pathway, and a Notch signaling pathway.

16. The method according to claim 13, wherein the pathway comprising fibrogenic signaling comprises transforming growth factor (TGFβ) signaling.

17. The method according to claim 1, wherein the treating of the fibrotic disease includes administering an active therapeutic agent including an immunomodulator, an analgesic, an anti-inflammatory agent, an anti-fibrotic agent, an anti-viral agent, a proton pump inhibitor, or oxygen therapy.

18. The method according to claim 17, wherein the immunomodulator includes prednisone, azathioprine, mycophenolate, mycophenolate mofetil, colchicine, or interferon-gamma 1b.

19. The method according to claim 17, wherein the analgesic includes capsaisin, codeine, hydrocodone, lidocaine, oxycodone, methadone, resiniferatoxin, hydromorphone, morphine, and fentanyl.

20. The method according to claim 17, wherein the anti-inflammatory agent includes aspirin, celecoxib, diclofenac, diflunisal, etodolac, ibuprofen, indomethacin, ketoprofen, ketorolac nabumetone, naproxen, nintedanib, oxaprozin, pirfenidone, piroxicam, salsalate, sulindac, and tolmetin.

21. The method according to claim 17, wherein the anti-fibrotic agent includes nintedanib or pirfenidone.

22. The method according to claim 17, wherein the anti-viral agent includes acyclovir, gancidovir, foscarnet; ribavirin; amantadine, azidodeoxythymidine/zidovudine), nevirapine, a tetrahydroimidazobenzodiazepinone (TIBO) compound; efavirenz; remdecivir, or delavirdine.

23. The method according to claim 17, wherein the proton pump inhibitor includes omeprazole, lansoprazole, dexlansoprazole, esomeprazole, pantoprazole, rabeprazole, or ilaprazole.
